SEQUENCE LISTING (73 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 223 aa, Severe acute respiratory syndrome coronavirus 2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 3 - protein, 32 aa, synthetic construct
GSGYIPEAPRDGQAYVRKDGEWVLLSTFLGRS

SEQ ID NO: 4 - protein, 33 aa, synthetic construct
RMKQIEDKIEEILSKIYHIENEIARIKKLIGER

SEQ ID NO: 5 - protein, 10 aa, synthetic construct
GGGGSGGGGS

SEQ ID NO: 6 - protein, 588 aa, Severe acute respiratory syndrome coronavirus 2
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 7 - protein, 588 aa, Severe acute respiratory syndrome coronavirus 2
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 8 - protein, 1272 aa, Severe acute respiratory syndrome coronavirus 2
FVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 9 - protein, 1268 aa, Severe acute respiratory syndrome coronavirus 2
FVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSRSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 10 - protein, 1268 aa, Severe acute respiratory syndrome coronavirus 2
FVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSRSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 11 - DNA, 1764 bp, Severe acute respiratory syndrome coronavirus 2
  source 1..1764: mol_type = unassigned DNA
agtgtggcttctcaaagcattatagcatacactatgtctcttggtgccgaaaattccgtggcctattctaacaattcaatcgccatcccaaccaacttcacaattagcgtgactaccgaaatactgcctgtgagcatgacgaaaaccagcgtagactgcactatgtatatctgtggagactccactgagtgctccaaccttctcctgcagtacggtagcttctgtacccaattgaaccgcgcccttacaggcatcgctgttgagcaagataagaatacccaggaagtttttgcccaggttaagcagatatacaaaacaccgcccattaaggacttcggaggcttcaacttctctcagatactgcctgacccctccaagccatcaaaacgcagcttcattgaggacctcttgttcaacaaagtgactctggctgatgctggcttcattaagcagtacggagattgcctgggagatattgctgccagggacctcatctgcgcccagaagtttaatggcctgacagtcttgcccccacttctgacagacgagatgattgctcagtacacatctgccctcctcgctggcaccataacatccggatggacatttggtgctggtgctgccctccagattcccttcgcaatgcagatggcgtatcgctttaacggcatcggtgtcacacaaaacgtgttgtatgagaaccaaaagctcatcgctaaccagtttaattctgctattggtaagattcaggacagcctgtcatcaaccgcgtctgcccttggtaagttgcaggacgtggtgaaccagaatgctcaggctttgaatactctggtgaagcaactctcttcaaatttcggcgctatctcttctgtgttgaacgacatcctgagtcgccttgataaggtggaagctgaagttcaaattgatagattgattactggcaggctccagtctttgcagacctacgttacacagcagctgattagggcggctgaaattagagcttccgccaatctggctgcaaccaagatgtccgaatgcgtcctgggtcagtcaaagcgcgttgacttttgtggtaaaggctaccacctcatgtcatttccccagtcagcacctcacggagtagtgttcctccacgtcacctacgttccagcacaggaaaagaattttaccactgcgccggcaatctgtcacgacggtaaggcacacttcccccgcgagggcgtattcgtgtctaacggaactcattggttcgtcacacagagaaacttctatgagcctcagatcattaccaccgacaatacatttgtgtccggtaactgcgacgttgtgattggaatcgtcaacaacactgtgtacgatccacttcagccagaactggatagcttcaaggaagaattggacaaatatttcaaaaatcacacttcacccgatgtggacctgggtgacattagtggtatcaatgcgtccgtggtcaatattcaaaaagagattgacaggctcaacgaagtggccaagaacctgaacgaaagtcttatcgatctgcaagaattgggaaagtatgagcagtacatcaagtggccgtggtacatttggttgggttttatcgccggtctgatcgccatcgttatggttaccattatgctttgctgcatgacgagctgttgctcctgtctgaagggatgctgctcttgcggatcatgttgcaagttcgatgaagacgatagcgaaccagttctgaagggcgtcaagctgcattacaca

SEQ ID NO: 12 - DNA, 1764 bp, Severe acute respiratory syndrome coronavirus 2
  source 1..1764: mol_type = unassigned DNA
agtgtggcttctcaaagcattatagcatacactatgtctcttggtgccgaaaattccgtggcctattctaacaattcaatcgccatcccaaccaacttcacaattagcgtgactaccgaaatactgcctgtgagcatgacgaaaaccagcgtagactgcactatgtatatctgtggagactccactgagtgctccaaccttctcctgcagtacggtagcttctgtacccaattgaaccgcgcccttacaggcatcgctgttgagcaagataagaatacccaggaagtttttgcccaggttaagcagatatacaaaacaccgcccattaaggacttcggaggcttcaacttctctcagatactgcctgacccctccaagccatcaaaacgcagcttcattgaggacctcttgttcaacaaagtgactctggctgatgctggcttcattaagcagtacggagattgcctgggagatattgctgccagggacctcatctgcgcccagaagtttaatggcctgacagtcttgcccccacttctgacagacgagatgattgctcagtacacatctgccctcctcgctggcaccataacatccggatggacatttggtgctggtgctgccctccagattcccttcgcaatgcagatggcgtatcgctttaacggcatcggtgtcacacaaaacgtgttgtatgagaaccaaaagctcatcgctaaccagtttaattctgctattggtaagattcaggacagcctgtcatcaaccgcgtctgcccttggtaagttgcaggacgtggtgaaccagaatgctcaggctttgaatactctggtgaagcaactctcttcaaatttcggcgctatctcttctgtgttgaacgacatcctgagtcgccttgatcctccagaagctgaagttcaaattgatagattgattactggcaggctccagtctttgcagacctacgttacacagcagctgattagggcggctgaaattagagcttccgccaatctggctgcaaccaagatgtccgaatgcgtcctgggtcagtcaaagcgcgttgacttttgtggtaaaggctaccacctcatgtcatttccccagtcagcacctcacggagtagtgttcctccacgtcacctacgttccagcacaggaaaagaattttaccactgcgccggcaatctgtcacgacggtaaggcacacttcccccgcgagggcgtattcgtgtctaacggaactcattggttcgtcacacagagaaacttctatgagcctcagatcattaccaccgacaatacatttgtgtccggtaactgcgacgttgtgattggaatcgtcaacaacactgtgtacgatccacttcagccagaactggatagcttcaaggaagaattggacaaatatttcaaaaatcacacttcacccgatgtggacctgggtgacattagtggtatcaatgcgtccgtggtcaatattcaaaaagagattgacaggctcaacgaagtggccaagaacctgaacgaaagtcttatcgatctgcaagaattgggaaagtatgagcagtacatcaagtggccgtggtacatttggttgggttttatcgccggtctgatcgccatcgttatggttaccattatgctttgctgcatgacgagctgttgctcctgtctgaagggatgctgctcttgcggatcatgttgcaagttcgatgaagacgatagcgaaccagttctgaagggcgtcaagctgcattacaca

SEQ ID NO: 13 - DNA, 3816 bp, Severe acute respiratory syndrome coronavirus 2
  source 1..3816: mol_type = unassigned DNA
ttcgttttccttgttctgttgcctctcgttagtagccaatgcgtcaaccttactactagaacccagctccctccagcatataccaactctttcaccaggggcgtatattacccggacaaagtgttccgctcaagtgtgctgcattctacgcaggaccttttcttgccctttttcagtaatgttacttggtttcatgctatccatgtgtctggaactaacggaaccaagcgctttgacaaccccgtcctccctttcaacgatggcgtgtacttcgcttccacggaaaagtcaaacataattcgcggctggatctttggtacaacactcgactcaaagacgcagagcctgctgatcgttaataacgctacaaatgttgtgataaaggtgtgtgaatttcagttctgcaatgatcccttcctgggtgtgtactaccataagaataacaagagctggatggaatccgaatttagggtttacagttccgctaacaactgcacattcgaatacgtaagccagccatttcttatggatcttgagggcaagcaaggaaacttcaagaacttgagggagttcgtgttcaaaaatatcgacggctattttaagatatatagcaagcacactccaataaacttggtgcgcgacctgccccagggattctctgctctggagcccctggtggatctgcccattggaataaacataactcgctttcaaacactgctcgccctgcatcgcagttacctcacccctggtgatagtagttcaggatggacagcaggagccgccgcatactacgtcggctacctgcagcctaggaccttcttgctgaagtacaacgagaacggtacaataactgacgctgtggactgcgctctggaccctctgtccgagacgaagtgcaccctgaagagctttactgttgaaaaaggcatttaccaaaccagcaacttccgcgtccagccaaccgagagcatcgtcagatttcccaacattacaaatctgtgtcccttcggcgaggtgttcaacgccacacgcttcgcttcagtgtacgcatggaaccgcaagcgcatatctaactgcgtcgcggattattctgtcctctacaactccgcctctttctccaccttcaagtgctacggagtgtcaccgactaagctgaacgatctctgctttaccaacgtctacgcggactccttcgtgataagaggtgatgaagtgagacaaatagccccaggtcagactggtaagatcgcagattacaactacaaattgcctgatgatttcactggttgcgttatcgcgtggaactctaataacctcgattctaaggtcggtggtaactacaattacctgtaccgcttgtttaggaagtcaaacctgaagcctttcgagagggatatttcaaccgaaatctatcaagcgggttcaacaccgtgtaacggtgtggaaggatttaactgctacttccccctgcagtcttacggattccagccaaccaatggcgtgggttaccaaccttatcgcgtggtggttctgagtttcgaactgttgcacgctcccgccacggtatgcggtcccaagaagagcactaacttggtgaagaataagtgcgtgaatttcaatttcaatggcctcactggaactggagtgctgaccgaatccaataagaagttcttgcccttccagcagttcggaagagacattgctgacacaaccgacgcggtgcgcgatcctcagactctggagatattggacattacaccatgttctttcggcggtgtgtctgtcattactccgggcacgaatactagcaaccaggtagccgtgctgtaccaagacgtgaattgcacagaggttcccgtcgcaattcacgctgaccagctgacccccacgtggagggtttacagcactggtagtaacgtcttccagacgagagccggttgcttgatcggagcggaacatgtgaataactcctacgagtgcgacatccccatcggagccggtatatgcgcctcttatcagacacaaactaactcacccaggagagcccgcagtgtggcttctcaaagcattatagcatacactatgtctcttggtgccgaaaattccgtggcctattctaacaattcaatcgccatcccaaccaacttcacaattagcgtgactaccgaaatactgcctgtgagcatgacgaaaaccagcgtagactgcactatgtatatctgtggagactccactgagtgctccaaccttctcctgcagtacggtagcttctgtacccaattgaaccgcgcccttacaggcatcgctgttgagcaagataagaatacccaggaagtttttgcccaggttaagcagatatacaaaacaccgcccattaaggacttcggaggcttcaacttctctcagatactgcctgacccctccaagccatcaaaacgcagcttcattgaggacctcttgttcaacaaagtgactctggctgatgctggcttcattaagcagtacggagattgcctgggagatattgctgccagggacctcatctgcgcccagaagtttaatggcctgacagtcttgcccccacttctgacagacgagatgattgctcagtacacatctgccctcctcgctggcaccataacatccggatggacatttggtgctggtgctgccctccagattcccttcgcaatgcagatggcgtatcgctttaacggcatcggtgtcacacaaaacgtgttgtatgagaaccaaaagctcatcgctaaccagtttaattctgctattggtaagattcaggacagcctgtcatcaaccgcgtctgcccttggtaagttgcaggacgtggtgaaccagaatgctcaggctttgaatactctggtgaagcaactctcttcaaatttcggcgctatctcttctgtgttgaacgacatcctgagtcgccttgataaggtggaagctgaagttcaaattgatagattgattactggcaggctccagtctttgcagacctacgttacacagcagctgattagggcggctgaaattagagcttccgccaatctggctgcaaccaagatgtccgaatgcgtcctgggtcagtcaaagcgcgttgacttttgtggtaaaggctaccacctcatgtcatttccccagtcagcacctcacggagtagtgttcctccacgtcacctacgttccagcacaggaaaagaattttaccactgcgccggcaatctgtcacgacggtaaggcacacttcccccgcgagggcgtattcgtgtctaacggaactcattggttcgtcacacagagaaacttctatgagcctcagatcattaccaccgacaatacatttgtgtccggtaactgcgacgttgtgattggaatcgtcaacaacactgtgtacgatccacttcagccagaactggatagcttcaaggaagaattggacaaatatttcaaaaatcacacttcacccgatgtggacctgggtgacattagtggtatcaatgcgtccgtggtcaatattcaaaaagagattgacaggctcaacgaagtggccaagaacctgaacgaaagtcttatcgatctgcaagaattgggaaagtatgagcagtacatcaagtggccgtggtacatttggttgggttttatcgccggtctgatcgccatcgttatggttaccattatgctttgctgcatgacgagctgttgctcctgtctgaagggatgctgctcttgcggatcatgttgcaagttcgatgaagacgatagcgaaccagttctgaagggcgtcaagctgcattacaca

SEQ ID NO: 14 - DNA, 3804 bp, Severe acute respiratory syndrome coronavirus 2
  source 1..3804: mol_type = unassigned DNA
ttcgttttccttgttctgttgcctctcgttagtagccaatgcgtcaaccttactactagaacccagctccctccagcatataccaactctttcaccaggggcgtatattacccggacaaagtgttccgctcaagtgtgctgcattctacgcaggaccttttcttgccctttttcagtaatgttacttggtttcatgctatccatgtgtctggaactaacggaaccaagcgctttgacaaccccgtcctccctttcaacgatggcgtgtacttcgcttccacggaaaagtcaaacataattcgcggctggatctttggtacaacactcgactcaaagacgcagagcctgctgatcgttaataacgctacaaatgttgtgataaaggtgtgtgaatttcagttctgcaatgatcccttcctgggtgtgtactaccataagaataacaagagctggatggaatccgaatttagggtttacagttccgctaacaactgcacattcgaatacgtaagccagccatttcttatggatcttgagggcaagcaaggaaacttcaagaacttgagggagttcgtgttcaaaaatatcgacggctattttaagatatatagcaagcacactccaataaacttggtgcgcgacctgccccagggattctctgctctggagcccctggtggatctgcccattggaataaacataactcgctttcaaacactgctcgccctgcatcgcagttacctcacccctggtgatagtagttcaggatggacagcaggagccgccgcatactacgtcggctacctgcagcctaggaccttcttgctgaagtacaacgagaacggtacaataactgacgctgtggactgcgctctggaccctctgtccgagacgaagtgcaccctgaagagctttactgttgaaaaaggcatttaccaaaccagcaacttccgcgtccagccaaccgagagcatcgtcagatttcccaacattacaaatctgtgtcccttcggcgaggtgttcaacgccacacgcttcgcttcagtgtacgcatggaaccgcaagcgcatatctaactgcgtcgcggattattctgtcctctacaactccgcctctttctccaccttcaagtgctacggagtgtcaccgactaagctgaacgatctctgctttaccaacgtctacgcggactccttcgtgataagaggtgatgaagtgagacaaatagccccaggtcagactggtaagatcgcagattacaactacaaattgcctgatgatttcactggttgcgttatcgcgtggaactctaataacctcgattctaaggtcggtggtaactacaattacctgtaccgcttgtttaggaagtcaaacctgaagcctttcgagagggatatttcaaccgaaatctatcaagcgggttcaacaccgtgtaacggtgtggaaggatttaactgctacttccccctgcagtcttacggattccagccaaccaatggcgtgggttaccaaccttatcgcgtggtggttctgagtttcgaactgttgcacgctcccgccacggtatgcggtcccaagaagagcactaacttggtgaagaataagtgcgtgaatttcaatttcaatggcctcactggaactggagtgctgaccgaatccaataagaagttcttgcccttccagcagttcggaagagacattgctgacacaaccgacgcggtgcgcgatcctcagactctggagatattggacattacaccatgttctttcggcggtgtgtctgtcattactccgggcacgaatactagcaaccaggtagccgtgctgtaccaagacgtgaattgcacagaggttcccgtcgcaattcacgctgaccagctgacccccacgtggagggtttacagcactggtagtaacgtcttccagacgagagccggttgcttgatcggagcggaacatgtgaataactcctacgagtgcgacatccccatcggagccggtatatgcgcctcttatcagacacaaactaactcacgcagtgtggcttctcaaagcattatagcatacactatgtctcttggtgccgaaaattccgtggcctattctaacaattcaatcgccatcccaaccaacttcacaattagcgtgactaccgaaatactgcctgtgagcatgacgaaaaccagcgtagactgcactatgtatatctgtggagactccactgagtgctccaaccttctcctgcagtacggtagcttctgtacccaattgaaccgcgcccttacaggcatcgctgttgagcaagataagaatacccaggaagtttttgcccaggttaagcagatatacaaaacaccgcccattaaggacttcggaggcttcaacttctctcagatactgcctgacccctccaagccatcaaaacgcagcttcattgaggacctcttgttcaacaaagtgactctggctgatgctggcttcattaagcagtacggagattgcctgggagatattgctgccagggacctcatctgcgcccagaagtttaatggcctgacagtcttgcccccacttctgacagacgagatgattgctcagtacacatctgccctcctcgctggcaccataacatccggatggacatttggtgctggtgctgccctccagattcccttcgcaatgcagatggcgtatcgctttaacggcatcggtgtcacacaaaacgtgttgtatgagaaccaaaagctcatcgctaaccagtttaattctgctattggtaagattcaggacagcctgtcatcaaccgcgtctgcccttggtaagttgcaggacgtggtgaaccagaatgctcaggctttgaatactctggtgaagcaactctcttcaaatttcggcgctatctcttctgtgttgaacgacatcctgagtcgccttgataaggtggaagctgaagttcaaattgatagattgattactggcaggctccagtctttgcagacctacgttacacagcagctgattagggcggctgaaattagagcttccgccaatctggctgcaaccaagatgtccgaatgcgtcctgggtcagtcaaagcgcgttgacttttgtggtaaaggctaccacctcatgtcatttccccagtcagcacctcacggagtagtgttcctccacgtcacctacgttccagcacaggaaaagaattttaccactgcgccggcaatctgtcacgacggtaaggcacacttcccccgcgagggcgtattcgtgtctaacggaactcattggttcgtcacacagagaaacttctatgagcctcagatcattaccaccgacaatacatttgtgtccggtaactgcgacgttgtgattggaatcgtcaacaacactgtgtacgatccacttcagccagaactggatagcttcaaggaagaattggacaaatatttcaaaaatcacacttcacccgatgtggacctgggtgacattagtggtatcaatgcgtccgtggtcaatattcaaaaagagattgacaggctcaacgaagtggccaagaacctgaacgaaagtcttatcgatctgcaagaattgggaaagtatgagcagtacatcaagtggccgtggtacatttggttgggttttatcgccggtctgatcgccatcgttatggttaccattatgctttgctgcatgacgagctgttgctcctgtctgaagggatgctgctcttgcggatcatgttgcaagttcgatgaagacgatagcgaaccagttctgaagggcgtcaagctgcattacaca

SEQ ID NO: 15 - DNA, 3804 bp, Severe acute respiratory syndrome coronavirus 2
  source 1..3804: mol_type = unassigned DNA
ttcgttttccttgttctgttgcctctcgttagtagccaatgcgtcaaccttactactagaacccagctccctccagcatataccaactctttcaccaggggcgtatattacccggacaaagtgttccgctcaagtgtgctgcattctacgcaggaccttttcttgccctttttcagtaatgttacttggtttcatgctatccatgtgtctggaactaacggaaccaagcgctttgacaaccccgtcctccctttcaacgatggcgtgtacttcgcttccacggaaaagtcaaacataattcgcggctggatctttggtacaacactcgactcaaagacgcagagcctgctgatcgttaataacgctacaaatgttgtgataaaggtgtgtgaatttcagttctgcaatgatcccttcctgggtgtgtactaccataagaataacaagagctggatggaatccgaatttagggtttacagttccgctaacaactgcacattcgaatacgtaagccagccatttcttatggatcttgagggcaagcaaggaaacttcaagaacttgagggagttcgtgttcaaaaatatcgacggctattttaagatatatagcaagcacactccaataaacttggtgcgcgacctgccccagggattctctgctctggagcccctggtggatctgcccattggaataaacataactcgctttcaaacactgctcgccctgcatcgcagttacctcacccctggtgatagtagttcaggatggacagcaggagccgccgcatactacgtcggctacctgcagcctaggaccttcttgctgaagtacaacgagaacggtacaataactgacgctgtggactgcgctctggaccctctgtccgagacgaagtgcaccctgaagagctttactgttgaaaaaggcatttaccaaaccagcaacttccgcgtccagccaaccgagagcatcgtcagatttcccaacattacaaatctgtgtcccttcggcgaggtgttcaacgccacacgcttcgcttcagtgtacgcatggaaccgcaagcgcatatctaactgcgtcgcggattattctgtcctctacaactccgcctctttctccaccttcaagtgctacggagtgtcaccgactaagctgaacgatctctgctttaccaacgtctacgcggactccttcgtgataagaggtgatgaagtgagacaaatagccccaggtcagactggtaagatcgcagattacaactacaaattgcctgatgatttcactggttgcgttatcgcgtggaactctaataacctcgattctaaggtcggtggtaactacaattacctgtaccgcttgtttaggaagtcaaacctgaagcctttcgagagggatatttcaaccgaaatctatcaagcgggttcaacaccgtgtaacggtgtggaaggatttaactgctacttccccctgcagtcttacggattccagccaaccaatggcgtgggttaccaaccttatcgcgtggtggttctgagtttcgaactgttgcacgctcccgccacggtatgcggtcccaagaagagcactaacttggtgaagaataagtgcgtgaatttcaatttcaatggcctcactggaactggagtgctgaccgaatccaataagaagttcttgcccttccagcagttcggaagagacattgctgacacaaccgacgcggtgcgcgatcctcagactctggagatattggacattacaccatgttctttcggcggtgtgtctgtcattactccgggcacgaatactagcaaccaggtagccgtgctgtaccaagacgtgaattgcacagaggttcccgtcgcaattcacgctgaccagctgacccccacgtggagggtttacagcactggtagtaacgtcttccagacgagagccggttgcttgatcggagcggaacatgtgaataactcctacgagtgcgacatccccatcggagccggtatatgcgcctcttatcagacacaaactaactcacgcagtgtggcttctcaaagcattatagcatacactatgtctcttggtgccgaaaattccgtggcctattctaacaattcaatcgccatcccaaccaacttcacaattagcgtgactaccgaaatactgcctgtgagcatgacgaaaaccagcgtagactgcactatgtatatctgtggagactccactgagtgctccaaccttctcctgcagtacggtagcttctgtacccaattgaaccgcgcccttacaggcatcgctgttgagcaagataagaatacccaggaagtttttgcccaggttaagcagatatacaaaacaccgcccattaaggacttcggaggcttcaacttctctcagatactgcctgacccctccaagccatcaaaacgcagcttcattgaggacctcttgttcaacaaagtgactctggctgatgctggcttcattaagcagtacggagattgcctgggagatattgctgccagggacctcatctgcgcccagaagtttaatggcctgacagtcttgcccccacttctgacagacgagatgattgctcagtacacatctgccctcctcgctggcaccataacatccggatggacatttggtgctggtgctgccctccagattcccttcgcaatgcagatggcgtatcgctttaacggcatcggtgtcacacaaaacgtgttgtatgagaaccaaaagctcatcgctaaccagtttaattctgctattggtaagattcaggacagcctgtcatcaaccgcgtctgcccttggtaagttgcaggacgtggtgaaccagaatgctcaggctttgaatactctggtgaagcaactctcttcaaatttcggcgctatctcttctgtgttgaacgacatcctgagtcgccttgatcctccagaagctgaagttcaaattgatagattgattactggcaggctccagtctttgcagacctacgttacacagcagctgattagggcggctgaaattagagcttccgccaatctggctgcaaccaagatgtccgaatgcgtcctgggtcagtcaaagcgcgttgacttttgtggtaaaggctaccacctcatgtcatttccccagtcagcacctcacggagtagtgttcctccacgtcacctacgttccagcacaggaaaagaattttaccactgcgccggcaatctgtcacgacggtaaggcacacttcccccgcgagggcgtattcgtgtctaacggaactcattggttcgtcacacagagaaacttctatgagcctcagatcattaccaccgacaatacatttgtgtccggtaactgcgacgttgtgattggaatcgtcaacaacactgtgtacgatccacttcagccagaactggatagcttcaaggaagaattggacaaatatttcaaaaatcacacttcacccgatgtggacctgggtgacattagtggtatcaatgcgtccgtggtcaatattcaaaaagagattgacaggctcaacgaagtggccaagaacctgaacgaaagtcttatcgatctgcaagaattgggaaagtatgagcagtacatcaagtggccgtggtacatttggttgggttttatcgccggtctgatcgccatcgttatggttaccattatgctttgctgcatgacgagctgttgctcctgtctgaagggatgctgctcttgcggatcatgttgcaagttcgatgaagacgatagcgaaccagttctgaagggcgtcaagctgcattacaca

SEQ ID NO: 16 - protein, 31 aa, synthetic construct
DAMKRGLCCVLLLCGAVFVSPSQEIHARFRR

SEQ ID NO: 17 - protein, 17 aa, synthetic construct
DWTWILFLVAAATRVHS

SEQ ID NO: 18 - protein, 223 aa, Severe acute respiratory syndrome coronavirus 2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNF

SEQ ID NO: 19 - protein, 223 aa, Severe acute respiratory syndrome coronavirus 2
RVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNF

SEQ ID NO: 20 - DNA, 669 bp, Severe acute respiratory syndrome coronavirus 2
  source 1..669: mol_type = unassigned DNA
cgcgtccagccaaccgagagcatcgtcagatttcccaacattacaaatctgtgtcccttcggcgaggtgttcaacgccacacgcttcgcttcagtgtacgcatggaaccgcaagcgcatatctaactgcgtcgcggattattctgtcctctacaactccgcctctttctccaccttcaagtgctacggagtgtcaccgactaagctgaacgatctctgctttaccaacgtctacgcggactccttcgtgataagaggtgatgaagtgagacaaatagccccaggtcagactggtaagatcgcagattacaactacaaattgcctgatgatttcactggttgcgttatcgcgtggaactctaataacctcgattctaaggtcggtggtaactacaattacaggtaccgcttgtttaggaagtcaaacctgaagcctttcgagagggatatttcaaccgaaatctatcaagcgggttcaaagccgtgtaacggtgtggaaggatttaactgctacttccccctgcagtcttacggattccagccaaccaatggcgtgggttaccaaccttatcgcgtggtggttctgagtttcgaactgttgcacgctcccgccacggtatgcggtcccaagaagagcactaacttggtgaagaataagtgcgtgaatttc

SEQ ID NO: 21 - DNA, 669 bp, Severe acute respiratory syndrome coronavirus 2
  source 1..669: mol_type = unassigned DNA
cgcgtccagccaaccgagagcatcgtcagatttcccaacattacaaatctgtgtcccttcgacgaggtgttcaacgccacacgcttcgcttcagtgtacgcatggaaccgcaagcgcatatctaactgcgtcgcggattattctgtcctctacaacctcgcccctttcttcaccttcaagtgctacggagtgtcaccgactaagctgaacgatctctgctttaccaacgtctacgcggactccttcgtgataagaggtgatgaagtgagacaaatagccccaggtcagactggtaacatcgcagattacaactacaaattgcctgatgatttcactggttgcgttatcgcgtggaactctaataagctcgattctaaggtcagtggtaactacaattacctgtaccgcttgtttaggaagtcaaacctgaagcctttcgagagggatatttcaaccgaaatctatcaagcgggtaacaagccgtgtaacggtgtggcaggatttaactgctacttccccctgcggtcttacagcttccggccaacctatggcgtgggtcaccaaccttatcgcgtggtggttctgagtttcgaactgttgcacgctcccgccacggtatgcggtcccaagaagagcactaacttggtgaagaataagtgcgtgaatttc

SEQ ID NO: 22 - skipped

SEQ ID NO: 23 - DNA, 51 bp, synthetic construct
  source 1..51: mol_type = other DNA
gaaaaacaaaaaacaaaaaaaacaaaaaaaaaaccaaaaaaacaaaacaca

SEQ ID NO: 24 - DNA, 27 bp, synthetic construct
  source 1..27: mol_type = other DNA
acgagtcctggactgaaacggacttgt

SEQ ID NO: 25 - RNA, 52 nt, synthetic construct
  source 1..52: mol_type = other RNA
aaaatccgttgaccttaaacggtcgtgtgggttcaagtccctccacccccac

SEQ ID NO: 26 - RNA, 16 nt, synthetic construct
  source 1..16: mol_type = other RNA
gagacgctacggactt

SEQ ID NO: 27 - RNA, 33 nt, synthetic construct
  source 1..33: mol_type = other RNA
gggagaccctcgaccgtcgattgtccactggtc

SEQ ID NO: 28 - RNA, 35 nt, synthetic construct
  source 1..35: mol_type = other RNA
accagtggacaatcgacggataacagcatatctag

SEQ ID NO: 29 - RNA, 19 nt, synthetic construct
  source 1..19: mol_type = other RNA
taatacgactcactatagg

SEQ ID NO: 30 - RNA, 54 nt, synthetic construct
  source 1..54: mol_type = other RNA
gagggcagaggaagtcttctaacatgcggtgacgtggaggagaatcccggccct

SEQ ID NO: 31 - RNA, 57 nt, synthetic construct
  source 1..57: mol_type = other RNA
gctactaacttcagcctgctgaagcaggctggagacgtggaggagaaccctggacct

SEQ ID NO: 32 - RNA, 131 nt, synthetic construct
  source 1..131: mol_type = other RNA
aacaatagatgacttacaactaatcggaaggtgcagagactcgacgggagctaccctaacgtcaagacgagggtaaagagagagtccaattctcaaagccaataggcagtagcgaaagctgcaagagaatg

SEQ ID NO: 33 - RNA, 116 nt, synthetic construct
  source 1..116: mol_type = other RNA
aaataattgagccttaaagaagaaattctttaagtggatgctctcaaactcagggaaacctaaatctagttatagacaaggcaatcctgagccaagccgaagtagtaattagtaag

SEQ ID NO: 34 - DNA, 3819 bp, Severe acute respiratory syndrome coronavirus 2
  source 1..3819: mol_type = unassigned DNA
atgttcgttttccttgttctgttgcctctcgttagtagccaatgcgtcaaccttactactagaacccagctccctccagcatataccaactctttcaccaggggcgtatattacccggacaaagtgttccgctcaagtgtgctgcattctacgcaggaccttttcttgccctttttcagtaatgttacttggtttcatgctatccatgtgtctggaactaacggaaccaagcgctttgacaaccccgtcctccctttcaacgatggcgtgtacttcgcttccacggaaaagtcaaacataattcgcggctggatctttggtacaacactcgactcaaagacgcagagcctgctgatcgttaataacgctacaaatgttgtgataaaggtgtgtgaatttcagttctgcaatgatcccttcctgggtgtgtactaccataagaataacaagagctggatggaatccgaatttagggtttacagttccgctaacaactgcacattcgaatacgtaagccagccatttcttatggatcttgagggcaagcaaggaaacttcaagaacttgagggagttcgtgttcaaaaatatcgacggctattttaagatatatagcaagcacactccaataaacttggtgcgcgacctgccccagggattctctgctctggagcccctggtggatctgcccattggaataaacataactcgctttcaaacactgctcgccctgcatcgcagttacctcacccctggtgatagtagttcaggatggacagcaggagccgccgcatactacgtcggctacctgcagcctaggaccttcttgctgaagtacaacgagaacggtacaataactgacgctgtggactgcgctctggaccctctgtccgagacgaagtgcaccctgaagagctttactgttgaaaaaggcatttaccaaaccagcaacttccgcgtccagccaaccgagagcatcgtcagatttcccaacattacaaatctgtgtcccttcggcgaggtgttcaacgccacacgcttcgcttcagtgtacgcatggaaccgcaagcgcatatctaactgcgtcgcggattattctgtcctctacaactccgcctctttctccaccttcaagtgctacggagtgtcaccgactaagctgaacgatctctgctttaccaacgtctacgcggactccttcgtgataagaggtgatgaagtgagacaaatagccccaggtcagactggtaagatcgcagattacaactacaaattgcctgatgatttcactggttgcgttatcgcgtggaactctaataacctcgattctaaggtcggtggtaactacaattacctgtaccgcttgtttaggaagtcaaacctgaagcctttcgagagggatatttcaaccgaaatctatcaagcgggttcaacaccgtgtaacggtgtggaaggatttaactgctacttccccctgcagtcttacggattccagccaaccaatggcgtgggttaccaaccttatcgcgtggtggttctgagtttcgaactgttgcacgctcccgccacggtatgcggtcccaagaagagcactaacttggtgaagaataagtgcgtgaatttcaatttcaatggcctcactggaactggagtgctgaccgaatccaataagaagttcttgcccttccagcagttcggaagagacattgctgacacaaccgacgcggtgcgcgatcctcagactctggagatattggacattacaccatgttctttcggcggtgtgtctgtcattactccgggcacgaatactagcaaccaggtagccgtgctgtaccaagacgtgaattgcacagaggttcccgtcgcaattcacgctgaccagctgacccccacgtggagggtttacagcactggtagtaacgtcttccagacgagagccggttgcttgatcggagcggaacatgtgaataactcctacgagtgcgacatccccatcggagccggtatatgcgcctcttatcagacacaaactaactcacccaggagagcccgcagtgtggcttctcaaagcattatagcatacactatgtctcttggtgccgaaaattccgtggcctattctaacaattcaatcgccatcccaaccaacttcacaattagcgtgactaccgaaatactgcctgtgagcatgacgaaaaccagcgtagactgcactatgtatatctgtggagactccactgagtgctccaaccttctcctgcagtacggtagcttctgtacccaattgaaccgcgcccttacaggcatcgctgttgagcaagataagaatacccaggaagtttttgcccaggttaagcagatatacaaaacaccgcccattaaggacttcggaggcttcaacttctctcagatactgcctgacccctccaagccatcaaaacgcagcttcattgaggacctcttgttcaacaaagtgactctggctgatgctggcttcattaagcagtacggagattgcctgggagatattgctgccagggacctcatctgcgcccagaagtttaatggcctgacagtcttgcccccacttctgacagacgagatgattgctcagtacacatctgccctcctcgctggcaccataacatccggatggacatttggtgctggtgctgccctccagattcccttcgcaatgcagatggcgtatcgctttaacggcatcggtgtcacacaaaacgtgttgtatgagaaccaaaagctcatcgctaaccagtttaattctgctattggtaagattcaggacagcctgtcatcaaccgcgtctgcccttggtaagttgcaggacgtggtgaaccagaatgctcaggctttgaatactctggtgaagcaactctcttcaaatttcggcgctatctcttctgtgttgaacgacatcctgagtcgccttgataaggtggaagctgaagttcaaattgatagattgattactggcaggctccagtctttgcagacctacgttacacagcagctgattagggcggctgaaattagagcttccgccaatctggctgcaaccaagatgtccgaatgcgtcctgggtcagtcaaagcgcgttgacttttgtggtaaaggctaccacctcatgtcatttccccagtcagcacctcacggagtagtgttcctccacgtcacctacgttccagcacaggaaaagaattttaccactgcgccggcaatctgtcacgacggtaaggcacacttcccccgcgagggcgtattcgtgtctaacggaactcattggttcgtcacacagagaaacttctatgagcctcagatcattaccaccgacaatacatttgtgtccggtaactgcgacgttgtgattggaatcgtcaacaacactgtgtacgatccacttcagccagaactggatagcttcaaggaagaattggacaaatatttcaaaaatcacacttcacccgatgtggacctgggtgacattagtggtatcaatgcgtccgtggtcaatattcaaaaagagattgacaggctcaacgaagtggccaagaacctgaacgaaagtcttatcgatctgcaagaattgggaaagtatgagcagtacatcaagtggccgtggtacatttggttgggttttatcgccggtctgatcgccatcgttatggttaccattatgctttgctgcatgacgagctgttgctcctgtctgaagggatgctgctcttgcggatcatgttgcaagttcgatgaagacgatagcgaaccagttctgaagggcgtcaagctgcattacaca

SEQ ID NO: 35 - DNA, 669 bp, Severe acute respiratory syndrome coronavirus 2
  source 1..669: mol_type = unassigned DNA
cgcgtccagccaaccgagagcatcgtcagatttcccaacattacaaatctgtgtcccttcggcgaggtgttcaacgccacacgcttcgcttcagtgtacgcatggaaccgcaagcgcatatctaactgcgtcgcggattattctgtcctctacaactccgcctctttctccaccttcaagtgctacggagtgtcaccgactaagctgaacgatctctgctttaccaacgtctacgcggactccttcgtgataagaggtgatgaagtgagacaaatagccccaggtcagactggtaagatcgcagattacaactacaaattgcctgatgatttcactggttgcgttatcgcgtggaactctaataacctcgattctaaggtcggtggtaactacaattacctgtaccgcttgtttaggaagtcaaacctgaagcctttcgagagggatatttcaaccgaaatctatcaagcgggttcaacaccgtgtaacggtgtggaaggatttaactgctacttccccctgcagtcttacggattccagccaaccaatggcgtgggttaccaaccttatcgcgtggtggttctgagtttcgaactgttgcacgctcccgccacggtatgcggtcccaagaagagcactaacttggtgaagaataagtgcgtgaatttc

SEQ ID NO: 36 - DNA, 96 bp, synthetic construct
  source 1..96: mol_type = other DNA
ggaagcggctacatcccagaagcccctagagacggacaggcttacgtgcgaaaagacggcgagtgggtgctgctgagcacattcctgggaaggagc

SEQ ID NO: 37 - DNA, 99 bp, synthetic construct
  source 1..99: mol_type = other DNA
cgaatgaagcagattgaggataaaattgaggagattctcagcaaaatttaccacatagaaaatgagatcgctcggattaaaaaactgatcggagaaaga

SEQ ID NO: 38 - DNA, 30 bp, synthetic construct
  source 1..30: mol_type = other DNA
ggcggaggaggcagcggcggaggaggcagc

SEQ ID NO: 39 - DNA, 741 bp, Coxsackievirus B3
  source 1..741: mol_type = unassigned DNA
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 40 - protein, 1272 aa, Severe acute respiratory syndrome coronavirus 2
FVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 41 - protein, 223 aa, Severe acute respiratory syndrome coronavirus 2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNF

SEQ ID NO: 42 - DNA, 669 bp, Severe acute respiratory syndrome coronavirus 2
  source 1..669: mol_type = unassigned DNA
cgcgtccagccaaccgagagcatcgtcagatttcccaacattacaaatctgtgtcccttcggcgaggtgttcaacgccacacgcttcgcttcagtgtacgcatggaaccgcaagcgcatatctaactgcgtcgcggattattctgtcctctacaactccgcctctttctccaccttcaagtgctacggagtgtcaccgactaagctgaacgatctctgctttaccaacgtctacgcggactccttcgtgataagaggtgatgaagtgagacaaatagccccaggtcagactggtaacatcgcagattacaactacaaattgcctgatgatttcactggttgcgttatcgcgtggaactctaataacctcgattctaaggtcggtggtaactacaattacctgtaccgcttgtttaggaagtcaaacctgaagcctttcgagagggatatttcaaccgaaatctatcaagcgggttcaacaccgtgtaacggtgtgaaaggatttaactgctacttccccctgcagtcttacggattccagccaacctatggcgtgggttaccaaccttatcgcgtggtggttctgagtttcgaactgttgcacgctcccgccacggtatgcggtcccaagaagagcactaacttggtgaagaataagtgcgtgaatttc

SEQ ID NO: 43 - protein, 18 aa, synthetic construct
GIYQTSNFRVQPTESIVR

SEQ ID NO: 44 - protein, 17 aa, synthetic construct
RVQPTESIVRFPNITNL

SEQ ID NO: 45 - protein, 17 aa, synthetic construct
IVRFPNITNLCPFGEVF

SEQ ID NO: 46 - protein, 18 aa, synthetic construct
TNLCPFGEVFNATRFASV

SEQ ID NO: 47 - protein, 18 aa, synthetic construct
VFNATRFASVYAWNRKRI

SEQ ID NO: 48 - protein, 17 aa, synthetic construct
SVYAWNRKRISNCVADY

SEQ ID NO: 49 - protein, 17 aa, synthetic construct
KRISNCVADYSVLYNSA

SEQ ID NO: 50 - protein, 18 aa, synthetic construct
ADYSVLYNSASFSTFKCY

SEQ ID NO: 51 - protein, 17 aa, synthetic construct
SASFSTFKCYGVSPTKL

SEQ ID NO: 52 - protein, 18 aa, synthetic construct
KCYGVSPTKLNDLCFTNV

SEQ ID NO: 53 - protein, 18 aa, synthetic construct
KLNDLCFTNVYADSFVIR

SEQ ID NO: 54 - protein, 18 aa, synthetic construct
NVYADSFVIRGDEVRQIA

SEQ ID NO: 55 - protein, 18 aa, synthetic construct
IRGDEVRQIAPGQTGKIA

SEQ ID NO: 56 - protein, 16 aa, synthetic construct
IAPGQTGKIADYNYKL

SEQ ID NO: 57 - protein, 18 aa, synthetic construct
GKIADYNYKLPDDFTGCV

SEQ ID NO: 58 - protein, 18 aa, synthetic construct
KLPDDFTGCVIAWNSNNL

SEQ ID NO: 59 - protein, 18 aa, synthetic construct
CVIAWNSNNLDSKVGGNY

SEQ ID NO: 60 - protein, 18 aa, synthetic construct
NLDSKVGGNYNYRYRLFR

SEQ ID NO: 61 - protein, 17 aa, synthetic construct
NYNYRYRLFRKSNLKPF

SEQ ID NO: 62 - protein, 18 aa, synthetic construct
LFRKSNLKPFERDISTEI

SEQ ID NO: 63 - protein, 13 aa, synthetic construct
PFERDISTEIYQA

SEQ ID NO: 64 - protein, 18 aa, synthetic construct
RDISTEIYQAGSKPCNGV

SEQ ID NO: 65 - protein, 18 aa, synthetic construct
YQAGSKPCNGVEGFNCYF

SEQ ID NO: 66 - protein, 17 aa, synthetic construct
NGVEGFNCYFPLQSYGF

SEQ ID NO: 67 - protein, 18 aa, synthetic construct
CYFPLQSYGFQPTNGVGY

SEQ ID NO: 68 - protein, 18 aa, synthetic construct
GFQPTNGVGYQPYRVVVL

SEQ ID NO: 69 - protein, 17 aa, synthetic construct
GYQPYRVVVLSFELLHA

SEQ ID NO: 70 - protein, 18 aa, synthetic construct
VVLSFELLHAPATVCGPK

SEQ ID NO: 71 - protein, 17 aa, synthetic construct
HAPATVCGPKKSTNLVK

SEQ ID NO: 72 - protein, 18 aa, synthetic construct
GPKKSTNLVKNKCVNFNF

SEQ ID NO: 73 - protein, 18 aa, synthetic construct
VKNKCVNFNFNGLTGTGV

(19) United States
(12) Patent Application Publication
(10) Pub. No.: US 2025/0082746 A1
(43) Pub. Date: Mar. 13, 2025

(54) CIRCULAR RNA VACCINES AGAINST SARS-COV-2 VARIANTS AND METHODS OF USE THEREOF

(71) Applicant: Peking University, Beijing (CN)

(72) Inventors: Wensheng WEI, Beijing (CN); Liang QU, Beijing (CN); Zongyi YI, Beijing (CN); Yong SHEN, Beijing (CN); Liangru LIN, Beijing (CN); Feng CHEN, Beijing (CN)

(73) Assignee: Peking University, Beijing (CN)

(21) Appl. No.: 18/727,308

(22) PCT Filed: Jan. 9, 2023
(86) PCT No.: PCT/CN2023/071196

§ 371 Date: Jul. 8, 2024

(30) Foreign Application Priority Data
Jan. 11, 2022 (WO) PCT/CN2022/071347
Jan. 28, 2022 (WO) PCT/CN2022/074752

(51) Int. Cl. A61K 39/215; A61K 39/00; A61P 37/04; C07K 14/005

(52) U.S. Cl. CPC A61K 39/215; A61P 37/04; C07K 14/005; A61K 2039/53; C12N 2770/20022; C12N 2770/20034

(57) ABSTRACT

Provided are circular RNAs (circRNAs) encoding an antigenic polypeptide of a SARS-CoV-2 variant. Provided are circRNA vaccines against a SARS-CoV-2 variant, such as a Delta or Omicron variant. The circRNA vaccine comprises a circRNA comprising a nucleic acid sequence encoding an antigenic polypeptide comprising a Spike(S) protein or a fragment thereof of a SRAS-CoV-2 variant. Also provided are methods of treating or preventing a SARS-CoV-2 infection using the circRNAs or compositions thereof.

### CROSS-REFERENCE TO RELATED APPLICATIONS

This application claims priority to International Patent Application Nos. PCT/CN2022/071347 filed Jan. 11, 2022 and PCT/CN2022/074752 filed Jan. 28, 2022, the disclosure of which is hereby incorporated by reference in its entirety.

### REFERENCE TO AN ELECTRONIC SEQUENCE LISTING

The contents of the electronic sequence listing (165392001043seqlist.xml; Size: 95,801 bytes; and Date of Creation: Jan. 6, 2023) is herein incorporated by reference in its entirety.

### FIELD OF THE INVENTION

The present application relates to circular RNA (circRNA) vaccines against a SARS-Co-V-2 variant, and methods of use thereof.

### BACKGROUND

Coronavirus disease 2019 (COVID-19) is a serious worldwide public health emergency caused by a novel severe acute respiratory syndrome coronavirus (SARS-CoV-2)^{1,2}. To date, COVID-19 has resulted in more than 2 hundred million confirmed cases and over 5 million confirmed deaths (World Health Organization). Thus, there is an urgent need for the development of safe and effective vaccines against SARS-CoV-2 and emerging variants.

SARS-CoV-2, together with Severe Acute Respiratory Syndrome (SARS)-CoV and Middle East Respiratory Syndrome (MERS)-CoV, other two highly pathogenic coronaviruses, belongs to the genus Betacoronavirus of the Coronaviridae family³. SARS-CoV-2 is a single-strand, positive-sense, enveloped virus, and its virion is composed of an inner capsid formed by 30-kb RNA genome wrapped by the nucleocapsid (N) proteins and a lipid envelope coated with the membrane (M), envelope (E), and trimeric spike(S) proteins⁴. The S protein of SARS-CoV-2, composed of S1 and S2 subunits, is the major surface protein of the virion. The S protein mediates viral entry into host cells by binding to its receptor, angiotensin-converting enzyme 2 (ACE2), through the receptor-binding domain (RBD) at the C terminus of the S1 subunit. This binding subsequently induces the fusion between the SARS-CoV-2 envelope and the host cell membrane mediated by the S2 subunit, leading to the release of the viral genome into the cytoplasm⁵⁻⁸.

The S protein, S1 subunit, or the RBD antigen of SARS-CoV-2, could induce both B cell and T cell responses, generating highly potent neutralizing antibodies against SARS-CoV-2⁹⁻¹¹. Vaccination is the most promising approach to end COVID-19 pandemic. Traditional vaccine platforms, such as inactivated vaccines, virus-like particle vaccines, and viral vector-based vaccines have been adopted to develop SARS-CoV-2 vaccines¹²⁻²⁰. Importantly, the mRNA vaccines against SARS-CoV-2 have been developed at warp speed and urgently approved for use²¹⁻²⁷, despite the fact that such strategy had never been applied commercially before²⁸. The mRNA vaccine contains a linear single-strand RNA, consisting of 5′ cap, the untranslated region (UTR), antigen-coding region, and 3′ polyA tail, which is delivered into bodies via lipid-nano particle (LNP) encapsulation²⁸. The clinical-scale mRNA vaccines could be manufactured rapidly upon the viral antigen sequence is released²¹. However, the current mRNA vaccine still has certain limitations due to its inherent instability and suboptimal thermostability after LNP encapsulation for in vivo administration²⁹⁻³¹, as well as potential immunogenic side effects^{32,33}.

Circular RNAs (circRNAs) are covalently closed single-stranded RNA transcripts, comprising a large class of non-coding RNAs generated by a non-canonical RNA splicing event called backsplicing in eukaryotic cells³⁴⁻³⁶. Some viral genomes happen to be circular RNAs, such as hepatitis D virus and plant viroids³³. In recent years, thousands of circRNAs have been identified in eukaryotes, including fungi, plants, insects, fish, and mammals via high-throughput RNA sequencing and circRNA-specific bioinformatics³⁶. Unlike linear mRNA, circRNA is highly stable as its covalently closed ring structure protects it from exonuclease-mediated degradation³⁶⁻³⁸. So far, only a few endogenous circRNAs have been shown to function as protein translation templates³⁹⁻⁴². Although circRNA lacks the essential elements for cap-dependent translation, it can be engineered to enable protein translation through internal ribosome entry site (IRES) or the m6A modification incorporated to the upstream of open reading frame (ORF)^{43,44}.

### BRIEF SUMMARY

The present application provides novel vaccines against a SARS-CoV-2 variant (e.g., Delta or Omicron) based on circular RNAs (circRNA). Also provided are methods of producing the circRNA vaccines and methods of treating or preventing a SARS-CoV-2 infection using the circRNA vaccines.

One aspect of the present application provides a circular RNA (circRNA) comprising a nucleic acid sequence encoding an antigenic polypeptide of a SARS-CoV-2 variant, wherein the SARS-CoV-2 variant is a Delta variant or an Omicron variant. In some embodiments, the antigenic polypeptide comprises a Spike(S) protein or a fragment thereof of the SARS-CoV-2 variant. In some embodiments, the antigenic polypeptide comprises a receptor-binding domain (RBD) of the S protein. In some embodiments, the RBD comprises amino acid residues 319 to 542 of a full-length S protein of SARS-CoV-2, wherein the numbering is based on SEQ ID NO: 1. In some embodiments, the SARS-CoV-2 variant is a Delta variant, and wherein the antigenic polypeptide comprises the amino acid sequence of SEQ ID NO: 18. In some embodiments, the SARS-CoV-2 variant is an Omicron variant, and wherein the antigenic polypeptide comprises the amino acid sequence of SEQ ID NO: 19.

In some embodiments according to any one of the circRNAs described above, the antigenic polypeptide further comprises a multimerization domain. In some embodiments, the multimerization domain is a C-terminal Foldon (Fd) domain of a T4 fibritin protein that mediates trimerization of the T4 fibritin protein. In some embodiments, the Fd domain comprises the amino acid sequence of SEQ ID NO: 3.

In some embodiments according to any one of the circRNAs described above, the circRNA further comprises a Kozak sequence operably linked to the nucleic acid sequence encoding the antigenic polypeptide.

In some embodiments according to any one of the circRNAs described above, the circRNA further comprises an in-frame 2A peptide coding sequence operably linked to the 3′ end of the nucleic acid sequence encoding the antigenic polypeptide.

In some embodiments according to any one of the circRNAs described above, the circRNA further comprises an internal ribosomal entry site (IRES) sequence operably linked to the nucleic acid sequence encoding the antigenic polypeptide. In some embodiments, the IRES sequence is an IRES sequence selected from the group consisting of CVB3 virus, EV71 virus, EMCV virus, PV virus, and CSFV virus IRES sequences. In some embodiments, the circRNA comprises a nucleic acid sequence comprising from the 5′ end to the 3′ end: the IRES sequence, the Kozak sequence, and the nucleic acid sequence encoding the antigenic polypeptide. In some embodiments, the circRNA further comprises a polyAC or polyA sequence disposed at the 5′ end of the IRES sequence.

In some embodiments according to any one of the circRNAs described above, the circRNA further comprises an m6A modification motif sequence operably linked to the nucleic acid sequence encoding the antigenic polypeptide. In some embodiments, the circRNA further comprises a nucleic acid sequence comprising from the 5′ end to the 3′ end: the m6A modification motif sequence, the Kozak sequence, and the nucleic acid sequence encoding the antigenic polypeptide.

In some embodiments according to any one of the circRNAs described above, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide.

Another aspect of the present application provides a composition comprising a plurality of circRNAs according to any one of the circRNAs described above, wherein the antigenic polypeptides corresponding to the plurality of circRNAs are different with respect to each other. In some embodiments, the plurality of circRNAs target a plurality of of SARS-CoV-2 variants.

One aspect of the present application provides a circRNA vaccine comprising any one of the circRNAs described above or any one of the compositions described above. In some embodiments, the circRNA vaccine further comprises a transfection agent. In some embodiments, the transfection agent is polyethylenimine (PEI). In some embodiments, the transfection agent is a lipid nanoparticle (LNP). In some embodiments, the LNP comprises an ionizable lipid, PEG2000-DMG, DSPC and cholesterol. In some embodiments, the circRNA is not formulated with a transfection agent.

Also provided is a pharmaceutical composition comprising any one of the circRNAs described above and a pharmaceutically acceptable carrier. In some embodiments, the pharmaceutical composition further comprises a transfection agent. In some embodiments, the transfection agent is PEI. In some embodiments, the transfection agent is a LNP. In some embodiments, the LNP comprises an ionizable lipid, PEG2000-DMG, DSPC and cholesterol. In some embodiments, the circRNA is not formulated with a transfection agent.

Further provided is a method of treating or preventing a SARS-CoV-2 infection in an individual, comprising administering to the individual an effective amount of any one of the circRNAs described above, any one of the compositions described above, any one of the pharmaceutical compositions described above, or any one of the circRNA vaccines described above. In some embodiments, the circRNA is administered as naked circRNA. In some embodiments, the circRNA is administered as a lipid nanoparticle formulation. In some embodiments, the circRNA is administered two or more times. In some embodiments, the interval between each administration is at least about four weeks, such as at least any of five, six, seven, or eight weeks. In some embodiments, the SARS-CoV-2 infection is caused by a SARS-CoV-2 variant. In some embodiments, the SARS-CoV-2 infection is caused by more than one SARS-CoV-2 variants. In some embodiments, the SARS-CoV-2 infection is caused by a Delta variant of SARS-CoV-2. In some embodiments, the SARS-CoV-2 infection is caused by an Omicron variant of SARS-CoV-2. In some embodiments, the SARS-CoV-2 infection is caused by one or more Omicron sub-variants (e.g. BA.1, BA.1.1, BA.2, BA.3, BA.4, BA.5). In some embodiments, the SARS-CoV-2 infection is caused by one or more sublineages of Omicron sub-variants (e.g. BA.2.75.2, BA.4.6, BF.7, BQ.1.1) In some embodiments, the circRNA is subject to rolling circle translation by a ribosome in the individual.

Other aspects of the present application provide a linear RNA capable of forming any one of the circRNAs provided herein. In some embodiments, the linear RNA can be circularized by autocatalysis of a Group I intron comprising a 5′ catalytic Group I intron fragment and a 3′ catalytic Group I intron fragment. In some embodiments, the linear RNA comprises a 3′ catalytic Group I intron fragment flanking the 5′ end of a 3′ exon sequence recognizable by a Group I intron, and a 5′ catalytic Group I intron fragment flanking the 3″ end of a 5′ exon sequence recognizable by a Group I intron. In some embodiments, the 3′ catalytic Group I intron fragment comprises the sequence of SEQ ID NO: 28, and the 5′ catalytic Group I intron fragment comprises the sequence of SEQ ID NO: 29. In some embodiments, the linear RNA further comprises a 5′ homology sequence flanking the 5′ end of the 3′ catalytic Group I intron fragment, and a 3′ homology sequence flanking the 3′ end of the 5′ catalytic Group I intron fragment. In some embodiments, the 5′ homology sequence comprises the nucleic acid sequence of SEQ ID NO: 23, and the 3′ homology sequence comprises the nucleic acid sequence of SEQ ID NO: 24.

In some embodiments, the linear RNA can be circularized by a ligase (e.g., an RNA ligase). In some embodiments, the ligase is selected from the group consisting of a T4 DNA ligase (T4 Dnl), a T4 RNA ligase 1 (T4 Rnl1) and a T4 RNA ligase 2 (T4 Rnl2). In some embodiments, the linear RNA comprises a 5′ ligation sequence at the 5′ end of the nucleic acid sequence encoding the circRNA, and a 3′ ligation sequence at the 3′ end of the nucleic acid sequence encoding the circRNA, wherein the 5′ ligation sequence and the 3′ ligation sequence can be ligated to each other via the ligase.

One aspect of the present application provides a nucleic acid construct comprising a nucleic acid sequence encoding any one of the linear RNAs described above. In some embodiments, the nucleic acid construct comprises a T7 promoter operably linked to the nucleic acid sequence encoding the linear RNA.

One aspect of the present application provides a method of producing a circRNA, comprising: (a) subjecting any one of the linear RNAs described above, wherein the linear RNA comprises a 3′ catalytic Group I intron fragment flanking the 5′ end of a 3′ exon sequence recognizable by a Group I intron, and a 5′ catalytic Group I intron fragment flanking the 3′ end of a 5′ exon sequence recognizable by a Group I intron, to a condition that activates autocatalysis of the 5′ catalytic Group I intron fragment and the 3′ catalytic Group I intron fragment to provide a circularized RNA product; and (b) isolating the circularized RNA product, thereby providing the circRNA vaccine.

One aspect of the present application provides a method of producing a circRNA, comprising: (a) contacting any one of the linear RNAs described above, wherein the linear RNA comprises a 5′ ligation sequence at the 5′ end of the nucleic acid sequence encoding the circRNA, and a 3′ ligation sequence at the 3′ end of the nucleic acid sequence encoding the circRNA, with a single-stranded adaptor nucleic acid comprising from the 5′ end to the 3″ end: a first sequence complementary to the 3′ ligation sequence and a second sequence complementary to the 5′ ligation sequence, and wherein the 5′ ligation sequence and the 3′ ligation sequence hybridize to the single-stranded adaptor nucleic acid to provide a duplex nucleic acid intermediate comprising a single strand break between the 3′ end of the 5′ ligation sequence and the 5′ end of the 3′ ligation sequence; (b) contacting the intermediate with an RNA ligase under a condition that allows ligation of the 5′ ligation sequence to the 3′ ligation sequence to provide a circularized RNA product; and (c) isolating the circularized RNA product, thereby providing the circRNA vaccine.

One aspect of the present application provides a method of producing a circRNA, comprising: (a) contacting any one of the linear RNAs described above, wherein the linear RNA comprises a 5′ ligation sequence at the 5′ end of the nucleic acid sequence encoding the circRNA, and a 3′ ligation sequence at the 3′ end of the nucleic acid sequence encoding the circRNA, with an RNA ligase under a condition that allows ligation of the 5′ ligation sequence to the 3′ ligation sequence to provide a circularized RNA product; and (b) isolating the circularized RNA product, thereby providing the circular RNA.

In some embodiments according to any one of the methods of producing a circRNA vaccine described above, the method further comprises obtaining the linear RNA by in vitro transcription of a nucleic acid construct comprising a nucleic acid sequence encoding the linear RNA. In some embodiments according to any one of the methods of producing a circRNA vaccine described above, the method further comprises purifying the circularized RNA product.

Also provided are compositions, kits and articles of manufacture for use in any one the methods described above.

### BRIEF DESCRIPTION OF THE DRAWINGS

FIGS. 1A-1I show expression of SARS-CoV-2 RBD antigens with circular RNAs in vitro. FIG. 1A shows a schematic diagram of circRNA^{RBD} circularization by the Group I ribozyme autocatalysis. SP, signal peptide sequence of human tPA protein. Foldon, the trimerization domain from bacteriophage T4 fibritin protein. RBD, the receptor binding domain of SARS-CoV-2 Spike protein. The arrows indicate the the design of primers for PCR analysis. FIG. 1B shows the agarose gel electrophoresis result of the PCR products of linear RNA^{RBD} and circRNA^{RBD}. FIG. 1C is Western Blot analysis showing the expression level of RBD antigens in the supernatant of HEK293T cells transfected with circRNA^{RBD} circularization by the Group I ribozyme autocatalysis. The circRNA^{EGFP} and linear RNA^{RBD} were set as controls. FIG. 1D shows a quantitative ELISA assay to measure the concentration of RBD antigens in the supernatant. FIG. 1E is Western Blot analysis showing the expression level of RBD antigens in the supernatant of mouse NIH3T3 cells transfected with circRNA^{RBD}. The circRNA^{EGFP} was set as controls. FIG. 1F is a schematic diagram of circRNA^{RBD} circularization by the T4 RNA ligase. SP, signal peptide sequence of human tPA protein. Foldon, the trimerization domain from bacteriophage T4 fibritin protein. RBD, the receptor binding domain of SARS-CoV-2 Spike protein. FIG. 1G is Western Blot analysis showing the expression level of RBD antigens in the supernatant of HEK293T cells transfected with circRNA^{RBD} circularization by the T4 RNA ligase. The circRNA^{EGFP} and linear RNA^{RBD} were set as controls. FIG. 1H shows a quantitative ELISA assay to measure the concentration of RBD antigens in the supernatant. FIG. 1I shows quantification of the competitive inhibition of SARS-CoV-2 pseudovirus infection (EGFP) by the circRNA^{RBD}-translated RBD antigens. The circRNA EGFP and linear RNA^{RBD} were set as controls. In FIG. 1E and FIG. 1H the data were shown as the mean±S.E.M. (n=3). In FIG. 1I, the data were shown as the mean±S.E.M. (n=2).

FIGS. 2A-2O show immunogenicity and protection of SARS-CoV-2 circRNA vaccines in mice. FIG. 2A is a schematic representation of LNP-circRNA complex. FIG. 2B shows a representative of concentration-size graph of LNP-circRNA^{RBD} measured by dynamic light scattering method. FIG. 2C is a schematic diagram of the LNP-circRNA^{RBD} vaccination process in BALB/c mice and serum collection schedule for specific antibodies analysis. FIG. 2D shows the SARS-CoV-2 specific IgG antibody titer measured with ELISA. The data were shown as the mean±S.E.M. (n=4 or 5). FIG. 2E shows a neutralization assay of VSV-based D614G pseudovirus with the serum of mice immunized with circRNA^{RBD} vaccines. The serum samples were collected at 5 weeks post boost. The data were shown as the mean±S.E.M. (n=5). FIG. 2F shows a neutralization assay of SARS-CoV-2 authentic virus with the serum of mice immunized with circRNA^{RBD} vaccines. The serum samples were collected at 5 weeks post boost. The data were shown as the mean±S.E.M. FIG. 2G shows the SARS-CoV-2 (Beta) specific IgG antibody titer of circRNA^{RBD-Beta} immunized mice measured with ELISA. The data were shown as the mean±S.E.M. (n=4 or 5). FIGS. 2H and 2I shows neutralization assays of VSV-based D614G, Alpha or Beta pseudovirus with the serum of mice immunized with circRNA^{RBD} or circRNA^{RBD-Beta} vaccines. The serum samples were collected at 1 week post boost. The data were shown as the mean±S.E.M. (n=5). FIG. 2J shows a neutralization assay of SARS-CoV-2 (Beta) authentic virus with the serum of mice immunized with circRNA^{RBD-Beta} vaccines. FIG. 2K shows a neutralization assay of SARS-CoV-2 (D614G) authentic virus with the serum of mice immunized with circRNA^{RBD-Beta} vaccines. The data were shown as the mean±S.E.M. (n=4 or 5). FIG. 2L shows an in vitro surrogate neutralization assay of circRNA^{RBD-Beta} vaccines immunized mice sera. FIG. 2M shows infected viral load of SARS-CoV-2 (Beta) authentic virus in BALB/c mice. Normalized with GAPDH. The data were shown as the mean±S.E.M. (n=3) FIG. 2N shows the weight change of immunized or placebo mice after SARS-CoV-2 (Beta) authentic virus challenge. FIG. 2O shows viral loads in the lung tissues of challenged mice. The data were shown as the mean±S.E.M. (n≥5). The NT50 was determined using infectious SARS-CoV-2 authentic virus. Each symbol represented an individual mouse. The statistical test was performed by unpaired two-sided Student's t-test.

FIGS. 3A-3O show humoral and T cell immune responses elicited by SARS-CoV-2 circRNA^{RBD-Delta} vaccines in mice. FIG. 3A shows SARS-CoV-2 (Delta) specific IgG antibody titer measured with ELISA of circRNA^{RBD-Delta} vaccines generated by Group I ribozyme autocatalysis. The data were shown as the mean±S.E.M. (n=5). FIG. 3B shows the SARS-CoV-2 (Delta) specific IgG antibody titer measured with ELISA of circRNA^{RBD-Delta} vaccines generated by T4 RNA ligase. The data were shown as the mean±S.E.M. (n=5). FIG. 3C shows a neutralization assay of VSV-based SARS-CoV-2 (Delta) pseudovirus with the serum of mice immunized with circRNA^{RBD-Delta} vaccines. The serum samples were collected at 1 week post boost. The data were shown as the mean±S.E.M. (n=5). FIGS. 3D and 3E shows neutralization assay of SARS-CoV-2 (D614G), SARS-CoV-2 (Alpha), SARS-CoV-2 (Beta) and SARS-CoV-2 (Delta) pseudovirus with the serum of placebo mice group or mice immunized with different dose circRNA^{RBD-Delta} vaccines. The serum samples were collected at 5 weeks post boost. The data were shown as the mean±S.E.M. (n=5). FIGS. 3F, 3G and 3H are FACS analysis results showing the percentages of IFN-γ, IL-2, and TNF-α positive cells evaluated among single and viable CD44⁺CD62L⁻CD8⁺ T. FIGS. 3I, 3J, 3K and 3L are FACS analysis results showing the percentages of IFN-γ, IL-2, TNF-α and IL-4 positive cells evaluated among single and viable CD44⁺CD62L⁻CD4⁺ T. In FIGS. 3F-3L, data were presented as the mean #S.E.M. (n=3 or 4). Each symbol represented an individual mouse.

FIGS. 4A-4N show circRNA vaccine-elicited higher proportion of neutralizing antibodies and stronger Th1-biased T cell immune responses than mRNA vaccine. FIG. 4A compares antigen expression level of circRNA, 1mΨ mRNA and non-modified mRNA through lipofectamine transfection in HEK293T by ELISA in different timepoint. The data were shown as the mean±S.E.M. (n=3). FIG. 4B shows dynamic change of RNA level in a. The data were shown as the mean±S.E.M. (n=3) FIG. 4C compares antigen expression level of circRNA, 1mΨ mRNA and non-modified mRNA through LNP transfection in HEK293T by ELISA in different timepoint. The data were shown as the mean±S.E.M. (n=3). FIG. 4D is Western Blot analysis showing the expression level of RBD antigens in the supernatant of HEK293T cells transfected with circRNA^{RBD}. The circRNA^{RBD} were stored at room temperature and transfection in day 0, day 3, day 7 and day 14. The untreated and linear precursor were set as controls. FIGS. 4E, 4F and 4G show quantitative ELISA to measure the expression level of LNP transfected circRNA^{RBD}, 1mΨ mRNA and non-modified mRNA. The LNP-RNA were stored at different temperature and transfection in different timepoints. The data were shown as the mean±S.E.M. (n=3). FIG. 4H shows SARS-CoV-2 (Delta) specific IgG antibody titer measured by ELISA of circRNA^{RBD}, 1mΨ mRNA immunized mice serum. The data were shown as the mean±S.E.M. (n=5). FIG. 4I shows SARS-CoV-2 (Delta) specific IgG isotype titer measured by ELISA of circRNA^{RBD-Delta}, 1mΨ mRNA^{RBD-Delta} immunized mice serum. The data were shown as the mean±S.E.M. (n=5). FIG. 4J shows SARS-CoV-2 (Delta) specific IgG2a/IgG1, IgG2c/IgG1 and (IgG2a+IgG2c)/IgG1 ratio of circRNA^{RBD-Delta} 1mΨ mRNA^{RBD-Delta} immunized mice serum. The data were shown as the mean±S.E.M. (n=5). FIGS. 4K, 4L and 4M show neutralization assays of VSV-based SARS-CoV-2 (Delta) pseudovirus with the serum of mice immunized with different dose circRNA^{RBD-Delta} or 1mΨ mRNA^{RBD-Delta} vaccines. The serum samples were collected at 1 week post boost. The data were shown as the mean±S.E.M. (n=5). FIG. 4N shows neutralizing antibodies/binding antibodies ratio of circRNA^{RBD-Delta} or 1mΨ mRNA^{RBD-Delta} vaccines. The ratio is calculated by the NT50 divide endpoint IgG titer of each mouse. The data were shown as the mean±S.E.M. (n=5).

FIGS. 5A-5J show circRNA vaccine-elicited Immunogenicity and protection against SARS-CoV-2 infection in rhesus macaques. FIG. 5A shows a schematic diagram of the LNP-circRNA^{RBD} vaccination process in rhesus macaques and serum collection schedule for specific antibodies analysis. FIG. 5B shows SARS-CoV-2 (D614G) specific IgG antibody titer of circRNA^{RBD} immunized rhesus macaques measured with ELISA. The data were shown as the mean±S.E.M. (n=4). FIG. 5C shows the NT50 of circRNA^{RBD} immunized rhesus macaques measured by VSV-based SARS-CoV-2 (D614G) pseudovirus. The data were shown as the mean±S.E.M. (n=4). FIG. 5D shows a neutralization assay of VSV-based SARS-CoV-2 (D614G), SARS-CoV-2 (Alpha), SARS-CoV-2 (Beta) and SARS-CoV-2 (Delta) pseudovirus with the serum of mice immunized with different dose circRNA^{RBD} vaccines. The data were shown as the mean±S.E.M. (n=4). FIG. 5E shows a neutralization assay of SARS-CoV-2 (D614G), SARS-CoV-2 (Alpha), SARS-CoV-2 (Beta) and SARS-CoV-2 (Delta) authentic virus with the serum of mice immunized with different dose circRNA^{RBD} vaccines. The data were shown as the mean±S.E.M. (n=4). FIG. 5F shows specific SARS-CoV-2 (D614G) IFN-γ, IL-2 and IL-4 response of PBMC from circRNA^{RBD} vaccines immunized rhesus macaques measured by ELISPOT. Each dot represents an independent biological replication. FIG. 5G shows viral loads (N gene) and subgenome RNA loads (E gene) in the lung tissues of challenged rhesus macaques. The data were shown as the mean±S.E.M. (n=4). FIG. 5H shows pathological section and HE stain of lung lobe in circRNA^{RBD} vaccines immunized rhesus macaques or control groups. FIG. 5I shows pathological score of circRNA^{RBD} vaccines immunized rhesus macaques or control groups. The data were shown as the mean±S.E.M. (n=4). FIG. 5J shows B cell (B), T cell (T) and protection ranking of each rhesus macaque in different dose groups.

FIGS. 6A-6F show circRNA vaccine caused no obvious clinical signs of illness in rhesus macaques. FIGS. 6A, 6B, 6C, 6D, and 6E show IL-6, MCP1, TNF-α, IL-1β and IFNα levels in plasma of immunized rhesus macaques. FIG. 6F shows the body temperature of rhesus macaques. Body temperature is monitored within three days after immunization.

FIG. 7 shows pseudovirus neutralization against Omicron Variant by circRNA vaccines. Neutralization assay of VSV-based SARS-CoV-2 (Omicron) pseudovirus and corresponding pseudovirus with the serum of mice immunized with 10 μg of LNP-circRNA^{RBD} or LNP-circRNA^{RBD-Beta} or LNP-circRNA^{RBD-Delta} vaccines. The serum samples were collected at 2 week post boost. The data were shown as the mean±S.E.M. (n=5 or 6).

FIGS. 8A-8B show circRNA^{RBD-Omicron} vaccine elicited SARS-CoV-2 Omicron variant-specific antibodies. FIG. 8A shows the SARS-CoV-2 Omicron variant-specific IgG antibody titer measured with ELISA using sera from mice immunized with two-dose 5 μg or 10 μg of circRNA^{RBD-Omicron} vaccine at a 1-week interval. The data were shown as the mean±S.E.M. (n=3). FIG. 8B shows the SARS-CoV-2 Omicron variant-specific IgG antibody titer measured with ELISA using sera from mice immunized with 1-dose 30 μg of circRNA^{RBD-Omicron} vaccine. The data were shown as the mean #S.E.M. (n=3). Each symbol represented an individual mouse.

FIGS. 9A-9C show agarose gel electrophoresis and HPLC purification of circRNA^{RBD}. FIG. 9A shows the agarose gel electrophoresis result of linear RNA^{RBD} and circRNA^{RBD} with different treatment. FIG. 9B shows the agarose gel electrophoresis result of circRNA^{RBD} and linear RNA^{RBD} digested by RNase R with various time from 5 min to 120 min. FIG. 9C shows HPLC chromatograms of circRNA^{RBD} without RNase R treatment (left) and circRNA^{RBD} treated by RNase R (right).

FIGS. 10A-10D show the binding and neutralizing antibodies elicited by circRNA^{RBD-Beta} vaccine at 3 days before challenge. FIG. 10A provides a diagram of the construct. FIG. 10B shows the SARS-CoV-2 specific IgG antibody titers measured with ELISA. The data were shown as the mean±S.E.M. (n=5). Each symbol represented an individual mouse. FIG. 10C shows Sigmoidal curve diagram of the inhibition rate by sera of immunized mice with surrogate virus neutralization assay. In FIGS. 10B-10D, the sera from circRNA^{RBD-Beta} (50 μg) immunized mice were collected at 3 days before challenge with authentic SARS-CoV-2 Beta strain.

FIG. 11 shows flow panel and gating strategy to quantify SARS-CoV-2-RBD-specific T cells in mice. The plots showed the gating strategy of single and viable T cells from spleens. CD4⁺ or CD8⁺ Tem cells (CD44⁺CD62⁻) were further analyzed with the expression of cytokines.

FIGS. 12A-12M show both the circRNA^{RBD-Delta} vaccine and circRNA^{RBD-Omicron} elicited high-level neutralizing antibodies against Omicron variant circRNA. FIG. 12A shows a neutralization assay of VSV-based SARS-CoV-2 pseudovirus with the serum of mice immunized with 10 μg of LNP-circRNA^{RBD}, LNP-circRNA^{RBD-Beta}, LNP-circRNA^{RBD-Delta} or LNP-1mΨ-mRNA^{RBD-Delta} vaccines. The serum samples were collected at 2-week post boost. The data were shown as the mean±S.E.M. (n=5 or 6). FIG. 12B shows a neutralization assay of VSV-based SARS-CoV-2 pseudovirus with the serum of mice immunized with 10 μg of LNP-circRNA^{RBD-Delta} or LNP-1mΨ-mRNA^{RBD-Delta} vaccines. The serum samples were collected at 7-week post boost. The data were shown as the mean±S.E.M. (n=5). FIGS. 12C and 12D show neutralizing antibodies/binding antibodies ratio of LNP-circRNA^{RBD-Delta} or LNP-1mΨ-mRNA^{RBD-Delta} a vaccines against Omicron variant. The ratio is calculated by the NT50 divide endpoint IgG titer of each mouse. The NT50 and endpoint IgG titer are measure after 2-week post boost (FIG. 12C) or 7-week post boost (FIG. 12D). The data were shown as the mean±S.E.M. (n=5). FIG. 12E shows the Omicron-Spike specific IgG antibody endopoint titer of LNP-circRNA^{RBD-Omicron}-immunized mouse serum with ELISA. The data were shown as the mean±S.E.M. (n=5). FIG. 12F shows the NT50 of LNP-circRNA^{RBD-Omicron}-immunized mouse serum with VSV-based Omicron pseudovirus. The serum samples were collected at 1 week post boost dose at a two-week interval. The data were shown as the mean±S.E.M. (n=4 or 5). FIG. 12G shows a neutralization assay of VSV-based pseudovirus with the serum of mice immunized with 5 μg or 10 μg of LNP-circRNA^{RBD-Omicron} vaccines. The serum samples were collected at 1 week post two-week interval boost. The data were shown as the mean±S.E.M. (n=4 or 5). FIG. 12H shows a schematic diagram of the LNP-circRNA vaccination process in BALB/c mice and serum collection schedule for specific antibodies analysis. FIG. 12I shows the NT50 value of LNP-circRNA^{RBD-Beta}. LNP-circRNA^{RBD-Delta} or LNP-circRNA^{RBD-Omicron}-boosted mouse serum after receiving two-dose LNP-circRNA^{RBD-Delta} with VSV-based Delta pseudovirus. The data were shown as the mean±S.E.M. (n=3). FIG. 12J shows the NT50 value of LNP-circRNA^{RBD-Beta}. LNP-circRNA^{RBD-Delta} or LNP-circRNA^{RBD-Omicron}-boosted mouse serum after receiving two-dose LNP-circRNA^{RBD-Delta} with VSV-based Omicron pseudovirus. The data were shown as the mean±S.E.M. (n=3). FIG. 12K shows a schematic diagram of the LNP-circRNA vaccination process in BALB/c mice and serum collection schedule for specific antibodies analysis. FIG. 12L shows the NT50 value of LNP-circRNA^{RBD} or LNP-circRNA^{RBD-Delta}-boosted mouse serum after receiving two-dose LNP-circRNA^{RBD} with VSV-based Delta pseudovirus. The data were shown as the mean±S.E.M. (n=2 or 3). FIG. 12M shows the NT50 value of LNP-circRNA^{RBD} or LNP-circRNA^{RBD-Delta}-boosted mouse serum after receiving two-dose LNP-circRNA^{RBD} with VSV-based Omicron pseudovirus. The data were shown as the mean±S.E.M. (n=2 or 3). Each symbol represented an individual mouse.

FIGS. 13A-13C show the circRNA^{RBD-Delta} vaccine high-level neutralizing antibodies against Omicron variant circRNA at 7 weeks post boost. FIG. 13A shows a neutralization assay of VSV-based SARS-CoV-2 pseudovirus with the serum of mice immunized with 5 μg of LNP-circRNA^{RBD-Delta} or LNP-1mΨ-mRNA^{RBD-Delta} vaccines. The serum samples were collected at 7-week post boost. The data were shown as the mean±S.E.M. (n=5). FIGS. 13B and 13C show neutralizing antibodies/binding antibodies ratio of LNP-circRNA^{RBD-Delta} or LNP-1mΨ-mRNA^{RBD-Delta} vaccines against Omicron variant. The ratio is calculated by the NT50 divide endpoint IgG titer of each mouse. The NT50 and endpoint IgG titer are measure after 2-week post boost (FIG. 13B) or 7-week post boost (FIG. 13C). The data were shown as the mean±S.E.M. (n=5).

FIGS. 14A-14C, mice imaged by IVIS imaging system. FIG. 14A. Untreated. FIG. 14B. Injected with PBS. FIG. 14C. Injected with naked circRNA^{Luc}.

FIGS. 15A-15B, immunogenicity of SARS-CoV-2 circRNA^{RBD-Beta} vaccines comprising naked circRNA^{RBD-Beta} in mice. FIG. 15A, Schematic diagram of the circRNA^{RBD-Beta} vaccination process in BALB/c mice and serum collection schedule for specific antibodies analysis. FIG. 15B, Measuring the SARS-CoV-2 specific IgG antibody titer with ELISA. The data were shown as the geometric mean±geometric S.D.

FIGS. 16A-16B, immunogenicity of SARS-CoV-2 circRNA^{RBD-Delta} vaccines comprising naked circRNA^{RBD-Delta} in mice. FIG. 16A, Schematic diagram of the circRNA^{RBD-Delta} vaccination process in BALB/c mice and serum collection schedule for specific antibodies analysis. FIG. 16B, Measuring the SARS-CoV-2 specific IgG antibody titer with ELISA. The data were shown as the geometric mean±geometric S.D.

FIGS. 17A-17C shows immunogenicity of naked RNA^{RBD-Beta}, vaccines in mice. FIG. 17A shows schematic diagram of the naked RNA^{RBD-Beta}, vaccines vaccination process in BALB/c mice and serum collection schedule for specific antibodies analysis. FIG. 17B shows results of comparing the SARS-CoV-2 RBD-Delta specific antibodies titer induced by naked circRNA vaccine or naked mRNA vaccines. FIG. 17C shows results of comparing the SARS-CoV-2 RBD-Delta specific antibodies titer induced by naked circRNA vaccine dissolved in PBS or Ringer's solution. The data were shown as the geometric mean±geometric S.D.

FIGS. 18A-18F shows optimization of circRNA vaccine encoding Omicron BA.1 RBD antigen. FIG. 18A shows results of neutralization assay of SARS-CoV-2 pseudovirus with the sera of mice immunized with circRNA^{RBD-BA.1} vaccine. FIG. 18B shows results of neutralization assay of SARS-CoV-2 pseudovirus with the sera of mice immunized with optimized circRNA^{RBD-BA.1-mut1}. FIG. 18C shows results of neutralization assay of SARS-CoV-2 pseudovirus with the sera of mice immunized with optimized circRNA^{RBD-BA.1-mut2}. FIG. 18D shows results of neutralization assay of SARS-CoV-2 pseudovirus with the sera of mice immunized with optimized circRNA^{RBD-BA.1-mut3}. FIG. 18E shows results of neutralization assay of SARS-CoV-2 pseudovirus with the sera of mice immunized with optimized circRNA^{RBD-BA.1-mut2-Folden}. FIG. 18F shows results of neutralization assay of SARS-CoV-2 pseudovirus with the sera of mice immunized with optimized circRNA^{RBD-BA.1-mut3-Folden}. The data were shown as the geometric mean±geometric S.D.

FIGS. 19A-19B shows that CircRNA^{RBD-BA.4/5} vaccine induces potent neutralizing antibodies against some variants of Omicron and its sublineage. FIG. 19A shows results of neutralization assay of SARS-CoV-2 pseudovirus with the sera of mice immunized with circRNA^{RBD-BA.4/5}, vaccine at 5 μg twice at a 3-week interval. FIG. 19B shows results of neutralization assay of SARS-CoV-2 pseudovirus with the sera of mice immunized with circRNA^{RBD-BA.4/5} vaccine at 10 μg twice at a 3-week interval. The data were shown as the geometric mean±geometric S.D.

FIGS. 20A-20D demonstrate results of neutralization assay of SARS-CoV-2 pseudovirus with the sera of mice immunized with circRNA^{RBD-BA.4/5} vaccine and show that CircRNARBD-Trimer vaccine induces potent neutralizing antibodies against some variants of Omicron and its sublineage. FIG. 20A shows results of neutralization assay of SARS-CoV-2 pseudovirus with the sera of mice immunized with 5 μg of circRNA^{RBD-Trimer 1} vaccines twice at a 2-week interval. FIG. 20B shows results of neutralization assay of SARS-CoV-2 pseudovirus with the sera of mice immunized with 10 μg of circRNA^{RBD-Trimer 1} vaccines twice at a 2-week interval. FIG. 20C shows results of neutralization assay of SARS-CoV-2 pseudovirus with the sera of mice immunized with 5 μg of circRNA^{RBD-Trimer 1-Folden} vaccines twice at a 2-week interval. FIG. 20D shows results of neutralization assay of SARS-CoV-2 pseudovirus with the sera of mice immunized with the sera of mice immunized with 10 μg of circRNA^{RBD-Trimer 1-Folden} vaccines twice at a 2-week interval. The data were shown as the geometric mean±geometric S.D.

### DETAILED DESCRIPTION

The present application provides circRNA vaccines encoding an antigenic polypeptide comprising a Spike protein or fragment thereof of a SARS-CoV-2 variant, such as a Delta or Omicron variant or sub-variants thereof. The circRNA vaccines described herein are effective against SARS-CoV-2 or its emerging variants, which induced high-proportioned neutralizing antibodies and strong Th1-biased cell responses in mice, and effectively protected mice and rhesus macaques against the infection of SARS-CoV-2 and variants.

Unlike other types of coronavirus vaccines, the circRNA vaccines described herein do not require the handling of large amounts of infectious particles during production. Furthermore, the circRNA vaccines described herein may provide enhanced stability and efficacy compared to linear RNA vaccines. For example, given their circular nature, circRNAs are particularly stable compared to many linear RNAs because they are resistant to exonucleolytic decay by the cellular exosome ribonuclease complex. In some embodiments, the circRNA in the circRNA vaccines disclosed herein can be subject to rolling circle translation by a ribosome in an individual, to whom the vaccine has been administered, giving rise to high amounts of antigenic polypeptides. The production of this circRNA vaccine could be performed using various methods, such as chemical ligation, enzyme catalysis, or ribozyme autocatalysis. The circRNA vaccines described herein provide a platform for rapid development of vaccines against emerging coronavirus strains. Moreover, circular RNAs could be quickly generated in large quantities in vitro, and they do not require any nucleotide modification, strikingly different from canonical mRNA vaccines. Our data demonstrated that an exemplary circRNA and encapsulated circRNA-LNP complex were highly thermostable at 4° C. or room temperature for 7 to 14 days. Owing to their specific properties, circRNAs hold potentials in biomedical applications.

### I. Definitions

Terms are used herein as generally used in the art, unless otherwise defined as follows.

As used herein, a “variant” virus refers to an isolate of a virus whose genome sequence differs from that of a reference virus and the difference in the genome sequence confers new phenotypic properties such as increased fitness compared to the reference virus. When referring to a viral species in the present application, such as SARS-CoV-2, it is understood that the species encompass variants as well as the reference virus that was first isolated and identified. In some embodiments, the variant virus is a variant being monitored (VBM) by the Centers for Disease Control and Prevention (“CDC”) of the United States, or by the World Health Organization (“WHO”). Variants designated as VBM include those where data indicates there is a potential or clear impact on approved or authorized medical countermeasures or that have been associated with more severe disease or increased transmission but are no longer detected, or are circulating at very low levels, in the United States. In some embodiments, the variant virus described herein is a “variant of interest”, i.e., a variant with specific genetic markers that have been associated with changes to receptor binding, reduced neutralization by antibodies generated against previous infection or vaccination, reduced efficacy of treatments, potential diagnostic impact, and/or predicted increase in transmissibility and/or disease severity. In some embodiments, the variant virus described herein is a “variant of concern”, i.e., a variant for which there is evidence of an increase in transmissibility, more severe disease (e.g., increased hospitalizations and/or deaths), significant reduction in neutralization by antibodies generated during previous infection or vaccination, reduced effectiveness of treatments or vaccines, and/or diagnostic detection failures. In some embodiments, the variant virus described herein is a “variant of high consequence”, i.e., a variant of high consequence has clear evidence that prevention measures or medical countermeasures (MCMs) have significantly reduced effectiveness relative to previously circulating variants.

The terms “polynucleotide,” “nucleic acid,” “nucleotide sequence,” and “nucleic acid sequence” are used interchangeably. They refer to a polymeric form of nucleotides of any length, either deoxyribonucleotides or ribonucleotides, or analogs thereof.

“Naked circRNA” described herein refers to circRNA that is free of a delivery vehicle (e.g., liposomes, lipid nanoparticles, or colloidal parties).

The term “vaccine” is understood as being directed to an immunoactive pharmaceutical preparation. In certain embodiments, the vaccine induces adaptive immunity when administered to a host. The vaccine preparation may further contain a pharmaceutical carrier, which may be designed for the particular mode by which the vaccine is intended to be administered.

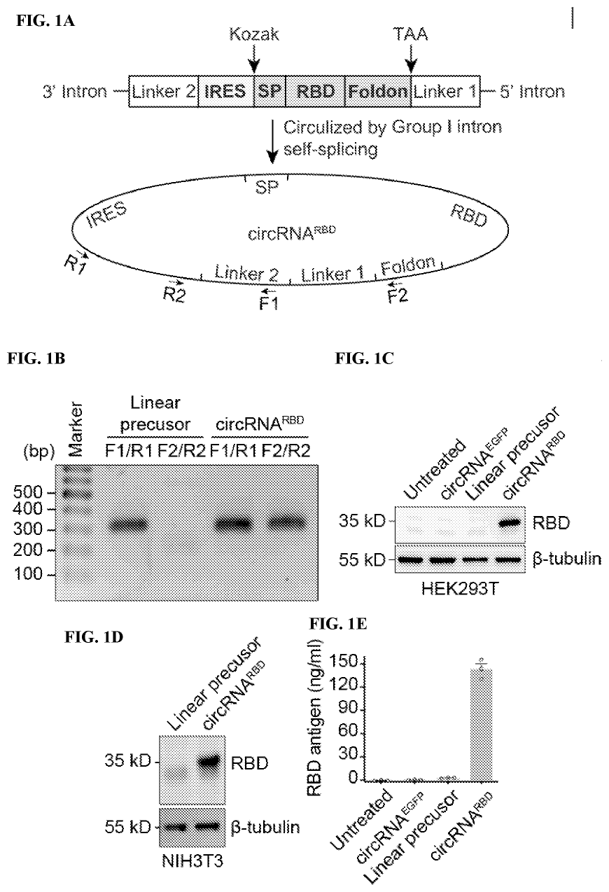

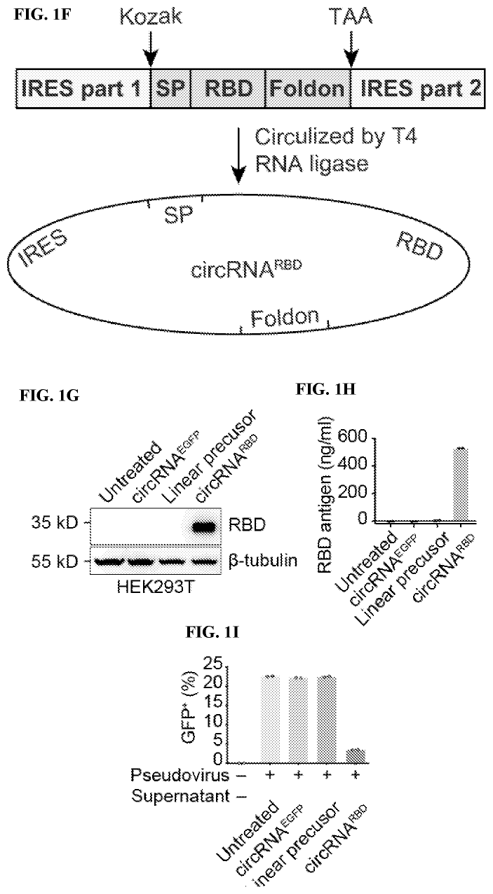

The terms “Group I intron” and “Group I catalytic intron” are used interchangeably to refer to a self-splicing ribozyme that can catalyze its own excision from an RNA precursor. Group I introns comprise two fragments, the 5′ catalytic Group I intron fragment and the 3′ catalytic Group I intron fragment, which retain their folding and catalytic function (i.e., self-splicing activity). In its native environment, the 5′ catalytic Group I intron fragment is flanked at its 5′ end by a 5′ exon, which comprises a 5′ exon sequence that is recognized by the 5′ catalytic Group I intron fragment; and the 3′ catalytic Group I intron fragment is flanked at its 3′ end by a 3′ exon, which comprises a 3′ exon sequence that is recognized by the 3′ catalytic Group I intron fragment. The terms “5′ exon sequence” and “3′ exon sequence” used herein are labeled according to the order of the exons with respect to the Group I intron in its natural environment, e.g., as shown in FIG. 1A.

The term “antigenic polypeptide” refers to a polypeptide that can be used to trigger the immune system of a mammal to develop antibodies specific to the polypeptide or a portion thereof. Antigenic polypeptides described herein include naturally-occurring proteins, protein domains, and short peptide fragments derived from a naturally-occurring protein. An antigenic polypeptide may contain one or more known epitopes of a naturally-occurring protein. The antigenic polypeptide may comprise a carrier protein or multimerization protein that improves immunogenicity.

The term “functional variant” of a reference protein refers to a variant polypeptide derived from the reference protein or a portion thereof, and the variant has substantially the same activity (e.g., binding to a target or enzymatic activity) as the reference protein. “Substantially the same activity” means an activity level that is at least about any one of 20%, 30%, 40%, 50%, 60%, 70%, 80%, 90%, or more as the activity of the reference protein.

The term “introducing” or “introduction” used herein means delivering one or more polynucleotides, such as circRNAs or one or more constructs including vectors as described herein, one or more transcripts thereof, to a host cell. The methods of the present application can employ many delivery systems, including but not limited to, viral, liposome, electroporation, microinjection and conjugation, to achieve the introduction of the circRNA or construct as described herein into a host cell. Conventional viral and non-viral based gene transfer methods can be used to introduce nucleic acids into mammalian cells or target tissues. Such methods can be used to administer nucleic acids encoding the circRNA of the present application to cells in culture, or in a host organism. Non-viral vector delivery systems include DNA plasmids, RNA (e.g. a transcript of a construct described herein), naked nucleic acid, and nucleic acid complexed with a delivery vehicle, such as a liposome. Viral vector delivery systems include DNA and RNA viruses, which have either episomal or integrated genomes for delivery to the host cell.

As used herein, “operably linked,” when referring to a first nucleic acid sequence that is operably linked with a second nucleic acid sequence, means a situation when the first nucleic acid sequence is placed in a functional relationship with the second nucleic acid sequence. For instance, a promoter is operably linked to a coding sequence if the promoter effects the transcription of the coding sequence. Likewise, the coding sequence of a signal peptide is operably linked to the coding sequence of a polypeptide if the signal peptide effects the extracellular secretion of that polypeptide. Generally, operably linked nucleic acid sequences are contiguous and, where necessary to join two protein coding regions, the open reading frames are aligned.

As used herein, “complementarity” refers to the ability of a nucleic acid to form hydrogen bond(s) with another nucleic acid by traditional Watson-Crick base-pairing. A percent complementarity indicates the percentage of residues in a nucleic acid molecule which can form hydrogen bonds (i.e., Watson-Crick base pairing) with a second nucleic acid (e.g., about 5, 6, 7, 8, 9, 10 out of 10, being about 50%, 60%, 70%, 80%, 90%, and 100% complementary respectively). “Perfectly complementary” means that all the contiguous residues of a nucleic acid sequence form hydrogen bonds with the same number of contiguous residues in a second nucleic acid sequence. “Substantially complementary” as used herein refers to a degree of complementarity that is at least about any one of 70%, 75%, 80%, 85%, 90%, 95%, 97%, 98%, 99%, or 100% over a region of about 40, 50, 60, 70, 80, 100, 150, 200, 250 or more nucleotides, or refers to two nucleic acids that hybridize under stringent conditions.

As used herein, “treatment” or “treating” is an approach for obtaining beneficial or desired results including clinical results. For purposes of this application, beneficial or desired clinical results include, but are not limited to, one or more of the following: decreasing one more symptoms resulting from the disease, diminishing the extent of the disease, stabilizing the disease (e.g., preventing or delaying the worsening of the disease), preventing or delaying the spread of the disease, preventing or delaying the occurrence or recurrence of the disease, delay or slowing the progression of the disease, ameliorating the disease state, providing a remission (whether partial or total) of the disease, decreasing the dose of one or more other medications required to treat the disease, delaying the progression of the disease, increasing the quality of life, and/or prolonging survival. Also encompassed by “treatment” is a reduction of pathological consequence of the disease. The methods of the present application contemplate any one or more of these aspects of treatment.

The terms “individual,” “subject” and “patient” are used interchangeably herein to describe a mammal, including humans. In some embodiments, the individual is human. In some embodiments, the individual is a rodent, such as a mouse. In some embodiments, the individual suffers from a genetic disease or condition. In some embodiments, the individual suffers from a SARS-CoV-2 infection. In some embodiments, the individual is at risk of contracting a SARS-CoV-2 infection. In some embodiments, the individual is in need of treatment.

As is understood in the art, an “effective amount” refers to an amount of a composition sufficient to produce a desired therapeutic outcome (e.g., stimulating the production of antibodies and improving immunity against one or more SARS-CoV-2 variants, reducing the severity or duration of, stabilizing the severity of, or eliminating one or more symptoms of a SARS-CoV-2 infection). For therapeutic use, beneficial or desired results include, e.g., decreasing one or more symptoms resulting from the disease (biochemical, histologic and/or behavioral), including its complications and intermediate pathological phenotypes presented during development of the disease, increasing the quality of life of those suffering from the disease, decreasing the dose of other medications required to treat the disease, enhancing effect of another medication, delaying the progression of the disease, and/or prolonging survival of patients. In some embodiments, an effective amount of the therapeutic agent may extend survival (including overall survival and progression free survival); result in an objective response (including a complete response or a partial response); relieve to some extent one or more signs or symptoms of the disease or condition; and/or improve the quality of life of the subject. In some embodiments, an effective amount is a prophylactically effective amount, which is an amount of a composition sufficient to prevent or reduce the severity of one or more future symptoms of a SARS-CoV-2 infection when administered to an individual who is susceptible and/or who may develop the SARS-CoV-2 infection. For prophylactic use, beneficial or desired results include, e.g., results such as eliminating or reducing the risk, lessening the severity of future disease, or delaying the onset of the disease (e.g., delaying biochemical, histologic and/or behavioral symptoms of the disease, its complications, and intermediate pathological phenotypes presenting during future development of the disease).

As used herein the term “wild type” is a term of the art understood by skilled persons and means the typical form of an organism, strain, gene or characteristic as it occurs in nature as distinguished from mutant or variant forms.

The present disclosure provides several types of compositions that are polynucleotide or polypeptide based, including variants and derivatives. These include, for example, substitutional, insertional, deletion and covalent variants and derivatives. The term “derivative” is synonymous with the term “variant” and generally refers to a molecule that has been modified and/or changed in any way relative to a reference molecule or a starting molecule.

As such, polynucleotides encoding peptides or polypeptides containing substitutions, insertions and/or additions, deletions and covalent modifications with respect to reference sequences, in particular, the polypeptide sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal residues or N-terminal residues) alternatively may be deleted depending on the use of the sequence, as for example, expression of the sequence as part of a larger sequence that is soluble, or linked to a solid support.

The term “identity.” refers to the overall relatedness between polymeric molecules, for example, between polynucleotide molecules (e.g. DNA molecules and/or RNA molecules) and/or between polypeptide molecules. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleic acid sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleic acid sequences can be determined using the algorithm of Meyers and Miller (CABIOS, 1989, 4:11-17), which has been incorporated into the ALIGN program (version 2.0) using a PAM 120 weight residue table, a gap length penalty of 12 and a gap penalty of 4. The percent identity between two nucleic acid sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

“Percent (%) amino acid sequence identity” with respect to the polypeptide sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32(5):1792-1797, 2004; Edgar, R. C., BMC Bioinformatics 5(1):113, 2004, each of which are incorporated herein by reference in their entirety for all purposes).

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “polypeptide” or “peptide” are used herein to encompass all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to one or more ingredients in a pharmaceutical formulation, other than an active ingredient, which is nontoxic to a subject. A pharmaceutically acceptable carrier includes, but is not limited to, a buffer, excipient, stabilizer, cryoprotectant, tonicity agent, preservative, and combinations thereof. Pharmaceutically acceptable carriers or excipients have preferably met the required standards of toxicological and manufacturing testing and/or are included on the Inactive Ingredient Guide prepared by the U.S. Food and Drug administration or other state/federal government or listed in the U.S. Pharmacopeia or other generally recognized pharmacopeia for use in mammals, and more particularly in humans.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (e.g., a package or container) or kit comprising at least one reagent, e.g., a medicament for treatment of a SARS-CoV-2 infection, or a probe for specifically detecting a biomarker described herein. In certain embodiments, the manufacture or kit is promoted, distributed, or sold as a unit for performing the methods described herein.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat disease of type X means the method is used to treat disease of types other than X.

The term “about X-Y” used herein has the same meaning as “about X to about Y.”

As used herein and in the appended claims, the singular forms “a,” “an,” or “the” include plural referents unless the context clearly dictates otherwise.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments; A, B, and C; A, B, or C; A or C; A or B; B or C; A and C; A and B; B and C; A (alone); B (alone); and C (alone).

### II. Circular RNA Vaccines

The present application provides circular RNAs (circRNAs) encoding an antigenic polypeptide of a SARS-CoV-2 variant such as any one of the SARS-CoV-2 variants as described in Section C.—“SARS-CoV-2 variants” below. Exemplary antigenic polypeptides are described in Section A.—“Antigenic polypeptides” below. The circRNA may comprise any one of the circRNA expression and/or circularization elements described in Section B—“Additional circRNA expression and circularization elements” below.

In some embodiments, the circRNA is stable for at least 7, 8, 9, 10, 11, 12, 13, 14, 15, 16, 17, 18, 19, 20, or 20 days when stored at 4° C. or at room temperature. In some embodiments, the circRNA is stable for at least 7 days when stored at 4° C. or at room temperature. In some embodiments, the circRNA is stable for at least 14 days when stored at 4° C. or at room temperature. In some embodiments, the circRNA is stable for at least 30 days when stored at 4° C. In some embodiments, the circRNA is less than 40% degraded after storage at room temperature for 14 days. In some embodiments, the circRNA is less than 30% degraded after storage at room temperature for 14 days. In some embodiments, the circRNA is less than 20% degraded after storage at room temperature for 14 days. In some embodiments, the circRNA is less than 10% degraded after storage at room temperature for 14 days. In some embodiments, the circRNA is less than 40% degraded after storage at room temperature for 7 days. In some embodiments, the circRNA is less than 30% degraded after storage at room temperature for 7 days. In some embodiments, the circRNA is less than 20% degraded after storage at room temperature for 7 days. In some embodiments, the circRNA is less than 10% degraded after storage at room temperature for 7 days. In some embodiments, the circRNA is less than 5% degraded after storage at room temperature for 7 days.

In some embodiments, the present application provides a circRNA comprising: (a) a nucleic acid sequence encoding an antigenic polypeptide of a SARS-CoV-2 variant, and (b) an internal ribosomal entry site (IRES) sequence, wherein the IRES sequence is operably linked to the nucleic acid sequence encoding the antigenic polypeptide. In some embodiments, the nucleic acid sequence further encodes a SP (e.g., human tPA or IgE SP) fused to the N-terminus of the antigenic polypeptide. In some embodiments, the circRNA further comprises a Kozak sequence operably linked to the nucleic acid sequence encoding the antigenic polypeptide. In some embodiments, the circRNA comprises a nucleic acid sequence comprising from the 5′ end to the 3′ end: the IRES sequence, the Kozak sequence, and the nucleic acid sequence encoding the antigenic polypeptide. In some embodiments, the circRNA further comprises a polyA or polyAC sequence disposed at the 5′ end of an IRES sequence. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide. In some embodiments, the circRNA further comprises a 5′ ligation sequence at the 5′ end of the circRNA, and a 3′ ligation sequence at the 3′ end of the circRNA, wherein the 5′ ligation sequence and the 3′ ligation sequence are ligated to each other via a ligase (e.g., T4 RNA ligase). In some embodiments, the SARS-CoV-2 infection is caused by one or more SARS-CoV-2 variants. In some embodiments, the SARS-CoV-2 variant is a Delta variant. In some embodiments, the SARS-CoV-2 variant is an Omicron variant. In some embodiments, the SARS-CoV-2 infection is caused by one or more Omicron sub-variants (e.g. BA.1, BA.1.1, BA.2, BA.3, BA.4, BA.5). In some embodiments, the SARS-CoV-2 infection is caused by one or more sublineages of Omicron sub-variants (e.g. BA.2.75.2, BA.4.6, BF.7, BQ.1.1).

In some embodiments, the present application provides a circRNA comprising: (a) a nucleic acid sequence encoding an antigenic polypeptide of a SARS-CoV-2 variant; (b) an IRES sequence, wherein the IRES sequence is operably linked to the nucleic acid sequence encoding the antigenic polypeptide; and (c) an in-frame 2A peptide coding sequence operably linked to the 3′ end of the nucleic acid sequence encoding the antigenic polypeptide. In some embodiments, the nucleic acid sequence further encodes a SP (e.g., human tPA or IgE SP) fused to the N-terminus of the antigenic polypeptide. In some embodiments, the circRNA further comprises a Kozak sequence operably linked to the nucleic acid sequence encoding the antigenic polypeptide. In some embodiments, the circRNA comprises a nucleic acid sequence comprising from the 5′ end to the 3′ end: the IRES sequence, the Kozak sequence, the nucleic acid sequence encoding the antigenic polypeptide, and the in-frame 2A peptide coding sequence. In some embodiments, the circRNA further comprises a polyA or polyAC sequence disposed at the 5′ end of an IRES sequence. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide. In some embodiments, the circRNA further comprises a 5′ ligation sequence at the 5′ end of the circRNA, and a 3′ ligation sequence at the 3′ end of the circRNA, wherein the 5′ ligation sequence and the 3′ ligation sequence are ligated to each other via a ligase (e.g., T4 RNA ligase). In some embodiments, the SARS-CoV-2 infection is caused by one or more SARS-CoV-2 variants. In some embodiments, the SARS-CoV-2 variant is a Delta variant. In some embodiments, the SARS-CoV-2 variant is an Omicron variant. In some embodiments, the SARS-CoV-2 infection is caused by one or more Omicron sub-variants (e.g. BA.1, BA.1.1, BA.2, BA.3, BA.4, BA.5). In some embodiments, the SARS-CoV-2 infection is caused by one or more sublineages of Omicron sub-variants (e.g. BA.2.75.2, BA.4.6, BF.7, BQ.1.1).

In some embodiments, the present application provides a circRNA comprising: (a) a nucleic acid sequence encoding an antigenic polypeptide of a SARS-CoV-2 variant; and (b) an m6A modification motif sequence operably linked to the nucleic acid sequence encoding the antigenic polypeptide. In some embodiments, the nucleic acid sequence further encodes a SP (e.g., human tPA or IgE SP) fused to the N-terminus of the antigenic polypeptide. In some embodiments, the circRNA further comprises a Kozak sequence operably linked to the nucleic acid sequence encoding the antigenic polypeptide. In some embodiments, the circRNA comprises a nucleic acid sequence comprising from the 5′ end to the 3′ end: the m6A modification motif sequence, the Kozak sequence, and the nucleic acid sequence encoding the antigenic polypeptide. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide. In some embodiments, the circRNA further comprises a 5′ ligation sequence at the 5′ end of the circRNA, and a 3′ ligation sequence at the 3′ end of the circRNA, wherein the 5′ ligation sequence and the 3′ ligation sequence are ligated to each other via a ligase (e.g., T4 RNA ligase). In some embodiments, the SARS-CoV-2 infection is caused by one or more SARS-CoV-2 variants. In some embodiments, the SARS-CoV-2 variant is a Delta variant. In some embodiments, the SARS-CoV-2 variant is an Omicron variant. In some embodiments, the SARS-CoV-2 infection is caused by one or more Omicron sub-variants (e.g. BA.1, BA.1.1, BA.2, BA.3, BA.4, BA.5). In some embodiments, the SARS-CoV-2 infection is caused by one or more sublineages of Omicron sub-variants (e.g. BA.2.75.2, BA.4.6, BF.7, BQ.1.1).

In some embodiments, the present application provides a circular RNA (circRNA) vaccine comprising a circRNA comprising a nucleic acid sequence encoding an antigenic polypeptide comprising an S protein or a fragment thereof of SARS-CoV-2.

In some embodiments, the present application provides a circular RNA (circRNA) vaccine comprising a circRNA comprising a nucleic acid sequence encoding an antigenic polypeptide comprising a Spike(S) protein or a fragment thereof of a SARS-CoV-2 variant. In some embodiments, the SARS-CoV-2 infection is caused by one or more SARS-CoV-2 variants. In some embodiments, the SARS-CoV-2 variant is a Delta variant. In some embodiments, the SARS-CoV-2 variant is an Omicron variant. In some embodiments, the SARS-CoV-2 infection is caused by one or more Omicron sub-variants (e.g. BA.1, BA.1.1, BA.2, BA.3, BA.4, BA.5). In some embodiments, the SARS-CoV-2 infection is caused by one or more sublineages of Omicron sub-variants (e.g. BA.2.75.2, BA.4.6, BF.7, BQ.1.1).

In some embodiments, the present application provides a circular RNA (circRNA) vaccine comprising a circRNA comprising a nucleic acid sequence encoding an antigenic polypeptide comprising: (a) an S protein or a fragment thereof of a SARS-CoV-2 variant; and (b) a multimerization domain. In some embodiments, the multimerization domain is a C-terminal Foldon (Fd) domain of a T4 fibritin protein that mediates trimerization of the T4 fibritin protein. In some embodiments, the multimerization domain is a GCN-4 based isoleucine zipper domain. In some embodiments, the multimerization domain comprises an amino acid sequence set forth in SEQ ID NOs: 3-4. In some embodiments, the multimerization domain is fused to the RBD domain of the S protein via a peptide linker, e.g., a peptide linker comprising the amino acid sequence of SEQ ID NO: 5. In some embodiments, the SARS-CoV-2 infection is caused by one or more SARS-CoV-2 variants. In some embodiments, the SARS-CoV-2 variant is a Delta variant. In some embodiments, the SARS-CoV-2 variant is an Omicron variant. In some embodiments, the SARS-CoV-2 infection is caused by one or more Omicron sub-variants (e.g. BA.1, BA.1.1, BA.2, BA.3, BA.4, BA.5). In some embodiments, the SARS-CoV-2 infection is caused by one or more sublineages of Omicron sub-variants (e.g. BA.2.75.2, BA.4.6, BF.7, BQ.1.1).

In some embodiments, the present application provides a circular RNA (circRNA) vaccine comprising a circRNA comprising a nucleic acid sequence encoding an antigenic polypeptide comprising a receptor-binding domain (RBD) of an S protein of a SARS-CoV2 variant. In some embodiments, the RBD comprises amino acid residues 319 to 542 of a full-length S protein of SARS-CoV-2, wherein the numbering is based on SEQ ID NO: 1. In some embodiments, the RBD comprises the amino acid sequence of SEQ ID NO: 2. In some embodiments, the RBD comprises the amino acid sequence of SEQ ID NO: 41. In some embodiments, the SARS-CoV-2 infection is caused by one or more SARS-CoV-2 variants. In some embodiments, the SARS-CoV-2 variant is a Delta variant. In some embodiments, the SARS-CoV-2 variant is an Omicron variant. In some embodiments, the SARS-CoV-2 infection is caused by one or more Omicron sub-variants (e.g. BA.1, BA.1.1, BA.2, BA.3, BA.4, BA.5). In some embodiments, the SARS-CoV-2 infection is caused by one or more sublineages of Omicron sub-variants (e.g. BA.2.75.2, BA.4.6, BF.7, BQ.1.1).

In some embodiments, the present application provides a circular RNA (circRNA) vaccine comprising a circRNA comprising a nucleic acid sequence encoding an antigenic polypeptide comprising: (a) a RBD of an S protein fragment of a SARS-CoV-2 variant, and (b) a multimerization domain. In some embodiments, the RBD comprises amino acid residues 319 to 542 of a full-length S protein of SARS-CoV-2, wherein the numbering is based on SEQ ID NO: 1. In some embodiments, the multimerization domain is a C-terminal Foldon (Fd) domain of a T4 fibritin protein that mediates trimerization of the T4 fibritin protein. In some embodiments, the multimerization domain is a GCN-4 based isoleucine zipper domain. In some embodiments, the multimerization domain comprises an amino acid sequence set forth in SEQ ID NOs: 3-4. In some embodiments, the multimerization domain is fused to the RBD domain of the S protein via a peptide linker, e.g., a peptide linker comprising the amino acid sequence of SEQ ID NO: 5. In some embodiments, the SARS-CoV-2 variant is a Delta variant, and the RBD comprises the amino acid sequence of SEQ ID NO: 18. In some embodiments, the SARS-CoV-2 variant is an Omicron variant, and the RBD comprises the amino acid sequence of SEQ ID NO: 19.

In some embodiments, the present application provides a circular RNA (circRNA) vaccine comprising a circRNA comprising a nucleic acid sequence encoding an antigenic polypeptide comprising an S2 region of an S protein of a SARS-CoV2 variant. In some embodiments, the S2 region comprises amino acid residues 686 to 1273 of a full-length S protein of SARS-CoV-2, wherein the numbering is based on SEQ ID NO: 1. In some embodiments, the S2 region comprises one or more mutations (e.g., K986P and V987P) that stabilize a pre-fusion conformation of the S protein. In some embodiments, the S2 region comprises the amino acid sequence of SEQ ID NO: 6 or 7. In some embodiments, the SARS-CoV-2 variant is a Delta variant. In some embodiments, the SARS-CoV-2 variant is an Omicron variant. In some embodiments, the SARS-CoV-2 infection is caused by one or more Omicron sub-variants (e.g. BA.1, BA.1.1, BA.2, BA.3, BA.4, BA.5). In some embodiments, the SARS-CoV-2 infection is caused by one or more sublineages of Omicron sub-variants (e.g. BA.2.75.2, BA.4.6, BF.7, BQ.1.1).

In some embodiments, the present application provides a circular RNA (circRNA) vaccine comprising a circRNA comprising a nucleic acid sequence encoding an antigenic polypeptide comprising amino acid residues 2-1273 of a full-length S protein of a SARS-CoV-2 variant, wherein the numbering is based on SEQ ID NO: 1. In some embodiments, the S2 region of the S protein comprises one or more mutations (e.g., K986P and V987P) that stabilize a pre-fusion conformation of the S protein. In some embodiments, the antigenic polypeptide comprises one or more mutations (e.g., deletion of amino acid residues 681-684) that inhibit cleavage of the S protein. In some embodiments, the antigenic polypeptide comprises an amino acid sequence selected from the group consisting of SEQ ID NOs: 8-10 and 40. In some embodiments, the SARS-CoV-2 variant is a Delta variant. In some embodiments, the SARS-CoV-2 variant is an Omicron variant. In some embodiments, the SARS-CoV-2 infection is caused by one or more Omicron sub-variants (e.g. BA.1, BA.1.1, BA.2, BA.3, BA.4, BA.5). In some embodiments, the SARS-CoV-2 infection is caused by one or more sublineages of Omicron sub-variants (e.g. BA.2.75.2, BA.4.6, BF.7, BQ.1.1).

In some embodiments, the present application provides a circular RNA (circRNA) vaccine comprising a circRNA comprising: (a) a nucleic acid sequence encoding an antigenic polypeptide comprising an S protein or a fragment thereof of a SARS-CoV-2 variant, and (b) an internal ribosomal entry site (IRES) sequence, wherein the IRES sequence is operably linked to the nucleic acid sequence encoding the antigenic polypeptide. In some embodiments, the nucleic acid sequence further encodes a SP (e.g., human tPA or IgE SP) fused to the N-terminus of the S protein or fragment thereof. In some embodiments, the circRNA further comprises a Kozak sequence operably linked to the nucleic acid sequence encoding the antigenic polypeptide. In some embodiments, the circRNA comprises a nucleic acid sequence comprising from the 5′ end to the 3′ end: the IRES sequence, the Kozak sequence, the SP, and the nucleic acid sequence encoding the antigenic polypeptide. In some embodiments, the circRNA further comprises a polyA or polyAC sequence disposed at the 5′ end of an IRES sequence. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide. In some embodiments, the circRNA further comprises a 5′ ligation sequence at the 5′ end of the circRNA, and a 3′ ligation sequence at the 3′ end of the circRNA, wherein the 5′ ligation sequence and the 3′ ligation sequence are ligated to each other via a ligase (e.g., T4 RNA ligase). In some embodiments, the antigenic polypeptide comprises a RBD of the S protein. In some embodiments, the antigenic polypeptide further comprises a multimerization domain (e.g., C-terminal Fd domain, or a GCN-4 based isoleucine zipper domain). In some embodiments, the antigenic polypeptide comprises an S2 region of the S protein. In some embodiments, the antigenic polypeptide comprises amino acid residues 2-1273 of a full-length S protein of a SARS-CoV-2 variant, wherein the numbering is based on SEQ ID NO: 1. In some embodiments, the S2 region of the S protein comprises one or more mutations (e.g., K986P and V987P) that stabilize a pre-fusion conformation of the S protein. In some embodiments, the antigenic polypeptide comprises one or more mutations (e.g., deletion of amino acid residues 681-684) that inhibit cleavage of the S protein. In some embodiments, the circRNA comprises a nucleic acid sequence selected from the group consisting of SEQ ID NOs: 20-21. In some embodiments, the SARS-CoV-2 variant is a Delta variant. In some embodiments, the SARS-CoV-2 variant is an Omicron variant. In some embodiments, the SARS-CoV-2 infection is caused by one or more Omicron sub-variants (e.g. BA.1, BA.1.1, BA.2, BA.3, BA.4, BA.5). In some embodiments, the SARS-CoV-2 infection is caused by one or more sublineages of Omicron sub-variants (e.g. BA.2.75.2, BA.4.6, BF.7, BQ.1.1).

In some embodiments, the present application provides a circular RNA (circRNA) vaccine comprising a circRNA comprising: (a) a nucleic acid sequence encoding an antigenic polypeptide comprising an S protein or a fragment thereof of a SARS-CoV-2 variant; (b) an IRES sequence, wherein the IRES sequence is operably linked to the nucleic acid sequence encoding the antigenic polypeptide; and (c) an in-frame 2A peptide coding sequence operably linked to the 3′ end of the nucleic acid sequence encoding the antigenic polypeptide. In some embodiments, the nucleic acid sequence further encodes a SP (e.g., human tPA or IgE SP) fused to the N-terminus of the S protein or fragment thereof. In some embodiments, the circRNA further comprises a Kozak sequence operably linked to the nucleic acid sequence encoding the antigenic polypeptide. In some embodiments, the circRNA comprises a nucleic acid sequence comprising from the 5′ end to the 3′ end: the IRES sequence, the Kozak sequence, the SP, the nucleic acid sequence encoding the antigenic polypeptide, and the in-frame 2A peptide coding sequence. In some embodiments, the circRNA further comprises a polyA or polyAC sequence disposed at the 5′ end of an IRES sequence. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide. In some embodiments, the circRNA further comprises a 5′ ligation sequence at the 5′ end of the circRNA, and a 3′ ligation sequence at the 3′ end of the circRNA, wherein the 5′ ligation sequence and the 3′ ligation sequence are ligated to each other via a ligase (e.g., T4 RNA ligase). In some embodiments, the antigenic polypeptide comprises a RBD of the S protein. In some embodiments, the antigenic polypeptide further comprises a multimerization domain (e.g., C-terminal Fd domain, or a GCN-4 based isoleucine zipper domain). In some embodiments, the antigenic polypeptide comprises an S2 region of the S protein. In some embodiments, the antigenic polypeptide comprises amino acid residues 2-1273 of a full-length S protein of SARS-CoV-2 variant, wherein the numbering is based on SEQ ID NO: 1. In some embodiments, the S2 region of the S protein comprises one or more mutations (e.g., K986P and V987P) that stabilize a pre-fusion conformation of the S protein. In some embodiments, the antigenic polypeptide comprises one or more mutations (e.g., deletion of amino acid residues 681-684) that inhibit cleavage of the S protein. In some embodiments, the circRNA comprises a nucleic acid sequence selected from the group consisting of SEQ ID NOs: 20-21. In some embodiments, the SARS-CoV-2 variant is a Delta variant. In some embodiments, the SARS-CoV-2 variant is an Omicron variant. In some embodiments, the SARS-CoV-2 infection is caused by one or more Omicron sub-variants (e.g. BA.1, BA.1.1, BA.2, BA.3, BA.4, BA.5). In some embodiments, the SARS-CoV-2 infection is caused by one or more sublineages of Omicron sub-variants (e.g. BA.2.75.2, BA.4.6, BF.7, BQ.1.1).

In some embodiments, the present application provides a circular RNA (circRNA) vaccine comprising a circRNA comprising: (a) a nucleic acid sequence encoding an antigenic polypeptide comprising an S protein or a fragment thereof of a SARS-CoV-2 variant, and (b) an m6A modification motif sequence operably linked to the nucleic acid sequence encoding the antigenic polypeptide. In some embodiments, the nucleic acid sequence further encodes a SP (e.g., human tPA or IgE SP) fused to the N-terminus of the S protein or fragment thereof. In some embodiments, the circRNA further comprises a Kozak sequence operably linked to the nucleic acid sequence encoding the antigenic polypeptide. In some embodiments, the circRNA comprises a nucleic acid sequence comprising from the 5′ end to the 3′ end: the m6A modification motif sequence, the Kozak sequence, the SP, and the nucleic acid sequence encoding the antigenic polypeptide. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide. In some embodiments, the circRNA further comprises a 5′ ligation sequence at the 5′ end of the circRNA, and a 3″ ligation sequence at the 3′ end of the circRNA, wherein the 5′ ligation sequence and the 3′ ligation sequence are ligated to each other via a ligase (e.g., T4 RNA ligase). In some embodiments, the antigenic polypeptide comprises a RBD of the S protein. In some embodiments, the antigenic polypeptide further comprises a multimerization domain (e.g., C-terminal Fd domain, or a GCN-4 based isoleucine zipper domain). In some embodiments, the antigenic polypeptide comprises an S2 region of the S protein. In some embodiments, the antigenic polypeptide comprises amino acid residues 2-1273 of a full-length S protein of a SARS-CoV-2 variant, wherein the numbering is based on SEQ ID NO: 1. In some embodiments, the S2 region of the S protein comprises one or more mutations (e.g., K986P and V987P) that stabilize a pre-fusion conformation of the S protein. In some embodiments, the antigenic polypeptide comprises one or more mutations (e.g., deletion of amino acid residues 681-684) that inhibit cleavage of the S protein. In some embodiments, the circRNA comprises a nucleic acid sequence selected from the group consisting of SEQ ID NOs: 20-21. In some embodiments, the SARS-CoV-2 variant is a Delta variant. In some embodiments, the SARS-CoV-2 variant is an Omicron variant. In some embodiments, the SARS-CoV-2 infection is caused by one or more Omicron sub-variants (e.g. BA.1, BA.1.1, BA.2, BA.3, BA.4, BA.5). In some embodiments, the SARS-CoV-2 infection is caused by one or more sublineages of Omicron sub-variants (e.g. BA.2.75.2, BA.4.6, BF.7, BQ.1.1).

The present application further provides a cocktail composition comprising a plurality of circRNAs each comprising a nucleic acid sequence encoding an antigenic polypeptide of a SARS-CoV-2 variant. In some embodiments, the plurality of circRNA encode antigenic polypeptides that are different with respect to each other, such as different mutants of an antigenic polypeptide (e.g., S protein or fragment thereof), or an antigenic polypeptide of a different SARS-CoV-2 variant.

### A. Antigenic Polypeptides

The circRNA vaccines described herein comprise circular RNAs (circRNA) encoding an antigenic polypeptide. In some embodiments, the antigenic polypeptide comprises a Spike(S) protein or a fragment thereof of a SARS-CoV-2 variant, such as any one of the S proteins or fragments thereof as described in the “Spike protein or fragment thereof” subsection below. In some embodiments, the antigenic polypeptide comprises a multimerization domain, such as a native multimerization domain of the S protein, or an exogenous multimerization domain. Suitable multimerization domains are described in the “Multimerization domain” subsection below. The S protein or fragment thereof may be fused to the multimerization domain via a peptide linker, such as any one of the peptide linkers described in the “peptide linker” subsection below.

An antigenic polypeptide comprises at least one epitope recognizable by a T cell receptor (TCR). In some embodiments, the antigenic polypeptide is a full-length protein or a fragment thereof, or an antigenic fusion protein that can trigger an immune response in a subject. In some embodiments, the antigenic polypeptide is a short peptide of no more than 100 amino acids long. The antigenic polypeptide can be a naturally derived peptide fragment from a protein antigen containing one or more epitopes, or an artificially designed peptide with one or more natural epitope sequences, wherein a peptide linker may optionally be placed in between adjacent epitope sequences. In some embodiments, the antigenic polypeptide comprises a single epitope of an antigenic protein. In some embodiments, the antigenic polypeptide comprises about any one of 1, 2, 3, 4, 5, 10 or more epitopes from a single antigenic protein. In some embodiments, the antigenic polypeptide comprises epitopes from a plurality (e.g., 2, 3, 4, 5, 10 or more) of different antigenic proteins. In some embodiments, the antigenic polypeptide comprises a Major Histocompatibility Complex (MHC) class I-restricted epitope. In some embodiments, the antigenic polypeptide comprises a MHC class II-restricted epitope. In some embodiments, the antigenic polypeptide comprises both MHC class I-restricted and MHC class II-restricted epitopes.

In some embodiments, the antigenic polypeptide is an antigenic protein or fragment thereof or a variant thereof of a self antigen, such as an antigen involved in a disease or condition. In some embodiments, the antigenic polypeptide is a tumor antigen peptide. Tumor antigen peptide sequences are known in the art and can be found at public databases, such as the Cancer Antigenic Peptide Database (van der Bruggen P et al. (2013) “Peptide database: T cell-defined tumor antigens.” Cancer Immunity. URL: caped.icp.ucl.ac.be). The coding RNA sequence in the linear RNA or circRNA described herein may encode any of the known tumor antigen peptides or combinations thereof. In some embodiments, the antigenic polypeptide comprises an epitope of a tumor associated antigen (TAA). In some embodiments, the antigenic polypeptide comprises an epitope of a tumor specific antigen. In some embodiments, the antigenic polypeptide comprises an epitope of a neoantigen, i.e., newly acquired and expressed antigens present in tumor cells of an individual.

In some embodiments, the amino acid sequences of one or more epitope peptides are predicted based on the sequence of the antigen protein (including neoantigens) using a bioinformatics tool for T cell epitope prediction. Exemplary bioinformatics tools for T cell epitope prediction are known in the art, for example, see Yang X. and Yu X. (2009) “An introduction to epitope prediction methods and software” Rev. Med. Virol. 19(2): 77-96. In some embodiments, the sequence of the antigen protein is known in the art or available in public databases. In some embodiments, the sequence of the antigen protein (including neoantigens) is determined by sequencing a sample (such as a tumor sample) of the individual being treated.

In some embodiments, the antigenic polypeptide comprises a Spike(S) protein or a fragment thereof of a SARS-CoV-2 variant. In some embodiments, the antigenic polypeptide is a full-length S protein. In some embodiments, the antigenic polypeptide is a fragment of a naturally occurring S protein.

In some embodiments, the antigenic polypeptide comprises a variant of an S protein or fragment thereof of SARS-CoV-2. Variants of the SARS-CoV-2 genome have been described. See, for example, Forster et al. (2020). Phylogenetic network analysis of SARS-CoV-2 genomes. PNAS 117 (17) 9241-9243, which is incorporated herein by reference in its entirety. In some embodiments, the antigenic polypeptide comprises a variant of an S protein or fragment thereof that confers a fitness advantage to a SARS-CoV-2 variant, such as enhanced infectivity. In some embodiments, the antigenic polypeptide comprises an S protein or fragment thereof of SARS-CoV-2 having a D614G mutation. In some embodiments, the antigenic polypeptide is capable of eliciting an immune response in an individual against different strains and variants of SARS-CoV-2. In some embodiments, the antigenic polypeptide is capable of eliciting an immune response in an individual against a specific strain or variant of SARS-CoV-2.

In some embodiments, the antigenic polypeptide comprises a receptor-binding domain (RBD) of an S protein of a SARS-CoV2 variant. In some embodiments, the RBD comprises amino acid residues 319 to 542 of a full-length S protein of SARS-CoV-2, wherein the numbering is based on SEQ ID NO: 1. In some embodiments, the RBD comprises an amino acid sequence having at least about 80% (e.g., at least about 85%, 90%, 95%, 98% or more, or 100%) sequence identity to the amino acid sequence of SEQ ID NO: 18. In some embodiments, the RBD comprises an amino acid sequence having at least about 80% (e.g., at least about 85%, 90%, 95%, 98% or more, or 100%) sequence identity to the amino acid sequence of SEQ ID NO: 19.

In some embodiments, the antigenic polypeptide comprises an S2 region of an S protein of a SARS-CoV2 variant. In some embodiments, the S2 region comprises amino acid residues 686 to 1273 of a full-length S protein of SARS-CoV-2, wherein the numbering is based on SEQ ID NO: 1. In some embodiments, the S2 region comprises an amino acid sequence having at least about 80% (e.g., at least about 85%, 90%, 95%, 98% or more, or 100%) sequence identity to the amino acid sequence of SEQ ID NO: 6. In some embodiments, the S2 region comprises one or more mutations that stabilize a pre-fusion conformation of the S protein. In some embodiments, the S2 region comprises K986P and V987P mutations. In some embodiments, the S2 region comprises an amino acid sequence having at least about 80% (e.g., at least about 85%, 90%, 95%, 98% or more, or 100%) sequence identity to the amino acid sequence of SEQ ID NO: 7.

In some embodiments, the antigenic polypeptide comprises both an RBD and an S2 region of an S protein of a SARS-CoV2 variant. In some embodiments, the antigenic polypeptide comprises an amino acid sequence having at least about 80% (e.g., at least about 85%, 90%, 95%, 98% or more, or 100%) sequence identity to the amino acid sequence of SEQ ID NO: 1.

In some embodiments, the antigenic polypeptide comprises a Spike(S) protein fragment of a SARS-CoV-2 variant and a multimerization domain, which can be operably linked to the S protein fragment. In some embodiments, the multimerization domain is a C-terminal Foldon (Fd) domain of a T4 fibritin protein that mediates trimerization of the T4 fibritin protein. In some embodiments, the multimerization domain is a GCN-4 based isoleucine zipper domain. In some embodiments, the multimerization domain comprises the amino acid sequence as set forth in SEQ ID NO: 3 or 4. In some embodiments, the multimerization domain is fused to the S protein fragment via a peptide linker. In some embodiments, the antigenic polypeptide comprises a RBD domain of an S protein fused to a multimerization domain via a peptide linker. In some embodiments, the peptide linker comprises the amino acid sequence of SEQ ID NO: 5.

In some embodiments, the antigenic polypeptide comprises a Spike(S) protein or a fragment thereof of SARS-CoV-2 fused to a multimerization domain. In some embodiments, the antigenic polypeptide comprises an S protein fragment fused to a C-terminal Foldon (Fd) domain (e.g., SEQ ID NO: 3) of a T4 fibritin protein that mediates trimerization of the T4 fibritin protein (e.g., SEQ ID NO: 4). In some embodiments, the antigenic polypeptide comprises an S protein fragment fused to a GCN-4 based isoleucine zipper domain. In some embodiments, the antigenic polypeptide comprises a receptor-binding domain (RBD) of an S protein of SARS-CoV-2 fused to a multimerization domain via a peptide linker. In some embodiments, the peptide linker comprises the amino acid sequence of SEQ ID NO: 5.

The antigenic polypeptide may comprise a signal peptide (SP). In some embodiments, the SP is fused to the N-terminus of the S protein or fragment thereof. In non-limiting examples, the signal peptide is the signal sequence and propeptide from human tissue plasminogen activator (tPA), the signal sequence from human IgE Immunoglobulin, or the signal peptide sequence of MHC I. In some embodiments, the signal peptide can facilitate secretion of the antigenic polypeptide encoded by the circRNA vaccine.

In some embodiments, the circRNA comprises an in-frame 2A peptide coding sequence operably linked to the 3′ end of the nucleic acid sequence encoding the antigenic polypeptide. In some embodiments, the circRNA does not comprise a stop codon at the 3′ end of the nucleic acid sequence encoding the antigenic polypeptide. In some embodiments, the in-frame 2A peptide coding sequence replaces the stop codon. In some embodiments, the circRNA contains no stop codon and the number of nucleotides composing the RNA is a multiple of three. In some embodiments, the circRNA having no stop codon and the number of nucleotides composing the RNA being a multiple of three allows for rolling circle translation of the circRNA. In some embodiments, the 2A peptide coding sequence allows for rolling circle translation of the circRNA. In some embodiments, the 2A peptide allows cleavage of a polypeptide generated by rolling circle translation into monomeric polypeptide sequences. In non-limiting examples, the 2A peptide coding sequence encodes a P2A or T2A peptide, such as the sequence set forth in SEQ ID NO: 30 or 31.

Also provided is a circRNA comprising a nucleic acid sequence encoding any one of the antigenic polypeptides described herein. The nucleic acid sequences encoding the antigenic polypeptides may be codon-optimized. In some embodiments, the circRNA comprises a nucleic acid sequence having at least about 80% (e.g., at least about 85%, 90%, 95%, 98% or more, or 100%) sequence identity to a nucleic acid sequence selected from the group consisting of SEQ ID NOs: 20-21.

### Spike Protein or Fragment Thereof.

The circRNA vaccines described herein comprise a circRNA comprising a nucleic acid sequence encoding an antigenic polypeptide comprising a Spike(S) protein or a fragment thereof of a SARS-CoV-2 variant.

In some embodiments, the S protein or fragment thereof comprises amino acid residues 2-1273 of a full-length S protein of SARS-CoV-2, wherein the numbering is based on SEQ ID NO: 1. In some embodiments, the S protein or fragment thereof comprises a deletion of amino acid residues 681-684. In some embodiments, the S protein or fragment thereof comprises at least one point mutation in the S2 region, for example, a K986P, V987P, F817P, A892P, A899P, or A942P mutation or combinations thereof. In some embodiments, the S protein of fragment thereof comprises at least one mutation selected from the mutations listed in Table A below, such as A222V, E406W, K417N, K417T, N439K, L452R, L452Q, L455N, L478K, E484K, Q493F, F490S, N501Y, A570D, D614G, P681H, A701V, T716I, S982A, or combinations thereof. In some embodiments, the S protein or fragment thereof comprises a N501Y point mutation. In some embodiments, the S protein or fragment thereof comprises K417N, E484K, and/or N501Y point mutations. In some embodiments, the S protein or fragment thereof comprises an E484K point mutation. In some embodiments, the S protein or fragment thereof comprises K417T, E484K, and N501Y point mutations. In some embodiments, the S Protein or fragment thereof of SARS-CoV-2 comprises K986P and V987P point mutations, either alone or in combination with a deletion of amino acid residues 681-684. In some embodiments, the S protein or fragment thereof comprises an amino acid sequence set forth in any one of SEQ ID NOs: 1-2, SEQ ID NOs: 6-10, SEQ ID NOs: 18-19 and SEQ ID NO: 41. In some embodiments, the S protein or fragment thereof comprises an amino acid sequence set forth SEQ ID NO: 18. In some embodiments, the S protein or fragment thereof comprises an amino acid sequence set forth SEQ ID NO: 19.

In some embodiments, the S protein or fragment thereof comprises an N-terminal domain (NTD) of an S protein of a SARS-CoV-2 variant.

In some embodiments, the S protein or fragment thereof comprises an amino acid sequence having about 80%, at least 85%, at least about 90%, at least about 95%, at least about 98% or more sequence identity to a wild-type S protein or a fragment thereof of a SARS-CoV-2 variant, or with any one of the sequences set forth in SEQ ID NOs: 1-2, SEQ ID NOs: 6-10, SEQ ID NOs: 18-19 and SEQ ID NO: 41.

### RBD Domain

In some embodiments, the S protein or fragment thereof comprises a receptor-binding domain (RBD) of the S protein. In some embodiments, the RBD comprises amino acid residues 319 to 542 of a full-length S protein of a SARS-CoV-2 variant, wherein the numbering is based on SEQ ID NO: 1. In some embodiments, the RBD comprises the amino acid sequence of SEQ ID NO: 18 or 19. In some embodiments, the RBD comprises a sequence having about 80%, at least 85%, at least about 90%, at least about 95%, at least about 98% or more sequence identity with the amino acid sequence of SEQ ID NO: 18. In some embodiments, the RBD comprises a sequence having about 80%, at least 85%, at least about 90%, at least about 95%, at least about 98% or more sequence identity with the amino acid sequence of SEQ ID NO: 19. In some embodiments, the RBD is linked to a multimerization domain. In some embodiments, the RBD is fused to a multimerization domain by a flexible peptide linker.

### S2 Region

In some embodiments, the S protein or fragment thereof comprises an S2 region of the S protein. In some embodiments, the S2 region comprises amino acid residues 686 to 1273 of a full-length S protein of SARS-CoV-2, wherein the numbering is based on SEQ ID NO: 1. In some embodiments, the S2 region comprises the amino acid sequence of SEQ ID NO: 6. In some embodiments, the S2 region comprises one or more mutations that stabilize a pre-fusion conformation of the S protein. In some embodiments, the S2 region comprises K986P and V987P mutations, for example, as in the sequence set forth in SEQ ID NO: 7. In some embodiments, the S2 region comprises a single point mutation, for example, a K986P, V987P, F817P, A892P, A899P or A942P mutation. In some embodiments, the S2 region comprises a combination of point mutations including K986P, V987P, F817P, A892P, A899P or A942P. In some embodiments, the S2 region comprises the wild type sequence of an S protein of a SARS-CoV-2 variant, such as the sequence of SEQ ID NO: 6, or a sequence having about 80%, at least 85%, at least about 90%, at least about 95%, at least about 98% or more sequence identity with the amino acid sequence of SEQ ID NO: 6.

### Multimerization Domain

In some embodiments, the antigenic polypeptide further comprises a multimerization domain, such as a dimerization domain, a trimerization domain, or a domain that mediates formation of higher order multimers. In some embodiments, the multimerization domain is a trimerization domain. In non-limiting examples, the multimerization domain comprises a C-terminal Foldon (Fd) domain of a T4 fibritin protein, wherein the C-terminal Foldon domain is the domain that mediates trimerization of the T4 fibritin protein, such as the amino acid sequence set forth in SEQ ID NO: 3. In another example, the multimerization domain comprises a GCN4-based isoleucine zipper (IZ) domain based on the trimerization domain of the GCN4 transcriptional activator from Saccharomyces cerevisiae, such as the amino acid sequence set forth in SEQ ID NO: 4. In some embodiments, the multimerization domain has about 80%, at least 85%, at least about 90%, at least about 95%, at least about 98% or more sequence identity with the amino acid sequence of SEQ ID NO: 3 or SEQ ID NO: 4. In some embodiments, the GCN4 IZ domain or T4 fibritin Fd domain can be modified to reduce their immunogenicity according to known techniques in the art. For example, the GCN4 IZ domain can be modified with N-linked glycosylation sites to reduce its immunogenicity (Sliepen et al. Immunosilencing a Highly Immunogenic Protein Trimerization Domain. The Journal of Biol. Chem. Vol. 290, No. 12, pp. 7436-7442). In some embodiments, the multimerization domain is fused to the N-terminus of the S protein or fragment thereof. In some embodiments, the multimerization domain is fused to the C-terminus of the S protein or fragment thereof.

### Peptide Linker

In some embodiments, the various domains in the antigenic polypeptide (e.g., the various domains of a Spike protein or fragment thereof) may be fused to each other or comprises domains (e.g., an antigenic polypeptide domain and a carrier protein or a multimerization domain) that are fused to each other via a peptide linker. In some embodiments, the antigenic polypeptide is a domain of an S protein of a SARS-CoV-2 fused to a multimerization domain via a peptide linker. Flexible peptide linkers such as glycine linkers, glycine-serine linkers, and linkers containing other amino acids are known in the art (for example, suitable peptide linkers are described by Chen et al. in Fusion Protein Linkers: Property, Design and Functionality. Adv. Drug Deli Rev. 2013 Oct. 15; 65(10): 1357-1369, which is incorporated herein by reference in its entirety). Peptide linkers can also be designed by computation methods. The peptide linker can be of any length from 1 to 10, 10 to 20, 20 to 30, 30 to 40, 40 to 50, or greater than 50 amino acids. In some embodiments, the peptide linker comprises the amino acid sequence of SEQ ID NO: 5.

B. Additional circRNA Expression and Circularization Elements

The circRNAs of the circRNA vaccines described herein comprise one or more additional expression elements that facilitate expression and/or circularization of the circRNA.

In some embodiments, the circRNA comprises a Kozak sequence operably linked to a nucleic acid sequence encoding an antigenic polypeptide comprising a Spike(S) protein or a fragment thereof of a SARS-CoV-2 variant. In some embodiments, the Kozak sequence functions as a protein translation initiation site.

In some embodiments, the circRNA comprises a nucleic acid sequence encoding an antigenic polypeptide comprising a Spike(S) protein or a fragment thereof of a SARS-CoV-2 variant, which is operably linked to an internal ribosomal entry site (IRES). In non-limiting examples, the IRES sequence can be a CVB3 virus, EV71 virus, EMCV virus, PV virus, or a CSFV virus IRES sequence. See, for example, Searching for IRES. RNA. 2006 October; 12(10): 1755-1785, which is incorporated herein by reference in its entirety. In some embodiments, the IRES sequence is a cellular IRES sequence. In some embodiments, the IRES sequence is followed by a Kozak sequence operably linked to the nucleic acid sequence encoding the antigenic polypeptide. In some embodiments, the circRNA further comprises a polyA or polyAC sequence disposed at the 5′ end of the IRES sequence.

In some embodiments, a polyA sequence or polyAC spacer is disposed at the 5′ end of an IRES. In some embodiments, the polyA or polyAC sequence is disposed between the 5′ end of the IRES and the exon-exon splice junction. The internal polyA sequence or polyAC spacer may range from 1 to 500 nucleotides in length (e.g., at least 20, 30, 40, 50, 60, 70, 80, 90, 120, 140, 160, 180, 200, 250, 300, 350, 400, 450, or 500 nucleotides). In some embodiments, the polyA sequence or polyAC sequence may range from 10-70, 20-60, or 30-60 nucleotides in length. In some embodiments, the circRNA comprises the polyAC sequence set forth in SEQ ID NO: 23 disposed at the 5′ end of the IRES sequence. In some embodiments, no polyA sequence or polyAC sequence is disposed at the 5′ end of the IRES sequence. Without being bound by any theory or hypothesis, an internal polyA sequence or a polyAC spacer added before IRES sequences can help to keep the functional second structure of IRES elements for efficient protein translation initiated by IRES. In some embodiments, the polyA sequence or polyAC spacer increases expression of the RNA construct.

In some embodiments, the circRNA comprises a nucleic acid sequence encoding an antigenic polypeptide comprising a Spike(S) protein or a fragment thereof of a SARS-CoV-2 variant, which is operably linked to an m6A (N⁶-methyladenosine) modification motif sequence. The m6A modification sequence can comprise an m6A consensus sequence. M6A consensus sequences are known in the art (for example, consensus sequences identified by Ke et al., 2017, m⁶A mRNA modifications are deposited in nascent pre-mRNA and are not required for splicing but do specify cytoplasmic turnover. Genes & Dev. 2017. 31:990-1006, which is incorporated herein by reference in its entirety) and available for download from GEO (GSE86336). In some embodiments, the m6A modification motif sequence comprises the sequence set forth in SEQ ID NO: 24. In some embodiments, the m6A modification motif sequence is followed by a Kozak sequence operably linked to the nucleic acid sequence encoding the antigenic polypeptide.

In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide. In some embodiments, the 3′ exon sequence recognizable by a 3″ catalytic Group I intron fragment comprises the nucleic acid sequence of SEQ ID NO: 25. In some embodiments, the 5′ exon sequence recognizable by a 5′ catalytic Group I intron fragment comprises the nucleic acid sequence of SEQ ID NO: 26. In some embodiments, the 3′ catalytic Group I intron fragment comprises the nucleic acid sequence of SEQ ID NO: 32, and the 5′ catalytic Group I intron fragment sequence comprises the nucleic acid sequence of SEQ ID NO: 33.

In some embodiments, the Group I catalytic intron of the T4 phage Td gene is bisected in such a way to preserve structural elements critical for ribozyme folding. Exon fragment 2 is then ligated upstream of exon fragment 1, and a nucleic acid sequence comprising a sequence encoding the antigenic polypeptide comprising a Spike(S) protein or fragment thereof of a SARS-CoV-2 variant is inserted between the exon-exon junction. In some embodiments, the sequence comprising an IRES or m6A sequence, a Kozak sequence, a signal peptide encoding sequence, an antigenic polypeptide comprising an S protein or fragment thereof of a SARS-CoV-2 variant, and a stop codon or in-frame 2A peptide sequence is inserted between the exon-exon junction.

In some embodiments, the circRNA comprises a 5′ ligation sequence at the 5′ end of the circRNA, and a 3′ ligation sequence at the 3′ end of the circRNA, wherein the 5′ ligation sequence and the 3′ ligation sequence are ligated to each other via a ligase (e.g., T4 RNA ligase).

### C. SARS-CoV-2 Variants

Exemplary SARS-CoV-2 variants and spike protein mutations associated with these variants are shown in Table A below. The circRNA vaccines and compositions described herein may be used for treating any one of the SARS-CoV-2 variants described herein or combinations thereof. The SARS-CoV-2 variants described herein are named by the World Health Organization or according to the Phylogenetic Assignment of Named Global Outbreak (PANGO) Lineages software. It is understood that the same variants may be referred to using different naming systems and algorithms in the art. SARS-CoV-2 variant classifications and definitions, as well as a list of known SARS-CoV-2 variants can be found at world wide web.cdc.gov/coronavirus/2019-ncov/variants/variant-classifications.html.

TABLE A
SARS-CoV-2 variants.

| WHO | | | S protein |
|---|---|---|---|
| label | Pango Lineage | Type | mutation(s) |
| Alpha | B.1.1.7 and | Variant being | 69del, 70del, 144del, |
| | lineages | monitored | (E484K*), (S494P*), |
| | | (VBM) | N501Y, A570D, |
| | | | D614G, P681H, |
| | | | T716I, S982A, |
| | | | D1118H (K1191N*) |
| Beta | B.1.351 and | VBM | D80A, D215G, |
| | descendent | | 241del, 242del, |
| | lineages | | 243del, K417N, |
| | | | E484K, N501Y, |
| | | | D614G, A701V |
| Gamma | P.1 and | VBM | L18F, T20N, P26S, |
| | descendent | | D138Y, R190S, |
| | lineages | | K417T, E484K, |
| | | | N501Y, D614G, |
| | | | H655Y, T1027I |
| Epsilon | B.1.427 | VBM | L452R, D614G |
| | B.1.429 | | S13I, W152C |
| Eta | B.1.525 | VBM | A67V, 69del, |
| | | | 70del, 144del, |
| | | | E484K, D614G, |
| | | | Q677H, F888L |
| Iota | B.1.526 | VBM | (LSF*), T95I, |
| | | | D253G, (S477N*), |
| | | | (E484K*), D614G, |
| | | | (A701V*) |
| Kappa | B.1.617.1 | VBM | (T95I), G142D, |
| | | | E154K, L452R, |
| | | | E484Q, D614G, |
| | | | P681R, Q1071H |
| N/A | B.1.617.3 | VBM | T19R, G142D, |
| | | | L452R, E484Q, |
| | | | D614G, P681R, |
| | | | D950N |
| Zeta | P.2 | VBM | E484K, (F565L*), |
| | | | D614G, V1176F |
| Mu | B.1.621, | VBM | D80G, 144del, |
| | B.1.621.1 | | F157S, L452R, |
| | | | D614G, (T791I*), |
| | | | (T859N*), D950H |
| Delta | B.1.617.2 and | Variant of | T19R, (V70F*), |
| | AY lineages | Concern | T95I, G142D, E156−, |
| | | (VOC) | F157−, R158G, |
| | | | (A222V*), |
| | | | (W258L*), |
| | | | (K417N*), L452R, |
| | | | T478K, D614G, |
| | | | P681R, D950N |
| Omicron | B.1.1.529 and | VOC | A67V, del69−70, |
| | BA lineages | | T95I, del142−144, |
| | | | Y145D, del211, |
| | | | L212I, ins214EPE, |
| | | | G339D, S371L, |
| | | | S373P, S375F, |
| | | | K417N, N440K, |
| | | | G446S, S477N, |
| | | | T478K, E484A, |
| | | | Q493R, G496S, |
| | | | Q498R, N501Y, |
| | | | Y505H, T547K, |
| | | | D614G, H655Y, |
| | | | N679K, P681H, |
| | | | N764K, D796Y, |
| | | | N856K, Q954H, |
| | | | N969K, L981F |

The reference SARS-CoV-2 virus is BetaCoV/WIV04/2019/(Accession ID: EPI-ISL-402124). In some embodiments, the SARS-CoV-2 variant is a variant being monitored, a variant of interest, a variant of concern, or a variant of high consequence. In some embodiments, the SARS-CoV-2 variant is selected from the group consisting of an Alpha (i.e., B.1.1.7 and Q) variant, a Beta (i.e., B.1.351) variant, a Gamma (i.e., P.1, also known as B.1.1.28.1) variant, an Epsilon (i.e., B.1.427 or B.1.429) variant, an Eta (i.e., B.1.525) variant, an Iota (i.e., B.1.526) variant, a Kappa (i.e., B.1.617.1) variant, a B. 1.617.3 variant, a Zeta (i.e., P.2) variant, a Mu (i.e., B.1.621 or B.1.621.1) variant, a Delta (i.e., B.1.617.2 or AY) variant, and an Omicron (i.e., B.1.1.529 or BA) variant. In some embodiments, the SARS-CoV-2 variant is a Delta variant, such as a B.1.617.2 variant, or an AY variant. In some embodiments, the SARS-CoV-2 infection is caused by one or more SARS-CoV-2 variants. In some embodiments, the SARS-CoV-2 variant is an Omicron variant, such as a B.1.529 variant or a BA variant. In some embodiments, the SARS-CoV-2 infection is caused by one or more Omicron sub-variants (e.g. BA.1, BA.1.1, BA.2, BA.3, BA.4, BA.5). In some embodiments, the SARS-CoV-2 infection is caused by one or more sublineages of Omicron sub-variants (e.g. BA.2.75.2, BA.4.6, BF.7, BQ.1.1). In some embodiments, the SARS-CoV-2 variant has one or more mutations (e.g., insertion, deletion, and/or substitution) in the spike protein. In some embodiments, the one or more mutations in the spike protein may affect viral fitness, such as transmissibility, virulence, and/or drug resistance (e.g., resistance to neutralizing antibodies and/or resistance to a vaccine). In some embodiments, the one or more mutations in the spike protein do not substantially alter viral fitness. In some embodiments, the SARS-CoV-2 variant does not have a mutation in the spike protein.

### III. Methods of Treatment

The circRNAs and compositions described herein may be used to treat or prevent a SARS-CoV-2 infection in an individual, including, but not limited to infections caused by a Delta or Omicron variant of SARS-CoV-2. In some embodiments, the circRNA is subject to rolling circle translation by a ribosome in the individual. In some embodiments, the circRNA is administered as naked circRNA. In some embodiments, the circRNA is administered as a lipid nanoparticle formulation. In some embodiments, the circRNA is administered two or more times. In some embodiments, the interval between each administration is at least about four weeks, such as at least any of five, six, seven, or eight weeks. In some embodiments, the circRNA is administered with an initial dose, followed by a secondary dose at about any of 4, 6, 8, or 10 weeks.

In some embodiments, there is provided a method of treating or preventing a SARS-CoV-2 infection in an individual, comprising administering to the individual an effective amount of a circRNA comprising a nucleic acid sequence encoding an antigenic polypeptide of a SARS-CoV-2 variant. In some embodiments, the antigenic polypeptide is an S protein or fragment thereof. In some embodiments, the method comprises administering an effective amount of a cocktail composition comprising a plurality of circRNA encoding different antigenic polypeptides. In some embodiments, the SARS-CoV-2 infection is caused by one or more SARS-CoV-2 variants. In some embodiments, the SARS-CoV-2 variant is a Delta variant. In some embodiments, the SARS-CoV-2 variant is an Omicron variant. In some embodiments, the SARS-CoV-2 infection is caused by one or more Omicron sub-variants (e.g. BA.1, BA.1.1, BA.2, BA.3, BA.4, BA.5). In some embodiments, the SARS-CoV-2 infection is caused by one or more sublineages of Omicron sub-variants (e.g. BA.2.75.2, BA.4.6, BF.7, BQ.1.1). In some embodiments, the circRNA is administered as naked circRNA. In some embodiments, the circRNA is administered as a lipid nanoparticle formulation. In some embodiments, the circRNA is administered two or more times. In some embodiments, the interval between each administration is at least about four weeks, such as at least any of five, six, seven, or eight weeks.

In some embodiments, the present application provides use of a circRNA vaccine comprising a circRNA comprising a nucleic acid sequence encoding an antigenic polypeptide comprising a Spike(S) protein or a fragment thereof of a SARS-CoV-2 variant for the manufacture of a vaccine for treating or preventing a SARS-CoV-2 infection in an individual. In some embodiments, the circRNA is administered as naked circRNA. In some embodiments, the circRNA is administered as a lipid nanoparticle formulation. In some embodiments, the circRNA is administered two or more times. In some embodiments, the interval between each administration is at least about four weeks, such as at least any of five, six, seven, or eight weeks.

In some embodiments, the present application provides a method of preventing or decreasing the risk of a SARS-CoV-2 infection in an individual, comprising administering to the individual an effective amount of the circRNA of any one of the embodiments described above, wherein the circRNA encodes an antigenic polypeptide of a SARS-CoV-2 variant. In some embodiments, the method comprises administering a cocktail composition comprising a plurality of circRNAs encoding different antigenic polypeptides. In some embodiments, the circRNA is subject to rolling circle translation by a ribosome in the individual. In some embodiments, the circRNA is administered as naked circRNA, or as a pharmaceutical composition comprising a transfection agent. In some embodiments, the SARS-CoV-2 infection is caused by one or more SARS-CoV-2 variants. In some embodiments, the SARS-CoV-2 variant is a Delta variant. In some embodiments, the SARS-CoV-2 variant is an Omicron variant. In some embodiments, the SARS-CoV-2 infection is caused by one or more Omicron sub-variants (e.g. BA.1, BA.1.1, BA.2, BA.3, BA.4, BA.5). In some embodiments, the SARS-CoV-2 infection is caused by one or more sublineages of Omicron sub-variants (e.g. BA.2.75.2, BA.4.6, BF.7, BQ.1.1). In some embodiments, the circRNA is administered as naked circRNA. In some embodiments, the circRNA is administered as a lipid nanoparticle formulation. In some embodiments, the circRNA is administered two or more times. In some embodiments, the interval between each administration is at least about four weeks, such as at least any of five, six, seven, or eight weeks.

In some embodiments, the present application provides methods of treating or preventing an infection caused by a plurality of SARS-CoV-2 strains in an individual, comprising administering to the individual an effective amount of the circRNA vaccine of any one of the embodiments described herein. In some embodiments, the method comprises administering to the individual an effective amount of multiple different circRNA vaccines of any one of the embodiments described herein. In some embodiments, the method comprises administering to the individual a composition comprising a plurality (e.g., two or more) circRNAs, wherein a first circRNA encodes an S protein or fragment thereof of a first strain of SARS-CoV-2, and a second circRNA encodes an S protein or fragment thereof of a second strain of SARS-CoV-2. In some embodiments, at least one of the circRNAs of the plurality encodes an S protein or fragment thereof comprising the mutations found in the variants of Table A, such as a Delta or an Omicron variant. In some embodiments, the circRNA is administered as naked circRNA. In some embodiments, the circRNA is administered as a lipid nanoparticle formulation. In some embodiments, the circRNA is administered two or more times. In some embodiments, the interval between each administration is at least about four weeks, such as at least any of five, six, seven, or eight weeks.

In some embodiments, the present application provides methods of treating or preventing a SARS-CoV-2 infection in an individual, comprising administering to the individual an effective amount of a circRNA vaccine comprising a circRNA comprising a nucleic acid sequence encoding an antigenic polypeptide comprising an S protein or a fragment thereof of a SARS-CoV-2 variant. In some embodiments, the antigenic polypeptide comprises a RBD of the S protein. In some embodiments, the antigenic polypeptide further comprises a multimerization domain (e.g., C-terminal Fd domain, or a GCN-4 based isoleucine zipper domain). In some embodiments, the antigenic polypeptide comprises an S2 region of the S protein. In some embodiments, the antigenic polypeptide comprises amino acid residues 2-1273 of a full-length S protein of SARS-CoV-2, wherein the numbering is based on SEQ ID NO: 1. In some embodiments, the S2 region of the S protein comprises one or more mutations (e.g., K986P and V987P) that stabilize a pre-fusion conformation of the S protein. In some embodiments, the antigenic polypeptide comprises one or more mutations (e.g., deletion of amino acid residues 681-684) that inhibit cleavage of the S protein. In some embodiments, the antigenic polypeptide comprises an S protein or fragment thereof of SARS-CoV-2 having a D614G mutation. In some embodiments, the circRNA comprises a nucleic acid sequence selected from the group consisting of SEQ ID NOs: 20-21. In some embodiments, the circRNA is subject to rolling circle translation by a ribosome in the individual. In some embodiments, the circRNA is administered as naked circRNA. In some embodiments, the circRNA is administered as a lipid nanoparticle formulation. In some embodiments, the circRNA is administered two or more times. In some embodiments, the interval between each administration is at least about four weeks, such as at least any of five, six, seven, or eight weeks.

In some embodiments, the present application provides methods of treating or preventing a SARS-CoV-2 infection in an individual, comprising administering to the individual an effective amount of a circRNA vaccine comprising a circRNA comprising: (a) a nucleic acid sequence encoding an antigenic polypeptide comprising an S protein or a fragment thereof of a SARS-CoV-2 variant, and (b) an IRES sequence, wherein the IRES sequence is operably linked to the nucleic acid sequence encoding the antigenic polypeptide. In some embodiments, the circRNA further comprises an in-frame 2A peptide coding sequence operably linked to the 3′ end of the nucleic acid sequence encoding the antigenic polypeptide. In some embodiments, the nucleic acid sequence further encodes a SP (e.g., human tPA or IgE SP) fused to the N-terminus of the S protein or fragment thereof. In some embodiments, the circRNA further comprises a Kozak sequence operably linked to the nucleic acid sequence encoding the antigenic polypeptide. In some embodiments, the circRNA comprises a nucleic acid sequence comprising from the 5′ end to the 3′ end: the IRES sequence, the Kozak sequence, the SP, and the nucleic acid sequence encoding the antigenic polypeptide. In some embodiments, the circRNA further comprises a polyA or polyAC sequence disposed at the 5′ end of an IRES sequence. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide. In some embodiments, the circRNA further comprises a 5′ ligation sequence at the 5′ end of the circRNA, and a 3″ ligation sequence at the 3′ end of the circRNA, wherein the 5′ ligation sequence and the 3″ ligation sequence are ligated to each other via a ligase (e.g., T4 RNA ligase). In some embodiments, the antigenic polypeptide comprises a RBD of the S protein. In some embodiments, the antigenic polypeptide further comprises a multimerization domain (e.g., C-terminal Fd domain, or a GCN-4 based isoleucine zipper domain). In some embodiments, the antigenic polypeptide comprises an S2 region of the S protein. In some embodiments, the antigenic polypeptide comprises amino acid residues 2-1273 of a full-length S protein of SARS-CoV-2, wherein the numbering is based on SEQ ID NO: 1. In some embodiments, the S2 region of the S protein comprises one or more mutations (e.g., K986P and V987P) that stabilize a pre-fusion conformation of the S protein. In some embodiments, the antigenic polypeptide comprises one or more mutations (e.g., deletion of amino acid residues 681-684) that inhibit cleavage of the S protein. In some embodiments, the antigenic polypeptide comprises an S protein or fragment thereof of SARS-CoV-2 having a D614G mutation. In some embodiments, the circRNA comprises a nucleic acid sequence of SEQ ID NO: 20 or SEQ ID NO:21. In some embodiments, the circRNA is subject to rolling circle translation by a ribosome in the individual. In some embodiments, the circRNA vaccine is administered via intramuscular (i.m) injection. In some embodiments, one or more doses of the circRNA vaccine are administered. In some embodiments, the interval between doses is about 2 weeks (e.g., 12, 13, 14, 15, or 16 days). In some embodiments, the method comprises administering a first dose of the circRNA vaccine and administering a second dose of the circRNA vaccine after 2 weeks or about 2 weeks. In some embodiments, the circRNA is administered as naked circRNA. In some embodiments, the circRNA is administered as a lipid nanoparticle formulation. In some embodiments, the circRNA is administered two or more times. In some embodiments, the interval between each administration is at least about four weeks, such as at least any of five, six, seven, or eight weeks.

In some embodiments, the present application provides methods of treating or preventing a SARS-CoV-2 infection in an individual, comprising administering to the individual an effective amount of a circRNA vaccine comprising a circRNA comprising: (a) a nucleic acid sequence encoding an antigenic polypeptide comprising an S protein or a fragment thereof of a SARS-CoV-2 variant, and (b) an m6A modification motif sequence operably linked to the nucleic acid sequence encoding the antigenic polypeptide. In some embodiments, the nucleic acid sequence further encodes a SP (e.g., human tPA or IgE SP) fused to the N-terminus of the S protein or fragment thereof. In some embodiments, the circRNA further comprises a Kozak sequence operably linked to the nucleic acid sequence encoding the antigenic polypeptide. In some embodiments, the circRNA comprises a nucleic acid sequence comprising from the 5′ end to the 3′ end: the m6A modification motif sequence, the Kozak sequence, the SP, and the nucleic acid sequence encoding the antigenic polypeptide. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide. In some embodiments, the circRNA further comprises a 5′ ligation sequence at the 5′ end of the circRNA, and a 3′ ligation sequence at the 3′ end of the circRNA, wherein the 5′ ligation sequence and the 3′ ligation sequence are ligated to each other via a ligase (e.g., T4 RNA ligase). In some embodiments, the antigenic polypeptide comprises a RBD of the S protein. In some embodiments, the antigenic polypeptide further comprises a multimerization domain (e.g., C-terminal Fd domain, or a GCN-4 based isoleucine zipper domain). In some embodiments, the antigenic polypeptide comprises an S2 region of the S protein. In some embodiments, the antigenic polypeptide comprises amino acid residues 2-1273 of a full-length S protein of SARS-CoV-2, wherein the numbering is based on SEQ ID NO: 1. In some embodiments, the S2 region of the S protein comprises one or more mutations (e.g., K986P and V987P) that stabilize a pre-fusion conformation of the S protein. In some embodiments, the antigenic polypeptide comprises one or more mutations (e.g., deletion of amino acid residues 681-684) that inhibit cleavage of the S protein. In some embodiments, the antigenic polypeptide comprises an S protein or fragment thereof of SARS-CoV-2 having a D614G mutation. In some embodiments, the circRNA comprises a nucleic acid sequence selected from the group consisting of SEQ ID NOs: 20-21. In some embodiments, the circRNA is subject to rolling circle translation by a ribosome in the individual. In some embodiments, the circRNA vaccine is administered via intramuscular (i.m) injection. In some embodiments, one or more doses of the circRNA vaccine are administered. In some embodiments, the interval between doses is about 2 weeks (e.g., 12, 13, 14, 15, or 16 days). In some embodiments, the method comprises administering a first dose of the circRNA vaccine and administering a second dose of the circRNA vaccine after 2 weeks or about 2 weeks. In some embodiments, the circRNA is administered as naked circRNA. In some embodiments, the circRNA is administered as a lipid nanoparticle formulation. In some embodiments, the circRNA is administered two or more times. In some embodiments, the interval between each administration is at least about four weeks, such as at least any of five, six, seven, or eight weeks.

### Formulation and Administration

In some embodiments, the circRNA composition for administration (e.g., circRNA vaccine or pharmaceutical composition) further comprises a transfection agent. In non-limiting examples, the transfection agent is polyethylenimine (PEI) or a lipid nanoparticle (LNP). Suitable lipid nanoparticles for administration of the circRNA have been described, for example, in Ickenstein, L. M. & Garidel, P. Lipid-based nanoparticle formulations for small molecules and RNA drugs. 890 Expert Opin Drug Deliv 16, 1205-1226, doi:10.1080/17425247.2019.1669558 (2019), U.S. Patent App. Pub. No. 20200121809, U.S. Patent App. Pub. No. 20200163878, U.S. Patent App. Pub. No. 20190022247, and International Patent App. Pub. No. WO2021/030701, the contents of which are herein incorporated by reference in their entirety. In some embodiments, the LNP are formed from a lipid mixture of MC3-lipid:DSPC:cholesterol:PEG2000-DMG. In some embodiments, the MC3-lipid:DSPC:cholesterol:PEG2000-DMG are mixed in molar ratios of 50:10:38.5:1.5.

Other examples of lipidosomes that can be used to administer the circRNA composition for administration (e.g., circRNA vaccine or pharmaceutical composition) include protamines, cationic nanoemulsions, modified dendrimer nanoparticles, protamine liposomes, cationic polymers, cationic polymer liposomes, polysaccharide particles, cationic lipid nanoparticles, cationic lipid-cholesterol nanoparticles, cationic lipid-cholesterol PEG nanoparticle, cationic lipid transfection reagents sold under the trademark LIPOFECTAMINE, nonliposomal transfection reagents sold under the trademark FUGENE, or any combination thereof can be used as the transfection agent.

In some embodiments, the liposome formulation may be influenced by, but not limited to, the selection of the cationic lipid component, the degree of cationic lipid saturation, the nature of the PEGylation, ratio of all components and biophysical parameters such as size. In some embodiments, the liposome formulation comprises a cationic lipid, a cholesterol and a PEGylated lipid. For example, a liposome formulation may comprise a cationic lipid, dipalmitoylphosphatidylcholine, cholesterol, and PEG-c-DMA. See, for example, Semple et al. Nature Biotech. 2010 28:172-176, herein incorporated by reference in its entirety. In some embodiments, liposome formulations may comprise from about 35 to about 45% cationic lipid, from about 40% to about 50% cationic lipid, from about 50% to about 60% cationic lipid and/or from about 55% to about 65% cationic lipid. In some embodiments, the ratio of lipid to RNA in liposomes may be from about 5:1 to about 20:1, from about 10:1 to about 25:1, from about 15:1 to about 30:1 and/or at least 30:1. Suitable liposome formulations have been described, for example, in WO2020237227, the contents of which are herein incorporated by reference in their entirety.

In some embodiments, the circRNA is delivered as naked RNA. In some embodiments, the circRNA is delivered by gene gun or by electroporation. In some embodiments, the circRNA vaccine is substantially free of a transfection agent (including for example a polyethylenimine (PEI), a liposome, and/or a lipid nanoparticle (LNP)). In some embodiments, the circRNA vaccine is substantially free of an adjuvant. In some embodiments, the circRNA vaccine comprises an adjuvant, which may enable the vaccine to elicit a higher immune response. In some embodiments, the adjuvant is not aluminum hydroxide. In some embodiments, the circRNA vaccine is substantially free of aluminum hydroxide. In some embodiments, the circRNA vaccine is substantially free or one or more (or all of) the following: protamines, cationic nanoemulsions, modified dendrimer nanoparticles, protamine liposomes, cationic polymers, cationic polymer liposomes, polysaccharide particles, cationic lipid nanoparticles, cationic lipid-cholesterol nanoparticles, cationic lipid-cholesterol PEG nanoparticle, cationic lipid transfection reagents, nonliposomal transfection reagents, or any combination thereof.

The circRNA vaccine for administration (e.g., circRNA vaccine or pharmaceutical composition) can be administered to a subject by systemic injection into the vasculature, systemic injection into the lymph nodes, subcutaneous injection or depots, or by local injection. In some embodiments, the circRNA is administered intranasally. In some embodiments, the circRNA is administered intramuscularly or intradermally.

In some embodiments, a circRNA vaccine herein (e.g., encoding an S protein or fragment thereof of a SARS-CoV-2 variant) is administered via intramuscular (i.m) injection. In some embodiments, one or more doses of the circRNA vaccine are administered. In some embodiments, two or more doses of the circRNA vaccine are administered. In some embodiments, the interval between doses is about 2 weeks (e.g., 12, 13, 14, 15, or 16 days) or more. In some embodiments, the method comprises administering a first dose of the circRNA vaccine and administering a second dose of the circRNA vaccine after 2 weeks or about 2 weeks. In some embodiments, the second dose of the circRNA is administered after more than two weeks, including for example after any of 3, 4, 5, 6, 7, or 8 weeks.

In some embodiments, the circRNA may be formulated in a lipid nanoparticle such as those described in International Publication No. WO2012170930, herein incorporated by reference in its entirety.

In some embodiments, the synthetic nanocarriers may be formulated for controlled and/or sustained release of the circRNA described herein. As a non-limiting example, the synthetic nanocarriers for sustained release may be formulated by methods known in the art, described herein and/or as described in International Pub No. WO2010138192 and US Pub No. 20100303850, each of which is herein incorporated by reference in their entirety.

In some embodiments, the circRNA may be formulated for controlled and/or sustained release wherein the formulation comprises at least one polymer that is a crystalline side chain (CYSC) polymer. CYSC polymers are described in U.S. Pat. No. 8,399,007, herein incorporated by reference in its entirety.

In some embodiments, the synthetic nanocarrier may be formulated for use as a vaccine. In some embodiments, the synthetic nanocarrier may encapsulate at least one circRNA, which encode at least one antigen. As a nonlimiting example, the synthetic nanocarrier may include at least one antigen and an excipient for a vaccine dosage form (see, International Pub No. WO201 1150264 and US Pub No. US201 10293723, each of which is herein incorporated by reference in their entirety). As another non-limiting example, a vaccine dosage form may include at least two synthetic nanocarriers with the same or different antigens and an excipient (see, International Pub No. WO201 1150249 and US Pub No. US201 10293701, each of which is herein incorporated by reference in their entirety). The vaccine dosage form may be selected by methods described herein, known in the art and/or described in International Pub No. WO201 1150258 and US Pub No. US20120027806, each of which is herein incorporated by reference in their entirety).

In some embodiments, the synthetic nanocarrier may comprise at least one circRNA, which encodes at least one adjuvant. As non-limiting example, the adjuvant may comprise dimethyldioctadecylammonium-bromide, dimethyldioctadecylammoniumchloride, dimethyldioctadecylammonium-phosphate or dimethyldioctadecylammoniumacetate (DDA) and an apolar fraction or part of said apolar fraction of a total lipid extract of a mycobacterium (See e.g., U.S. Pat. No. 8,241,610; herein incorporated by reference in its entirety). In another embodiment, the synthetic nanocarrier may comprise at least one circRNA and an adjuvant. As a non-limiting example, the synthetic nanocarrier comprising and adjuvant may be formulated by the methods described in International Pub No. WO2011150240 and US Pub No. US20110293700, each of which is herein incorporated by reference in its entirety.

In some embodiments, the circRNA functions as an adjuvant. As an example, RNA-sensing in the cytoplasm can trigger innate immunity, and innate immune signaling is known to contribute to adaptive immunity by diverse routes. Thus, the circRNA comprising the antigenic polypeptide or a second circRNA (e.g., a circRNA that does not encode a polypeptide) can be used as an adjuvant for boosting the adaptive immune response to the antigenic polypeptide.

In some embodiments, the circRNA composition for administration (e.g., circRNA vaccine or pharmaceutical composition) may be administered intranasally. For example, circRNA vaccines may be administered intranasally similar to the administration of live vaccines. In some embodiments, the circRNA may be administered intramuscularly or intradermally similarly to the administration of inactivated vaccines known in the art.

In some embodiments, the circRNA vaccine comprises an adjuvant, which may enable the vaccine to elicit a higher immune response. As a non-limiting example, the adjuvant could be a sub-micron oil-in-water emulsion, which can elicit a higher immune response in human pediatric populations (see, e.g., the adjuvant-containing vaccines described in US Patent Publication No. US20120027813 and U.S. Pat. No. 8,506,966, the contents of each of which are herein incorporated by reference in its entirety).

In some embodiments, the circRNA compositions of the present application may be administrated with other prophylactic or therapeutic compounds. As a non-limiting example, the prophylactic or therapeutic compound may be an adjuvant or a booster. As used herein, when referring to a prophylactic composition, such as a vaccine, the term “booster” refers to an extra administration of the prophylactic composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years.

### IV. Method of Preparation

The present application further provides nucleic acid constructs (e.g., linear RNA and vectors, etc.) for preparation of the circRNAs described herein, and methods for preparing the circRNAs, for example, by chemical ligation, enzymatic ligation, or ribozyme autocatalysis of linear RNAs. In some embodiments, the circRNA is prepared by circularizing a linear RNA in vitro.

### Linear RNA and Nucleic Acid Constructs Encoding Thereof

In some embodiments, the present application provides a linear RNA capable of forming the circRNA of any one of the embodiments described above. In some embodiments, the linear RNA can be circularized by chemical circularization methods using cyanogen bromide or a similar condensing agent. In some embodiments, the linear RNA can be circularized by autocatalysis of a Group I intron comprising a 5′ catalytic Group I intron fragment and a 3′ catalytic Group I intron fragment. In some embodiments, the linear RNA can be circularized by a ligase. In some embodiments, the linear RNA can be circularized by a T4 RNA ligase. In some embodiments, the linear RNA can be circularized by a DNA ligase. Suitable ligases include, but are not limited to a T4 DNA ligase (T4 Dnl), a T4 RNA ligase 1 (T4 Rnl1) and a T4 RNA ligase 2 (T4 Rnl2).

In some embodiments, the present application provides a linear RNA capable of forming the circRNA of any one of the embodiments described above, wherein the linear RNA can be circularized by autocatalysis of a Group I intron. In some embodiments, the Group I intron comprises a 5′ catalytic Group I intron fragment and a 3′ catalytic Group I intron fragment. In some embodiments, the linear RNA comprises a 3′ catalytic Group I intron fragment (such as the sequence set forth in SEQ ID NO: 32) flanking the 5′ end of a 3′ exon sequence recognizable by the 3′ catalytic Group I intron fragment (such as the sequence set forth in SEQ ID NO: 25), and the 5′ catalytic Group I intron fragment (such as the sequence set forth in SEQ ID NO: 33) flanking the 3′ end of a 5′ exon sequence recognizable by the 5′ catalytic Group I intron fragment (such as the sequence set forth in SEQ ID NO: 26).

In some embodiments, the linear RNA comprises, from 5′ to 3′ end, a 3′ Intron-IRES-Kozak-SP-Spike-5′ Intron sequence. In some embodiments, the Spike sequence comprises one of the sequences set forth in SEQ ID NOs: 11-15, 18-19 and 41.

In some embodiments, the linear RNA comprises, from 5′ to 3′ end, a 3′ Intron-IRES-Kozak-SP-RBD-5′ Intron sequence. In some embodiments, the RBD sequence comprises amino acid residues 319 to 542 of a full-length S protein of SARS-CoV-2, wherein the numbering is based on SEQ ID NO: 1.

In some embodiments, the linear RNA further comprises a 5′ homology sequence flanking the 5′ end of the 3′ catalytic Group I intron fragment, and a 3′ homology sequence flanking the 3′ end of the 5′ catalytic Group I intron fragment. In some embodiments, the linear RNA comprises, from 5′ to 3′ end, a 5′ homology arm-3 catalytic Group I Intron fragment-3′ exon sequence-IRES-Kozak-SP-antigenic polypeptide (e.g., Spike protein or fragment thereof)-5′ exon sequence-5′ catalytic Group I Intron fragment-3 homology arm sequence. In some embodiments, the homology sequence can be between 1 and 100, between 5 and 80, between 5 and 60, between 10 and 50, or between 12 and 50 nucleotides in length. In some embodiments, the homology sequence is about 20-30 nucleotides in length. In some embodiments, the 5′ homology sequence comprises the nucleic acid sequence of SEQ ID NO: 27, and the 3′ homology sequence comprises the nucleic acid sequence of SEQ ID NO: 28. In some embodiments, the homology arms increase the efficiency of RNA circularization by about 0 to 20%, more than 20%, more than 30%, more than 40%, or more than 50%.

In some embodiments, there is provided a nucleic acid construct comprising a nucleic acid sequence encoding the linear RNA. In some embodiments, a T7 promoter is operably linked to the nucleic acid sequence encoding the linear RNA. In some embodiments, the T7 promoter comprises the sequence set forth in SEQ ID NO: 29. In some embodiments, the T7 promoter is capable of driving in vitro transcription.

### Plasmids

In some embodiments, the present application provides plasmids comprising the nucleotide sequences described herein. In some embodiments, the plasmids are obtained by cloning the sequence encoding the linearized RNAs into a plasmid vector. Plasmids can be generated by techniques known in the art, such as Gibson cloning or cloning using restriction enzymes. In some embodiments, the plasmid vector includes an antibiotic expression cassette allowing antibiotic selection of bacteria expressing the plasmid. In some embodiments, the plasmids provided can be purified from bacteria and used for production of the linear circRNA constructs. Any plasmid vector suitable for in vitro transcription of the linear RNA may be used.

In some embodiments, the plasmids are linearized prior to in vitro transcription of the linear RNA. In some embodiments, the recombinant plasmids are linearized by restriction enzyme digestion. In some embodiments, the recombinant plasmids are linearized by PCR amplification. In some embodiments, the method further comprises performing in vitro transcription with the linearized plasmid template. In some embodiments, the in vitro transcription is driven by a T7 promoter.

### Linear RNA Circularized by Chemical Ligation

In some embodiments, there is provided a method of preparing a circRNA described herein, comprising: (a) chemically ligating the 5′ end and the 3′ end of a linear RNA comprising a nucleic acid sequence encoding the circRNA; and (b) isolating the circularized RNA product, thereby providing the circRNA.

In some embodiments, the step of circularizing the linear RNA comprises chemical circularization methods using cyanogen bromide or a similar condensing agent.

In some embodiments, the linear RNA can be circularized by chemical methods. In some chemical methods, the 5′-end and the 3′-end of the nucleic acid (e.g., a linear circular polyribonucleotide) includes chemically reactive groups that, when close together, may form a new covalent linkage between the 5′-end and the 3′-end of the molecule. The 5′-end may contain an NHS ester reactive group and the 3′-end may contain a 3-amino terminated nucleotide such that in an organic solvent the 3′-amino-terminated nucleotide on the 3′-end of a linear RNA molecule will undergo a nucleophilic attack on the 5′-NHS-ester moiety forming a new 5′-/3′-amide bond.

In some embodiments, the circularization efficiency of the circularization methods provided herein is at least about 10%, at least about 15%, at least about 20%, at least about 25%, at least about 30%, at least about 35%, at least about 40%, at least about 45%, at least about 50%, at least about 60, at least about 70%, at least about 80%, at least about 90%, at least about 95%, or 100%. In some embodiments, the circularization efficiency of the circularization methods provided herein is at least about 40%.

### Linear RNA Circularized by Ribozyme Autocatalysis

In some embodiments, the circRNA can be obtained by circularizing a linear RNA by ribozyme autocatalysis. In some embodiments, the linear RNA is circularized in vitro. In some embodiments, circularization by ribozyme autocatalysis comprises (a) subjecting the linear RNA to a condition that activates autocatalysis of the Group I intron (or 5′ and 3′ catalytic Group I intron fragments thereof) to provide a circularized RNA product; and (b) isolating the circularized RNA product, thereby providing the circRNA.

In some embodiments, the method comprises a step of obtaining the linear RNA by first cloning the sequence encoding the linearized RNAs into a plasmid vector, and then linearizing the recombinant plasmids. In some embodiments, the recombinant plasmids are linearized by restriction enzyme digestion. In some embodiments, the recombinant plasmids are linearized by PCR amplification. In some embodiments, the method further comprises performing in vitro transcription with the linearized plasmid template. In some embodiments, the in vitro transcription is driven by a T7 promoter. In some embodiments, the method further comprises purifying the linear RNA transcripts. In some embodiments, the linear RNAs are purified by gel purification.

In some embodiments, the present application provides a method of cyclizing a linear RNA (e.g., purified linear RNA) by ribozyme autocatalysis of the Group I intron. During splicing, the 3′ hydroxyl group of a guanosine nucleotide engages in a transesterification reaction at the 5′ splice site. The 5′ intron half is excised, and the freed hydroxyl group at the end of the intermediate engages in a second transesterification at the 3′ splice site, resulting in circularization of the intervening region and excision of the 3′ intron. In some embodiments, the condition that activates autocatalysis of the Group I intron or 5′ and 3′ catalytic Group I intron fragments is the addition of GTPs and Mg²⁺. In some embodiments, there is provided a step of cyclizing the linear RNAs by adding GTPs and Mg²⁺ at 55° C. for 15 min. In some embodiments, the method further comprises treating with RNase R to digest the linear RNA transcripts. In some embodiments, the method further comprises isolating the circular RNA (circRNA). In some embodiments, the step of isolating the circRNA comprises gel-purifying the circRNA. In some embodiments, the purified circRNA can be stored at −80° C.

In some embodiments, the circularization has an efficiency of at least 2%, at least 5%, at least 10%, at least 15%, at least 20%, at least 25%, at least 30%, at least 32%, at least 34%, at least 36%, at least 38%, at least 40%, at least 42%, at least 44%, at least 46%, at least 48%, or at least 50%. In some embodiments, the circularization has an efficiency of about 40% to about 50% or more than 50%.

### Linear RNA Circularized by Ligation

In some embodiments, the circRNA can be obtained by circularizing a linear RNA using a ligase such as a RNA ligase. In some embodiments, the linear RNA is circularized in vitro. In some embodiments, the linear RNA can be circularized by a T4 RNA ligase. In some embodiments, the linear RNA comprises a 5′ ligation sequence at the 5′ end of the nucleic acid sequence encoding the circRNA, and a 3′ ligation sequence at the 3′ end of the nucleic acid sequence encoding the circRNA, wherein the 5′ ligation sequence and the 3′ ligation sequence can be ligated to each other via the RNA ligase. In non-limiting examples, the linear RNA can be circularized by a ligase such as a T4 DNA ligase (T4 Dnl), T4 RNA ligase 1 (T4 Rnl1), and T4 RNA ligase 2 (T4 Rnl2). The linear RNA may be circularized with or without the presence of a single stranded nucleic acid adaptor, e.g., a splint DNA.

In some embodiments, the present application provides a method of producing any one of the circRNAs described above, comprising: (a) contacting any one of the linear RNAs comprising a 5′ ligation sequence at the 5′ end of the nucleic acid sequence encoding the circRNA, and a 3′ ligation sequence at the 3′ end of the nucleic acid sequence encoding the circRNA described above with a single-stranded adaptor nucleic acid comprising from the 5′ end to the 3′ end: a first sequence complementary to the 3′ ligation sequence and a second sequence complementary to the 5′ ligation sequence, and wherein the 5′ ligation sequence and the 3′ ligation sequence hybridize to the single-stranded adaptor nucleic acid to provide a duplex nucleic acid intermediate comprising a single strand break between the 3′ end of the 5′ ligation sequence and the 5′ end of the 3′ ligation sequence; (b) contacting the intermediate with an RNA ligase under a condition that allows ligation of the 5′ ligation sequence to the 3′ ligation sequence to provide a circularized RNA product; and (c) isolating the circularized RNA product, thereby providing the circRNA.

In some embodiments, the method described herein comprises circularizing a linear RNA in vitro, comprising: (a) contacting any one of the linear RNAs comprising a 5′ ligation sequence at the 5′ end of the nucleic acid sequence encoding the circular RNA, and a 3′ ligation sequence at the 3′ end of the nucleic acid sequence encoding the circular RNA described above with an RNA ligase under a condition that allows ligation of the 5′ ligation sequence to the 3′ ligation sequence to provide a circularized RNA product; and (b) isolating the circularized RNA product, thereby providing the circular RNA.

In some embodiments, the method further comprises treating with RNase R to digest the linear RNA transcripts. In some embodiments, the method further comprises isolating the circular RNA (circRNA). In some embodiments, the step of isolating the circRNA comprises gel-purifying the circRNA. In some embodiments, the purified circRNA can be stored at −80° C.

In some embodiments, a DNA or RNA ligase may be used to enzymatically link a 5′-phosphorylated nucleic acid molecule (e.g., a linear RNA) to the 3′-hydroxyl group of a nucleic acid (e.g., a linear nucleic acid) forming a new phosphorodiester linkage. In an example reaction, a linear circular RNA is incubated at 37° C. for 1 hour with 1-10 units of T4 RNA ligase (New England Biolabs, Ipswich, Mass.) according to the manufacturer's protocol. The ligation reaction may occur in the presence of a linear nucleic acid capable of base-pairing with both the 5′- and 3′-region in juxtaposition to assist the enzymatic ligation reaction. In some embodiments, the ligation is splint ligation. For example, a splint ligase, like SPLINTR® ligase, can be used for splint ligation. For splint ligation, a single stranded polynucleotide (splint), like a single stranded RNA, can be designed to hybridize with both termini of a linear polyribonucleotide, so that the two termini can be juxtaposed upon hybridization with the single-stranded splint. Splint ligase can thus catalyze the ligation of the juxtaposed two termini of the linear polyribonucleotide, generating a circular polyribonucleotide.

In some embodiments, a DNA or RNA ligase may be used in the synthesis of the circular RNA. As a non-limiting example, the ligase may be a circ ligase or circular ligase.

Purification of circRNA

In some embodiments, the method provided herein of producing a circRNA further comprises a step of purifying the circularized RNA product. In non-limiting examples, the circRNA is purified by gel-purification or by high-performance liquid chromatography (HPLC). In some embodiments, agarose gel electrophoresis allows for simple and effective separation of circular splicing products from linear precursor molecules, nicked circles, splicing intermediates, and excised introns. In some embodiments, the method comprises purifying the circular RNA by chromatography, such as HPLC. In some embodiments, the purified circular RNA can be stored at −80° C.

### V. Pharmaceutical Compositions, Kits and Articles of Manufacture

Further provided by the present application are pharmaceutical compositions comprising any one of circRNAs described herein, and a pharmaceutically acceptable carrier. Pharmaceutical compositions can be prepared by mixing the therapeutic agents described herein having the desired degree of purity with optional pharmaceutically acceptable carriers, excipients or stabilizers (Remington's Pharmaceutical Sciences 16th edition, Osol, A. Ed. (1980), which is incorporated herein by reference in its entirety), in the form of lyophilized formulations or aqueous solutions. Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers (e.g. sodium chloride), stabilizers, metal complexes (e.g. Zn-protein complexes); chelating agents such as EDTA and/or non-ionic surfactants.

In some embodiments, the pharmaceutical composition is contained in a single-use vial, such as a single-use sealed vial. In some embodiments, the pharmaceutical composition is contained in a multi-use vial. In some embodiments, the pharmaceutical composition is contained in bulk in a container. In some embodiments, the pharmaceutical composition is cryopreserved.

The present application further provides kits and articles of manufacture for use in any embodiment of the treatment methods described herein. The kits and articles of manufacture may comprise any one of the formulations and pharmaceutical compositions described herein.

In some embodiments, there is provided a kit comprising any one of the circRNAs described herein and instructions for treating or preventing a SARS-CoV-2 infection.

In some embodiments, there is provided a kit comprising any one of the plasmids or linear RNAs described herein, and instructions for preparing any one of the circRNAs. In some embodiments, there is provided a kit comprising any one of the plasmids, linear RNAs, or circRNAs described herein, and instructions for administering the circRNA.

The kits of the invention are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the compositions generally include information as to dosage, dosing schedule, and route of administration for the intended treatment. The containers may be unit doses, bulk packages (e.g., multi-dose packages) or sub-unit doses. For example, kits may be provided that contain sufficient dosages of the circRNA as disclosed herein to provide effective treatment of an individual or of many individuals. Additionally, kits may be provided that contain sufficient dosages of the circRNA to allow for multiple administrations to an individual (e.g., initial vaccine administration and subsequent booster administration, in the case of a circRNA vaccine). Kits may also include multiple unit doses of the pharmaceutical compositions and instructions for use and packaged in quantities sufficient for storage and use in pharmacies, for example, hospital pharmacies and compounding pharmacies.

In some embodiments, the kit comprises a delivery system. The delivery system may be a unit dose delivery system. The volume of solution or suspension delivered per dose can be anywhere from about 5 to about 2000 microliters, from about 10 to about 1000 microliters, or from about 50 to about 500 microliters. Delivery systems for these various dosage forms can be syringes, dropper bottles, plastic squeeze units, atomizers, nebulizers or pharmaceutical aerosols in either unit dose or multiple dose packages. In some embodiments, there is provided a delivery system of any one of the circRNAs described herein, comprising the circRNA and a device for delivering the circRNA.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended embodiments.

### Example 1. circRNA^{RBD} Produced Functional SARS-CoV-2 RBD Antigens

The Group I ribozyme autocatalysis strategy⁴³ was employed to produce circular RNAs encoding SARS-CoV-2 RBD antigens²³, termed circRNA^{RBD} (FIG. 1A). In this construct, the IRES element was placed before the RBD-coding sequence to initiate its translation. To enhance the immunogenicity of RBD antigens, the signal peptide sequence of human tissue plasminogen activator (tPA) was fused to the N-terminus of RBD to ensure the secretion of antigens^{17, 15-47}. Besides, recent research reported that RBD trimmers were superior to the monomeric RBD in binding hACE2^{6,7,48}. So the trimerization motif of bacteriophage T4 fibritin protein (foldon)⁴⁹ was fused to its C terminus, to further enhance the immunogenicity of RBD antigens. This IRES—SP-RBD-Foldon sequence was then cloned into the vector to construct the template of in vitro transcription (IVT) for producing circRNA^{RBD} (FIG. 1A). The precise circularization of circRNA^{RBD} was verified by reverse transcription-PCR analysis using specific primers and sanger sequencing (FIGS. 1A-1B).

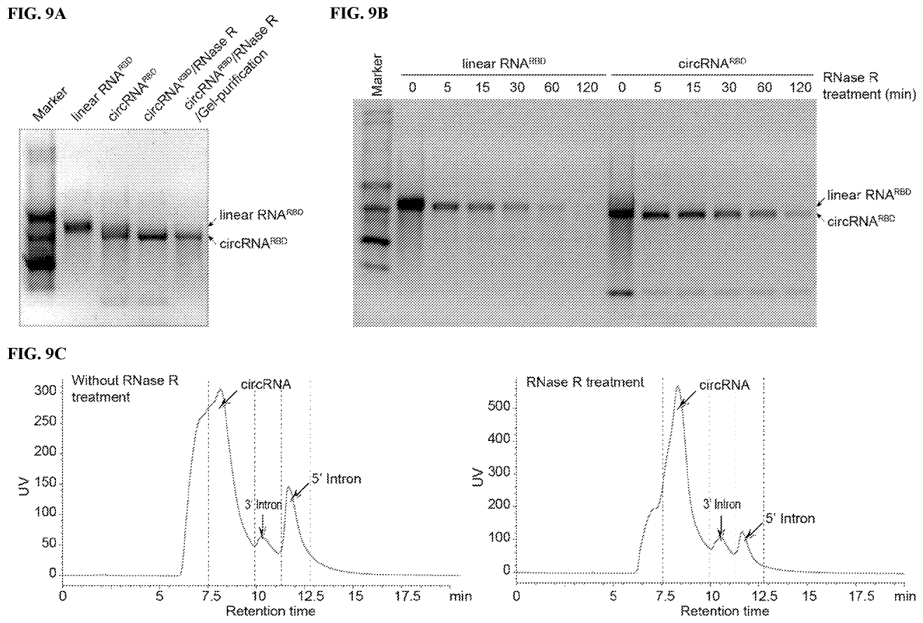

Owing to its covalently closed circular structure, the circRNA^{RBD} migrated faster in electrophoresis (FIG. 9A) and appeared more resistant to exonuclease RNase R than the linear precursor RNA (FIG. 9B). High-performance liquid chromatography (HPLC) showed that the RNase R treatment purged significant amount of the linear precursor RNAs, which was an important step for the purification of the circRNA^{RBD} (FIG. 9C).

To test the secretory expression of RBD antigens produced by circRNA^{RBD}, the purified circRNA^{RBD} was transfected into HEK293T cells. Western blot result demonstrated the ample production of RBD antigens in the supernatant (FIG. 1C). Quantitative ELISA assay showed that the concentration of RBD antigens produced by circRNA^{RBD} reached ˜1400 ng/mL, 600-fold more than the linear precursor RNA (FIG. 1D). The circRNA^{RBD} could also produce RBD antigens in murine NIH3T3 cells, indicating the high compatibility of circRNAs (FIG. 1E). Together, these results demonstrated that abundant secretory RBD antigens could be produced using circRNA^{RBD} in both human and murine cells.

Apart from the Group I ribozyme autocatalysis strategy, T4 RNA ligase was also used to generate circRNA^{RBD} (FIG. 1F). Similarly, western blot and quantitative ELISA results demonstrated that effective expression of RBD antigens in the supernatant, and the antigen concentration reached ˜1000 ng/mL, ˜200-fold more than its linear precursor RNA (FIGS. 1G-1H).

To verify whether the secreted SARS-CoV-2 RBD antigens produced by circRNA^{RBD} were functional, the supernatants of circRNA^{RBD}-transfected cells were used for competition assay using hACE2-overexpressing HEK293 cells (HEK293T-ACE2) and SARS-CoV-2 pseudovirus harboring an EGFP reporter⁵⁰. The secreted SARS-CoV-2 RBD antigens could effectively block SARS-CoV-2 pseudovirus infection (FIG. 1I). Altogether, circRNA^{RBD} showed robust protein expression and high stability, illuminating its potential to be engineered into a novel vaccine platform.

### Example 2: SARS-CoV-2 circRNA^{RBD} Vaccine Induced Sustained Humoral Immune Responses with High-Level Neutralizing Antibodies

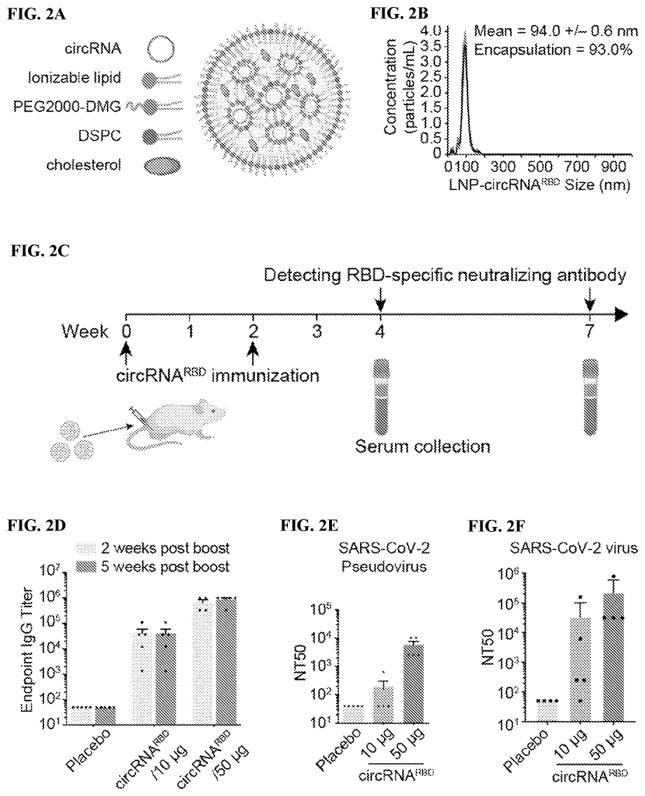

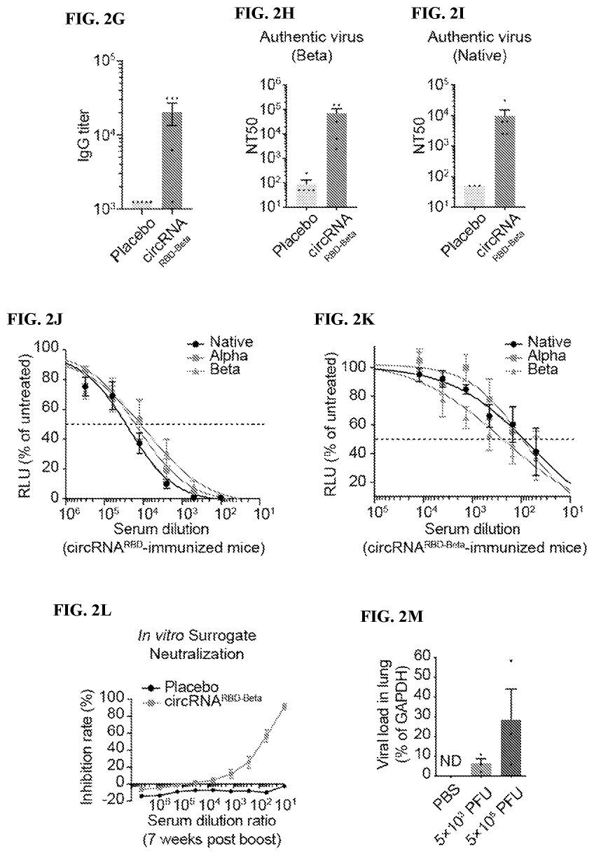

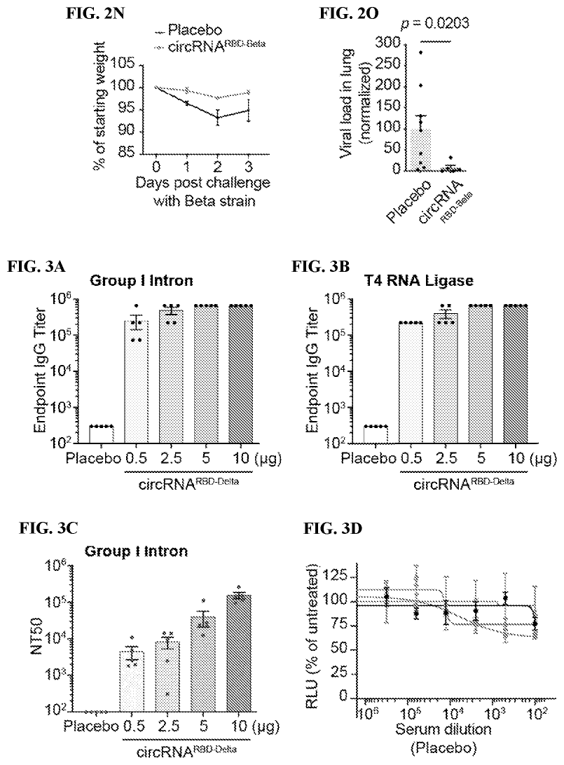

To explore whether circRNA could be developed into a new type of vaccine, the immunogenicity of circRNA^{RBD} encapsulated with lipid nanoparticle in BALB/c mice (FIG. 2A) was then assessed. The circRNA^{RBD} encapsulation efficiency was more than 93%, with an average size of 100 nm in diameter (FIG. 2B). Mice were immunized with LNP-circRNA^{RBD} through intramuscular injection twice with a two-week interval, using a dose of 10 μg or 50 μg per mouse, while empty LNP was used as the placebo control (FIG. 2C). The amount of RBD-specific binding IgG and neutralizing antibodies were evaluated at two or five weeks post boost dose. High titers of RBD-specific IgG were elicited by circRNA^{RBD} in a dose-dependent manner, ˜3×10⁴ and ˜1×10⁶ for each dose and for both 2- and 5-weeks post boost, indicating that circRNA^{RBD} could induce long-lasting antibodies against SARS-CoV-2 RBD (FIG. 2D).

To test the neutralizing capability of serum samples from vaccinated mice, neutralization assay was performed. It is demonstrated that sera from circRNA^{RBD}-vaccinated mice could effectively neutralize both SARS-CoV-2 pseudovirus (FIG. 2E) and authentic SARS-CoV-2 virus (FIG. 2F), with an NT50 of ˜5.6×10³ (FIG. 2E) and an NT50 of ˜2.2×10⁵ (FIG. 2F) in mice (50 μg of circRNA^{RBD}), respectively. The large amount of RBD-specific IgG, potent RBD antigen neutralization, and sustained SARS-CoV-2 neutralizing capacity suggest that circRNA^{RBD} vaccines did induce long-lasting humoral immune responses in mice.

### Example 3: SARS-CoV-2 circRNA^{RBD-Beta} Vaccine-Elicited Antibodies Showed Preferential Neutralizing Activity Against Beta Variant

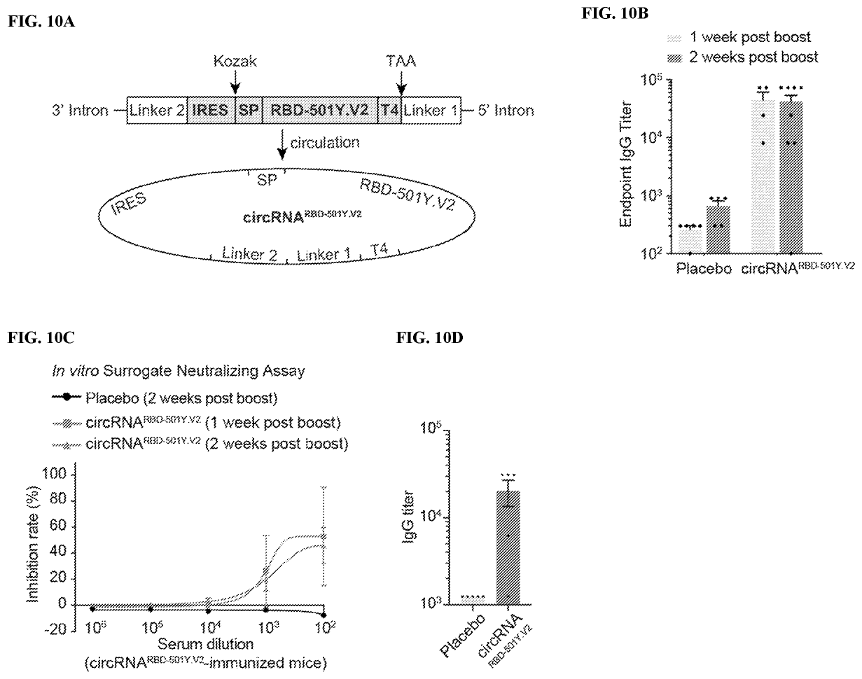

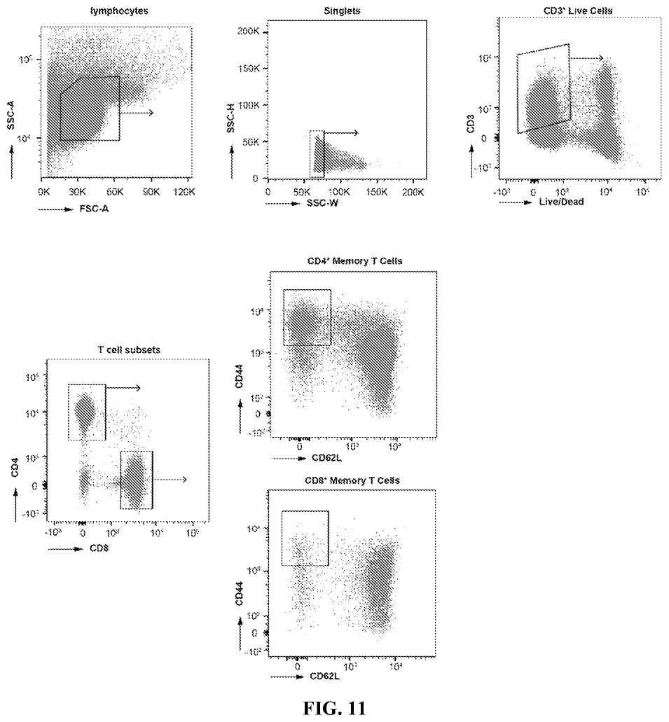

Next, the efficacy of a circRNA vaccine encoding RBD/K417N-E484K-N501Y antigens derived from SARS-CoV-2 Beta variant, termed as circRNA^{RBD-Beta} was evaluated. BALB/c mice were immunized with an i.m. injection of the circRNA^{RBD-Beta} vaccine, followed by a boost at a two-week interval. The immunized mice's sera were collected at 1 and 2 weeks post the boost. ELISA showed that the RBD-Beta-specific IgG endpoint titer could reach ˜7×10⁴ at 2 weeks post boost (FIG. 2G). The surrogate neutralization assay showed that sera of circRNA^{RBD-Beta} immunized mice effectively neutralized RBD antigens (FIG. 10C). SARS-CoV-2 pseudovirus neutralization assay revealed that circRNA^{RBD}-elicited antibodies could effectively neutralize all three viral strains, with the highest neutralizing activity against the D614G strain (FIG. 2H). The circRNA^{RBD-Beta}-elicited antibodies could also neutralize all three pseudoviruses, with the highest neutralizing activity against its corresponding variant, Beta strain (FIG. 2I).

In line with pseudovirus neutralization assay; the serum could effectively neutralize authentic SARS-CoV-2 Beta strain with an NT50 of 7.1×10⁴ (FIG. 2J), and could neutralize authentic SARS-CoV-2 D164G strain less effectively with an NT50 of 9.8×10³ (FIG. 2K). Collectively, circRNA vaccines-elicited antibodies showed the best neutralizing activity against their corresponding variant strains. It's worth noting that both vaccines could neutralize all three strains albeit with variable efficacies. Nevertheless, it's very likely that the updated vaccines based on the mutations of the emerging variants or multivalent vaccines might provide better and more effective protection against SARS-CoV-2 circulating variants, especially the recent Omicron variants with severe antibody escape capability.

### Example 4: circRNA^{RBD-Beta} Vaccine Protected Mice Against the Infection of SARS-CoV-2 Beta Variant

To further evaluate the protective efficacy of SARS-CoV-2 circRNA^{RBD-Beta} vaccines in vivo, the Beta strain for authentic virus challenge experiments were conducted. Consistent with a recent report⁵⁹, the SARS-CoV-2 Beta variant could infect BALB/c mice and replicate in their lungs (FIG. 2L), due to the mutations in spike protein such as K417N, E484Q, and N501Y^{59,60}. Near two months after the boost dose, the titer of RBD-501Y.V2-specific IgG is around 2×10⁴ (FIG. 10B), and the serum showed significant neutralizing capacity against RBD-Beta antigens (FIG. 2M). Then each immunized mouse was challenged with 5×10⁴ PFU of authentic SARS-CoV-2 B.1.351/501Y.V2 strain via the intranasal route at 7 weeks post the boost dose, and the lung tissues were collected 3 days after challenge for detecting viral RNA. It is found that the mice in the placebo group underwent increased weight loss compared with vaccinated mice (FIG. 2N). Consistently, virus titers in the lungs of vaccinated mice were significantly decreased compared with those who received placebos (FIG. 2O). These results indicated that the circRNA^{RBD-Beta}, vaccine could effectively protect the mice against the infection by SARS-CoV-2 Beta variant.

### Example 5: circRNA^{RBD-Delta} Vaccine Induced Potent Neutralizing Antibodies Against SARS-CoV-2 Delta Variant

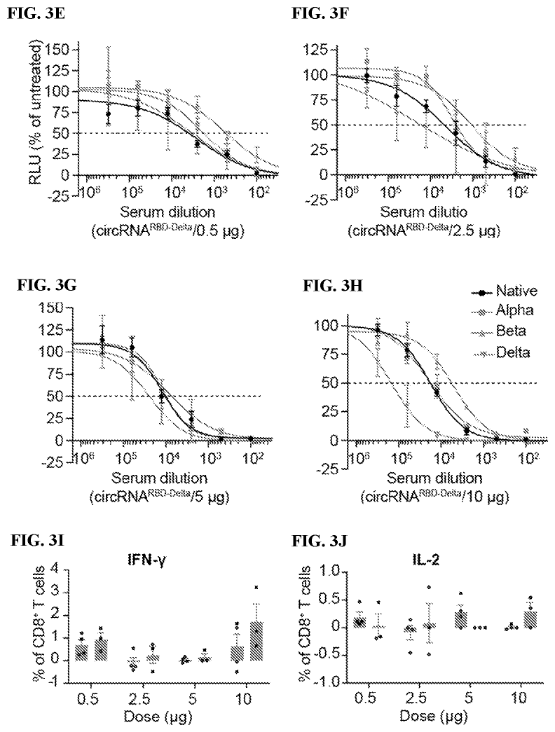

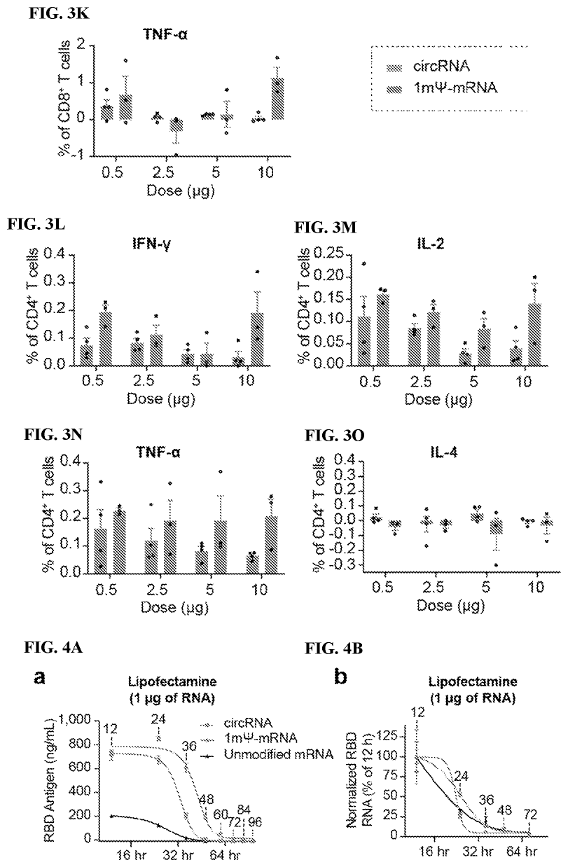

Currently, the SARS-CoV-2 Delta variant is the major pandemic strain worldwide. Similar to the Beta variant, the Delta variant also partially escapes the antibodies produced in the survivors or vaccinees. Therefore, the development of updated vaccines against Delta variant is urgently demanded. Both Group I ribozyme autocatalysis and T4 RNA ligation strategies were adopted to generate circRNA^{RBD-Delta} encoding RBD antigens of Delta variant. The circRNA^{RBD-Delta} was then encapsulated with LNP (PRECISION NANOSYSTEMS) to generate LNP-circRNA^{RBD-Delta} vaccine. Mice were immunized i.m. with 0.5 μg, 2.5 μg, 5 μg or 10 μg of LNP-circRNA^{RBD-Delta} vaccines twice at a 2-week interval, while empty LNP was used as the placebo control. Two weeks post the boost dose, the serum samples from immunized mice were collected for the detection of RBD-specific antibodies. Both the Group I ribozyme autocatalysis strategy or T4 RNA ligation strategy-based circRNA^{RBD-Delta} vaccines could induced high titers of IgG binding antibodies in mice (FIGS. 3A-3B). The sera from circRNA^{RBD-Delta}-vaccinated mice could effectively neutralize the SARS-CoV-2 Delta pseudovirus in a dose-dependent manner, and the NT50 could even reached ˜2×10⁵ for the dose of 10 μg (FIG. 3C).

Importantly, the sera from circRNA^{RBD-Delta}-immunized mice could also effectively neutralize other variants, including Native strain, Alpha and Beta variants, but the neutralizing capability were different. The sera from circRNA^{RBD-Delta}-immunized mice exhibited the high neutralizing activity against the corresponding Delta variant. The neutralizing activity against the Native strain and Alpha variant partly decreased (FIGS. 3D-3E), and the Beta variant worst escaped the sera of circRNA^{RBD-Delta}-immunized mice, especially at the dose of 10 μg (FIG. 3E). The neutralizing capability against these four SARS-CoV-2 variants elevated with the increase of dose for vaccination from 0.5 μg to 5 μg. However, when the dose further increased from 5 μg to 10 μg, the neutralizing activity against Native strain, Alpha or Beta variants failed to increase (FIG. 3E). But the neutralizing activity against the corresponding Delta variants continued to increase, almost 10-fold (FIG. 3E). In line with the above results about circRNA^{RBD-Beta} (FIGS. 2H-2K), these results strongly indicated that developing updated vaccines according to the corresponding mutations in emerging variants might provide more effective protection than the current strategy that used the original SARS-CoV-2 vaccine as the third booster.

### Example 6: circRNA^{RBD-Delta} Vaccine Elicited T Cell Immune Responses

B cells, CD4⁺ T cells, and CD8⁺ T cells are three pillars of adaptive immunity, and they mediated effector functions that have been associated with the control of SARS-CoV-2 in both non-hospitalized and hospitalized cases of COVID-19 (Sette and Crotty, 2021).

To probe and compare CD4⁺ and CD8⁺ T cell immune responses elicited by circRNA^{RBD-Delta} with 1mΨ-modified mRNA^{RBD-Delta} (1mΨ-mRNA^{RBD-Delta}) vaccine, the splenocytes of mice immunized with circRNA^{RBD-Delta} vaccine or 1mΨ-mRNA^{RBD-Delta} vaccine were collected and stimulated with SARS-CoV-2 RBD-Delta pooled peptides (Table B below), and cytokine-producing T cells were quantified by intracellular cytokine staining among effector memory T cells (Tem, CD44⁺CD62L⁻) (FIG. 11). Stimulated with RBD peptide pools, the CD8⁺ T cells from mice immunized with circRNA^{RBD-Delta} vaccine or 1m_{Ψ}U-mRNA^{RBD-Delta} vaccine producing interferon-γ (IFN-γ), tumor necrosis factor (TNF-α), and interleukin-2 (IL-2) were detected (FIGS. 3F-3H), indicating the RBD-specific CD8⁺ T cell responses elicited by both the circRNA^{RBD-Delta} and 1m_{Ψ}U-mRNA^{RBD-Delta} vaccines. The CD4⁺ T cells of mice immunized with circRNA^{RBD-Delta} vaccine or 1m_{Ψ}U-mRNA^{RBD-Delta} vaccine induced strong IFN-γ, TNF-α, and IL-2 responses, but minimal IL-4 responses (FIGS. 3I-3L), indicating that both circRNA^{RBD-Delta} and 1mΨ-mRNA^{RBD-Delta} vaccines induced the Th1-biased T cell immune responses, whereas 1mΨ-mRNA^{RBD-Delta} vaccines exhibited a litter bit stronger CD4⁺ T cell responses than circRNA^{RBD-Delta} vaccine (FIGS. 3I-3K)

TABLE B
The peptide sequences of RBD-Delta antigens

| | | Length | SEQ ID |
|---|---|---|---|
| Name | Sequence | (amino acid) | NO |
| S-45 | GIYQTSNFRVQPTESIVR | 18 | 43 |
| S-46 | RVQPTESIVRFPNITNL | 17 | 44 |
| S-47 | IVRFPNITNLCPFGEVF | 17 | 45 |
| S-48 | TNLCPFGEVENATRFASV | 18 | 46 |
| S-49 | VFNATRFASVYAWNRKRI | 18 | 47 |
| S-50 | SVYAWNRKRISNCVADY | 17 | 48 |
| S-51 | KRISNCVADYSVLYNSA | 17 | 49 |
| S-52 | ADYSVLYNSASFSTFKCY | 18 | 50 |
| S-53 | SASFSTFKCYGVSPTKL | 17 | 51 |
| S-54 | KCYGVSPTKLNDLCFTNV | 18 | 52 |
| S-55 | KLNDLCFTNVYADSFVIR | 18 | 53 |
| S-56 | NVYADSFVIRGDEVRQIA | 18 | 54 |
| S-57 | IRGDEVRQIAPGQTGKIA | 18 | 55 |
| S-58 | IAPGQTGKIADYNYKL | 16 | 56 |
| S-59 | GKIADYNYKLPDDFTGCV | 18 | 57 |
| S-60 | KLPDDFTGCVIAWNSNNL | 18 | 58 |
| S-61 | CVIAWNSNNLDSKVGGNY | 18 | 59 |
| S-62 | NLDSKVGGNYNYRYRLFR | 18 | 60 |
| 2S-63 | NYNYRYRLFRKSNLKPF | 17 | 61 |
| S-64 | LFRKSNLKPFERDISTEI | 18 | 62 |
| S-65 | PFERDISTEIYQA | 13 | 63 |
| S-66 | RDISTEIYQAGSKPCNGV | 18 | 64 |
| S-67 | YQAGSKPCNGVEGENCYF | 18 | 65 |
| S-68 | NGVEGFNCYFPLQSYGF | 17 | 66 |
| S-69 | CYFPLQSYGFQPTNGVGY | 18 | 67 |
| S-70 | GFQPTNGVGYQPYRVVVL | 18 | 68 |
| S-71 | GYQPYRVVVLSFELLHA | 17 | 69 |
| S-72 | VVLSFELLHAPATVCGPK | 18 | 70 |
| S-73 | HAPATVCGPKKSTNLVK | 17 | 71 |
| S-74 | GPKKSTNLVKNKCVNFNF | 18 | 72 |
| S-75 | VKNKCVNFNFNGLTGTGV | 18 | 73 |

### Example 7: circRNA Vaccine Possessed Higher Antigen-Encoding Efficiency than mRNA Vaccine

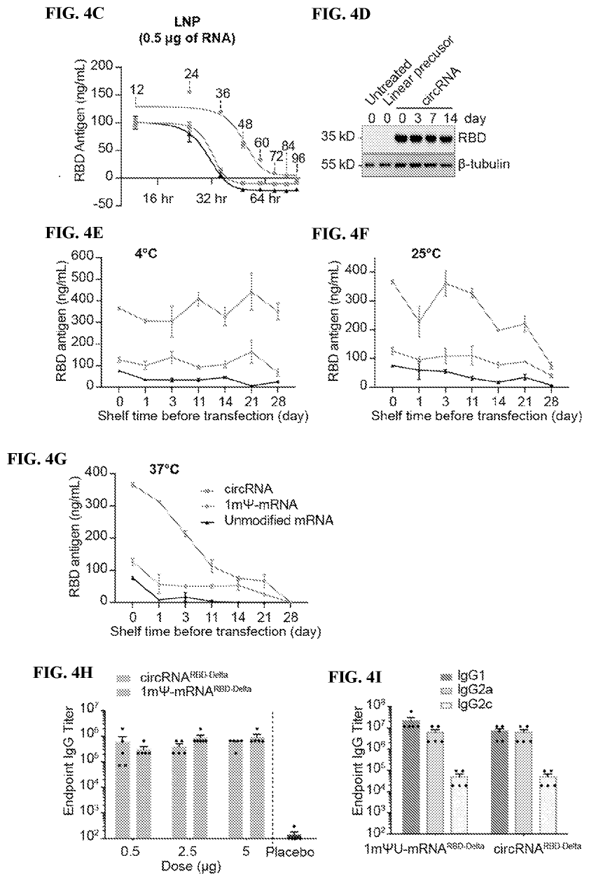

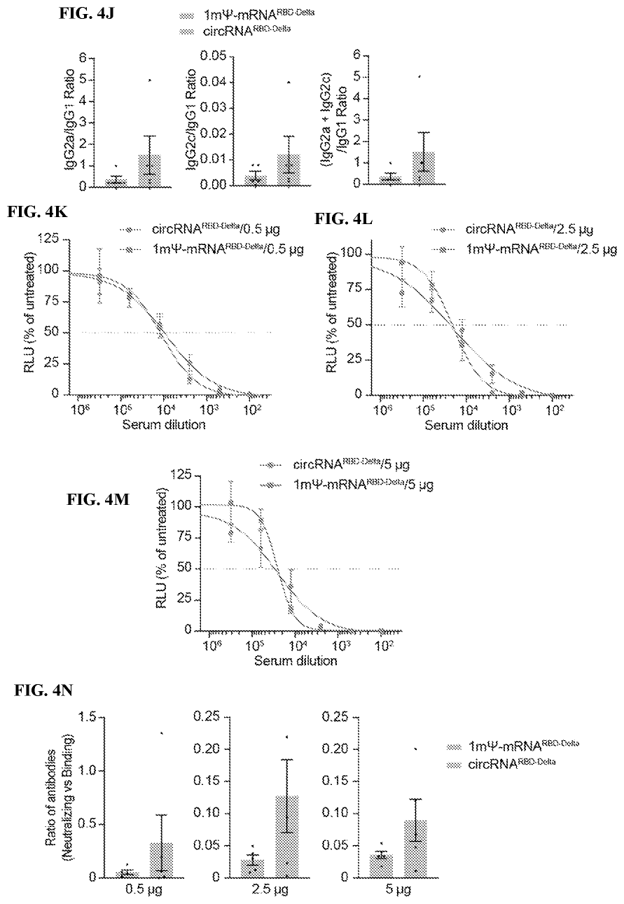

Owing to its covalent closed circular structure, the circRNA is reportedly more stable than mRNA (Fischer and Leung, 2017). To test whether the stability of circRNA vaccine could confer higher antigen-encoding efficiency, 1mΨ modified mRNA, termed 1mΨ-mRNA, and unmodified mRNA both of which contains the same RBD-encoding sequence as the circRNA were generated. The purified circRNA, 1mΨ-mRNA and unmodified mRNA were transfected into the HEK239T cells, respectively. Cell supernatants were collected to measure the abundance of RBD antigen at different time point from 12 to 96 hours. ELISA results showed that the circRNA could not only produce much more level of RBD antigens at each time point, but also generate RBD antigens more consistently than both 1mΨ-mRNA and unmodified mRNA (FIG. 4A). The RT-qPCR results demonstrated that circRNAs were more stable than 1mΨ-mRNA and unmodified mRNA (FIG. 4B). Similarly, the LNP-encapsulated circRNA also produced much more and more durable RBD antigens than both 1mΨ-mRNA and unmodified-mRNA (FIG. 4C). Interestingly, the LNP-encapsulation might improve the antigen-encoding efficiency of unmodified mRNA to the comparable level of 1mΨ-mRNA, but both of them were still much lower than circRNAs (FIG. 4C).

A thermostable RNA vaccine is highly desirable for efficient vaccine distribution. It is found that the purified circRNA could be readily expressed without detectable loss even after two weeks of storage at room temperature (˜25° C.) (FIG. 4D), highlighting its remarkable thermal stability. Moreover, to compare the thermostability of LNP-circRNA formulation with LNP-1mΨ-mRNA and LNP-unmodified-mRNA, the LNP-encapsulated circRNA, 1mΨ-mRNA or unmodified-mRNA was stored at 4° C., room temperature (˜25° C.) or 37° C. for 1, 3, 11, 14, 21 and 28 days before transfection. ELISA results showed that showed the antigen-encoding efficiency of circRNA was much higher than that of the other two mRNA groups at 4° C., 25° C. or 37° C. At 4° C., nearly no reduction of RBD antigens produced by LNP-encapsulated circRNA could be detected from 1-28 days (FIG. 4E), while the reduction of RBD antigens produced by LNP-encapsulated 1mΨ-mRNA or unmodified-mRNA started on day 21 and day 28, respectively (FIG. 4E). When the storage temperature elevated to 25° C. or 37° C., the stability of LNP-encapsulated circRNA, 1mΨ-mRNA and unmodified-mRNA was reduced, especially at 37° C. (FIGS. 4F-4G). Altogether, these results indicated that the deterioration effects did occur with extended shelf life and high temperature, but the LNP-encapsulated circRNA exhibited high stability at 4° C. (FIGS. 4E-4G).

### Example 8: circRNA Vaccine Elicited Higher Proportion of Neutralizing Antibodies and Stronger Th1-Biased T Cell Immune Responses than mRNA Vaccine

Currently, there are two kinds of mRNA widely inoculated, mRNA-1273 (Moderna) and BNT162b2 (Pfizer/BNT), both of which contain modified 1mΨ. Given that circRNA^{RBD-Delta} vaccine possess higher stability and antigen-encoding efficiency, it is unknown whether the LNP-circRNA vaccine exhibited superior immunogenicity to LNP-1mΨ-mRNA^{RBD-Delta} vaccine. Firstly, the balance of Th1 and Th2 immune responses between circRNA^{RBD-Delta} vaccine and 1mΨ-mRNA^{RBD-Delta} vaccine was compared, because Th2-biased immune responses might induce vaccine-associated enhanced respiratory disease (VAERD). Eliciting strong Th1-biased immune responses is a major safety consideration for SARS-CoV-2 vaccine design. Therefore, the endpoint titer of IgG2a/c and IgG1, which represents Th1 and Th2 immune responses, elicited by circRNA^{RBD-Delta} vaccine or 1mΨ-mRNA^{RBD-Delta} vaccine was compared. ELISA demonstrated that although the circRNA^{RBD-Delta} vaccine induced comparable level of total IgG to 1mΨ-mRNA^{RBD-Delta} vaccine (FIG. 4H), circRNA^{RBD-Delta} vaccine elicited much higher ratio of IgG2a/IgG1, IgG2c/IgG1 or (IgG2a+IgG2c)/IgG1 than 1mΨ-mRNA^{RBD-Delta} vaccine (FIGS. 4I-4J), indicating that circRNA^{RBD-Delta} vaccine tended to induce stronger Th1-biased immune responses, about ˜7-fold higher than mRNA vaccine.

Antibody-mediated enhancement (ADE) of infection by virus-specific antibodies is another potential concern for vaccines, which has been reported for some virus infection including Zika, Dengue virus and coronaviruses. Previous research has been reported that the virus-binding antibodies without neutralizing activity elicited by virus infection or vaccination possibly caused the ADE effects, especially for those virus with different serotypes. Therefore, the differences in the ratio of neutralizing antibodies to binding antibodies between circRNA^{RBD-Delta} vaccine and 1mΨ-mRNA^{RBD-Delta} vaccine were investigated. It is found that although circRNA^{RBD-Delta} exhibited equal neutralizing capability compared to 1mΨ-mRNA^{RBD-Delta} vaccine (FIGS. 4K-4M), circRNA^{RBD-Delta} vaccine could induce higher proportion of neutralizing antibodies than 1mΨ-mRNA^{RBD-Delta} vaccine at different dose (0.5 μg. 2.5 μg and 5 μg) (FIG. 4N), indicating that much lower ratio of virus-binding but non-neutralizing antibodies were elicited by circRNA^{RBD-Delta} vaccine in mice. With this unique feature, the circRNA platform might have important superiority to meet the potential ADE effects and virus variation.

Collectively, these results demonstrated that circRNA^{RBD-Delta} could elicit higher proportion of neutralizing antibodies and stronger Th1-biased T cell immune responses than 1mΨ-mRNA^{RBD-Delta} vaccine.

### Example 9: circRNA^{RBD} Vaccine Elicited Potent Neutralizing Antibodies and Th1-Biased Immune Responses in Rhesus Macaques

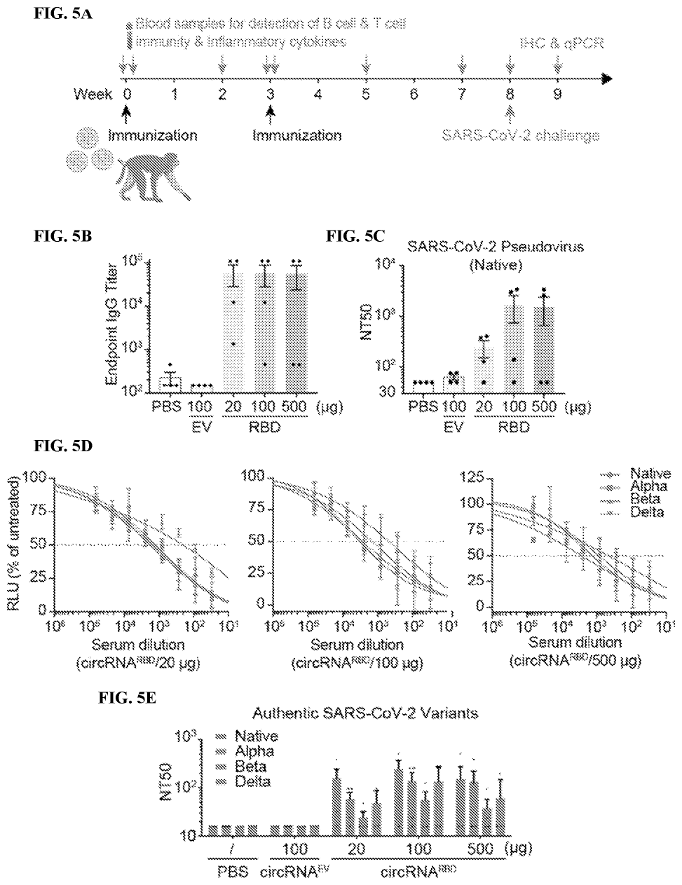

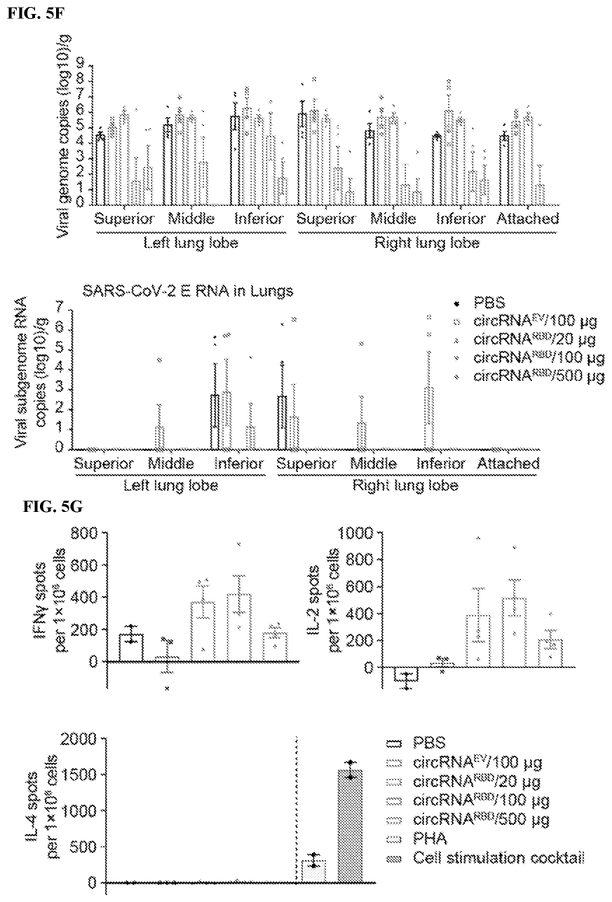

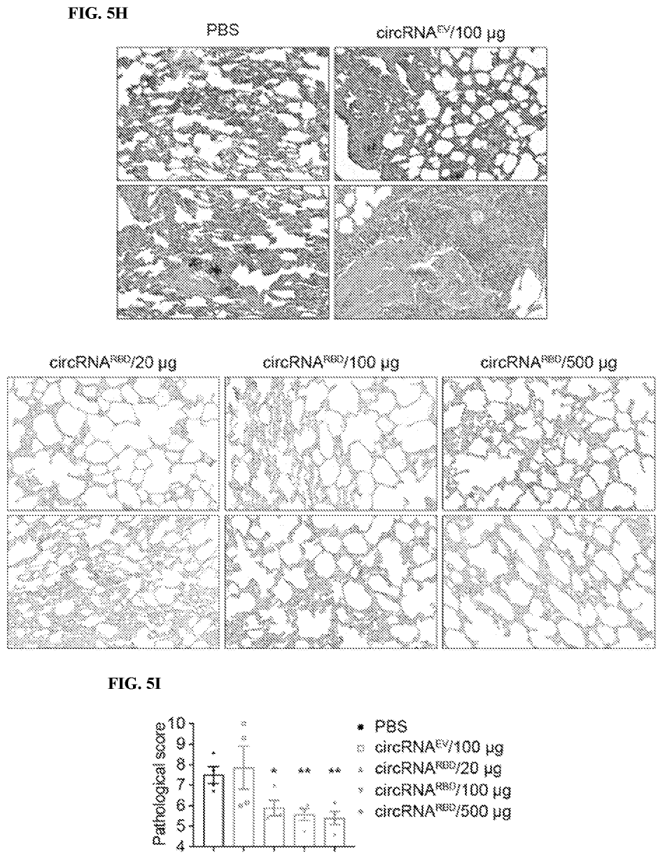

To further assess the immunogenicity of circRNA vaccine in non-human primates, groups of 2.5-4 year old rhesus macaques were immunized i.m. with 20 μg, 100 μg or 500 μg of LNP-circRNA^{RBD} vaccine, or 100 μg of LNP-circRNA^{EV} control (circRNA empty vector without encoding antigens), or PBS control on Days 0 and 21 (FIG. 5A). The total RBD-specific IgG binding antibodies and neutralizing antibodies were measured using the plasma of rhesus macaques at two weeks post the boost dose (FIG. 5A). The ELISA result showed that the endpoint IgG titer reached ˜5.8×10⁴ (20 μg or 100 μg dose level) and ˜5.5×10⁴ (500 μg dose level) for LNP-circRNA^{RBD} vaccine, while the plasma samples from LNP-circRNA^{EV} or PBS-immunized rhesus macaques had no detectable RBD-specific IgG binding antibodies (FIG. 5B).

Next, the virus entry inhibition ability of plasma from LNP-circRNA^{RBD} vaccine-immunized rhesus macaques were detected with both the VSV-based SARS-CoV-2 pseudovirus and authentic SARS-CoV-2 neutralization assay. The pseudovirus neutralization assay showed that the NT50 mean value reached ˜241 (20 μg dose level), ˜1656 (100 μg dose level) and ˜1532 (500 μg dose level) (FIG. 5C). Authentic SARS-CoV-2 neutralization assay showed that NT50 mean value reached ˜156 (20 μg dose level), ˜244 (100 μg dose level) and ˜152 (500 μg dose level), respectively (FIG. 5D). Both the pseudotyped and authentic SARS-CoV-2 neutralization assay indicated that the 100 μg dose level could elicit high-level neutralizing antibodies, and higher dose level (500 μg) didn't further to elevate the level of neutralizing antibodies (FIGS. 5C-5D).

In consideration of the circulating variants of SARS-CoV-2, the cross-neutralizing assay using the plasma samples from the immunized rhesus macaques was performed. Both the pseudotyped and authentic SARS-CoV-2 neutralization assay showed that the LNP-circRNA^{RBD} vaccine-immunized rhesus macaque plasma could efficiently inhibit the corresponding native strain, while Alpha and Delta variants could also be inhibited, but slightly escaped compared to the native strain (FIGS. 5D, 5E). Beta variant could also be neutralized by the plasma samples from LNP-circRNA^{RBD} vaccine-immunized rhesus macaque, but it partly escaped (FIGS. 5D, 5E).

The RBD-specific T cell responses elicited by the LNP-circRNA^{RBD}, LNP-circRNA^{EV} or PBS in rhesus macaques were detected using the peripheral blood mononuclear cells (PBMCs) collected on the day before challenge with SARS-CoV-2. ELISpot assay demonstrated evident IFN-γ and IL-2 responses, but nearly un-detectable IL-4 responses in the LNP-circRNA^{RBD} vaccine-immunized rhesus macaque (FIG. 5F), indicating a Th1-biased T cell immune response elicited by LNP-circRNA^{RBD} vaccine in rhesus macaque.

### Example 10: circRNA^{RBD} Vaccine Protected the Rhesus Macaques Against SARS-CoV-2 Infection

Five weeks post the boost dose, the rhesus macaques immunized with LNP-circRNA^{RBD} vaccine (20 μg, 100 μg or 500 μg), LNP-circRNA^{EV} (100 μg) or PBS control, were challenged with 1×10⁶ plaque forming units of SARS-CoV-2 native strain via intranasal and intratracheal routes, as described previously. On the days post virus challenge, the challenged rhesus macaques were euthanized at 7 dpi, and the lung tissues were collected and analyzed for viral loads and histopathological assays. The reverse-transcription quantitative PCR (RT-qPCR) using primers targeting SARS-CoV-2 genomic RNA (N protein region) demonstrated that the rhesus macaques immunized with 100 μg or 500 μg of LNP-circRNA^{RBD} vaccine were highly protected against SARS-CoV-2 infection because the SARS-CoV-2 genomic RNA copies were reduced nearly 1000-fold comparing to the LNP-circRNA^{EV} or PBS control (FIG. 5G). To detect the viral loads of actively replicative SARS-CoV-2, qPCR used primers targeting SARS-CoV-2 sub-genomic RNA (E protein region) was performed. The quantitative PCR showed that all the 20 μg, 100 μg and 500 μg of LNP-circRNA^{RBD}-immunized rhesus macaques had nearly no detectable viral sub-genomic RNA in the lung tissues (FIG. 5G).

Moreover, further histopathological examination demonstrated that the LNP-circRNA^{RBD} (20 μg, 100 μg or 500 μg) vaccine-immunized rhesus macaques were well protected against the pneumonia caused by SARS-CoV-2 infection, because very mild pneumonia was observed in the LNP-circRNA^{RBD}-immunized groups, especially the 100 μg or 500 μg dose group (FIG. 5H). In contrast, severe pneumonia symptoms were observed in the lungs of LNP-circRNA^{EV} or PBS control group, such as the local pulmonary septal thickening, moderate hemorrhage in the pulmonary septals, a large number of scattered dust cells and massive inflammation cells infiltration (FIG. 5H). Pathological score further demonstrated that the LNP-circRNA^{RBD} immunization could significantly protect the rhesus macaques against the pneumonia caused by SARA-CoV-2 infection (FIG. 5I), which might be a synergy between the humoral immune responses and T cell immune responses elicited by the LNP-circRNA^{RBD} vaccine (FIG. 5J).

### Example 11: circRNA Vaccine Caused No Obvious Clinical Signs of Illness in Rhesus Macaques

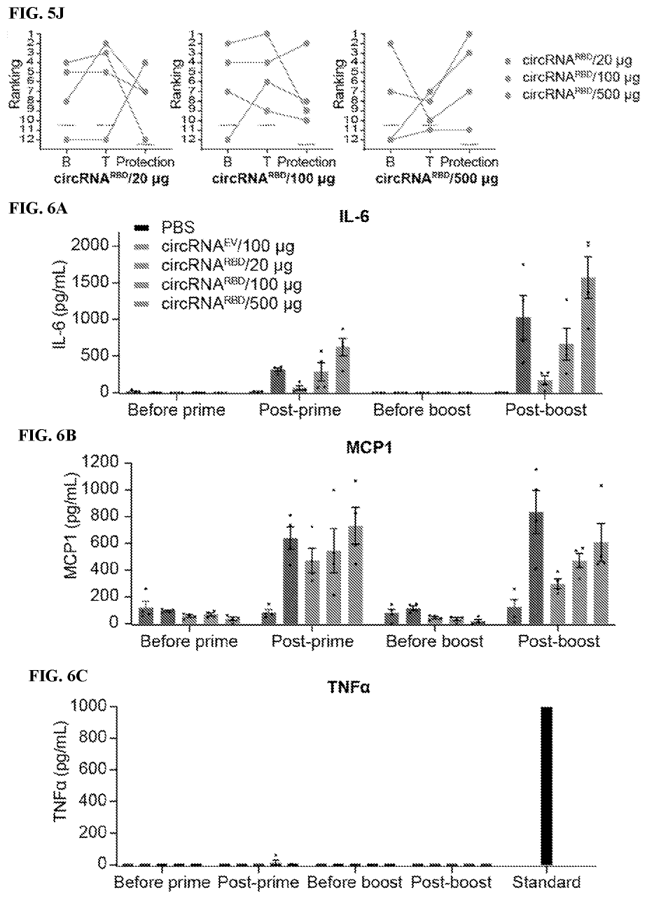

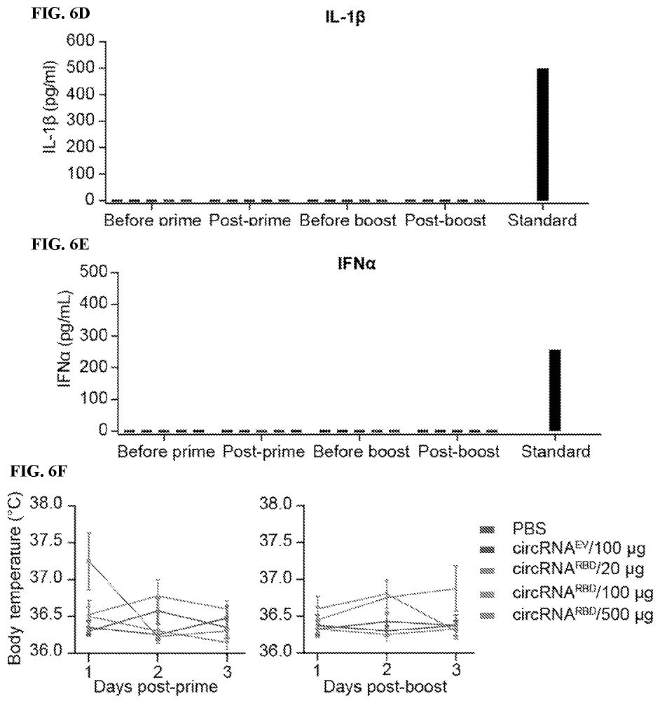

Apart from the efficacy of circRNA vaccine in rhesus macaques, the immunogenicity and safety of circRNA vaccine is also important for a new-typed vaccine platform. To evaluate the immunogenicity and safety of circRNA vaccine in rhesus macaques, some physiological and biochemical indicators were monitored, which included adverse effects, cytokines indicating innate immune activation, body weight, body temperature and blood routine examination. No severe clinical adverse effects were observed following prime and boost. ELISA results showed that circRNA^{RBD} vaccines induced high levels of IL-6 and MCP-1 (FIGS. 6A, 6B), while the TNF-α, IL-1β and IFN-α were nearly undetectable (FIGS. 6C-6E). Body temperature of immunized rhesus macaques was continuous monitored for 3 days after prime and boost, which showed that body temperature of the circRNA^{RBD} circRNA^{EV} or PBS-immunized rhesus macaques were within the range of normal physiological indicators (FIG. 6F). Collectively, our study provided the preliminary proof of safety for the circRNA vaccine platform in non-human primates.

### Example 12: circRNA Vaccines Induced Reduced Neutralizing Antibodies Against SARS-CoV-2 Omicron Variant

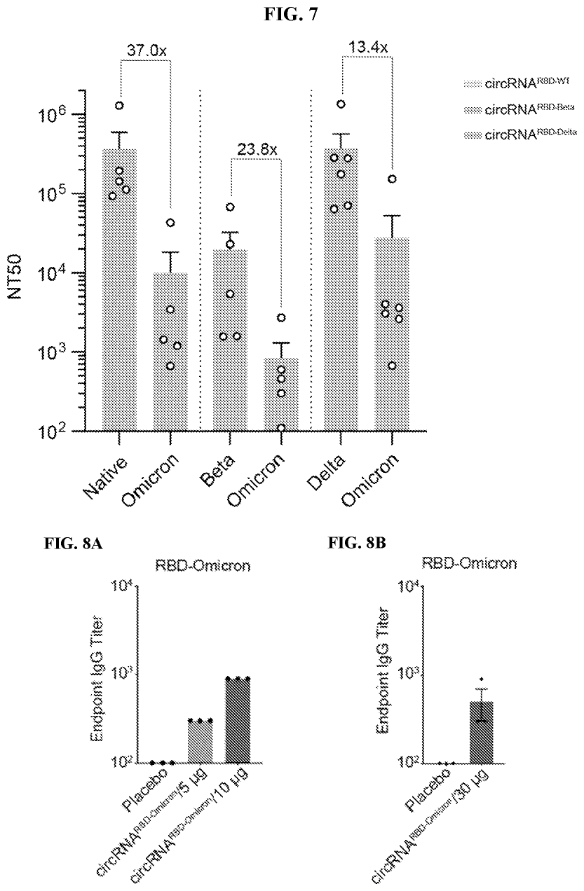

Currently, the SARS-CoV-2 Omicron variant is rapidly spreading worldwide and causes significant humoral immune evasion in vaccinees who got vaccines against SARS-CoV-2 native strain. Therefore, the evaluation of our vaccines, especially of Beta and Delta variants against Omicron variant must be done. As shown in the above results, Group I ribozyme autocatalysis was adopted to generate circRNA^{RBD} vaccine encoding RBD antigens of SARS-CoV-2 native strain and 2 variants (Beta and Delta). To evaluate the efficiency of these vaccines against Omicron Variant. The serum sample of mice immunized with 10 μg of LNP-circRNA^{RBD/RBD-Beta/RBD-Delta} vaccines were collected for the detection of neutralizing antibodies against Omicron variant by pseudovirus neutralization assay. It is found that the sera from circRNA^{RBD/RBD-Beta/RBD-Delta}-vaccinated mice could neutralize the SARS-CoV-2 Omicron pseudovirus but were all strongly escaped (FIG. 7). As for NT50 determined by pseudovirus neutralization assay, sera of circRNA^{RBD} group have ˜37.0 times reduction and sera of circRNA^{RBD-Beta} group have ˜23.8 times reduction (FIG. 7). While the neutralizing activity of sera of circRNA^{RBD-Delta} was much better preserved (˜13.4 times reduction) (FIG. 7).

### Example 13: circRNA^{RBD-Omicron} Vaccine Induced SARS-CoV-2 Omicron Variant-Specific Antibodies

The circRNA^{RBD-Omicron} was encapsulated with LNP to generate LNP-circRNA^{RBD-Delta} vaccine. Mice were immunized i.m. with 5 μg or 10 μg of LNP-circRNA^{RBD-Omicron} vaccines twice at a 1-week interval, while empty LNP was used as the placebo control. Four days post the boost dose, the serum samples from immunized mice were collected for the detection of RBD-specific antibodies. The circRNA^{RBD-Omicron} vaccines could induced Omicron variant-specific antibodies with the endpoint titer of 300 (5 μg dose) and 900 (10 μg dose) (FIG. 8A). Besides, mice were also immunized i.m. with 1-dose 30 μg of LNP-circRNA^{RBD-Omicron} vaccines (FIG. 8B), while empty LNP was used as the placebo control. Eleven days post the boost dose, the serum samples from immunized mice were collected for the detection of RBD-specific antibodies. The circRNA^{RBD-Omicron} vaccines could induced Omicron variant-specific antibodies with the endpoint titer of 500 (FIGS. 8A-8B).

### Example 14: circRNA^{RBD-Delta} Vaccines Elicited High-Level Neutralizing Antibodies Against Omicron Variant

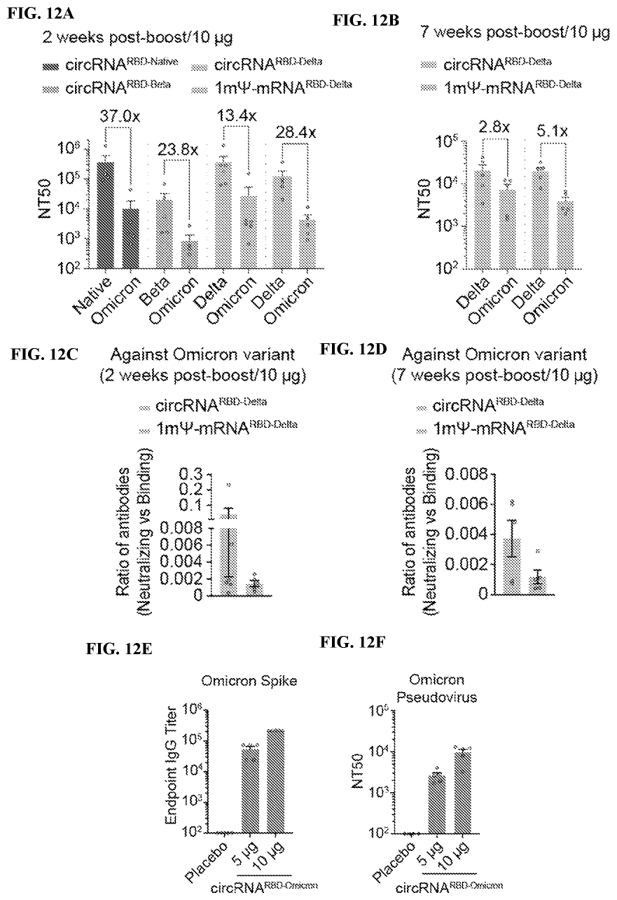

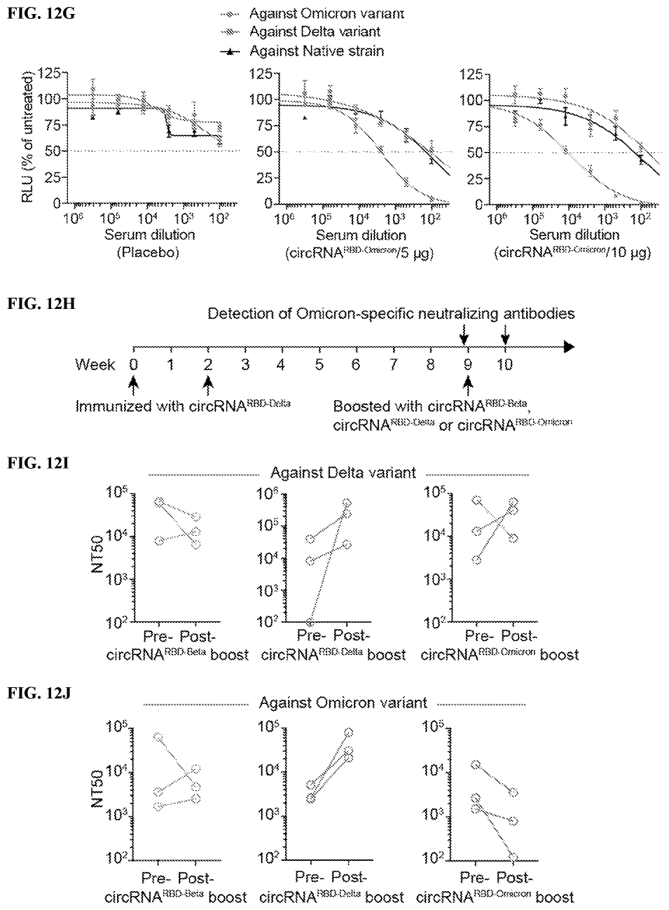

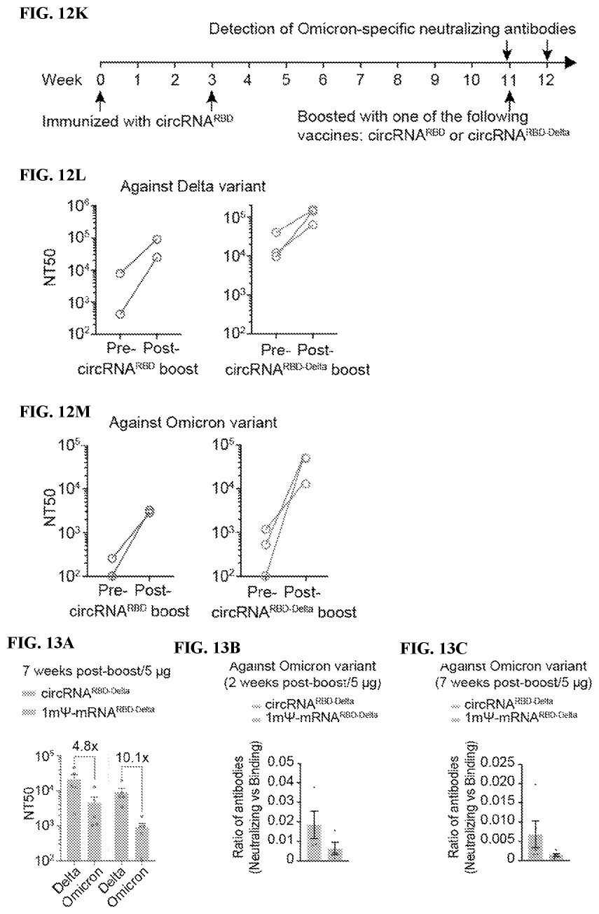

Very recently, the SARS-CoV-2 Omicron variant spreads quickly, which contained more than 30 mutations in the Spike region and escaped most of the previously reported neutralizing antibodies and the sera of vaccinees who received two-dose original SARS-CoV-2 vaccine. To cope with the Omicron emergency, the neutralizing activity of the above circRNA^{RBD}, circRNA^{RBD-Beta} and circRNA^{RBD-Delta} vaccines against Omicron variant was first tested. The pseudovirus neutralization assay showed that the sera of the three circRNA vaccine-immunized mouse could neutralize Omicron variant, especially the circRNA^{RBD-Delta} vaccine with the NT50 of over 2×10⁴ (FIG. 12A). Omicron variant escaped the circRNA^{RBD} vaccine-immunized mouse sera most seriously with a 37-fold drop of neutralizing capability, while the circRNA^{RBD-Delta} vaccine showed much better retention of neutralizing activity with only a 13.4-fold drop (FIG. 12A), indicating that the on-going SARS-CoV-2 vaccines against the Delta variant might still effectively protect against the infection of Omicron variant.

Besides, the efficacy against Omicron variant between circRNA^{RBD-Delta} vaccine and 1mΨ-mRNA^{RBD-Delta} vaccine was also compared. Both the short-term (2 weeks post boost) and long-term (7 weeks post boost) of 10 μg circRNA^{RBD-Delta} vaccine elicited more efficient neutralizing antibodies against Omicron variant, which had higher NT50 value and better retention of neutralizing activity (FIGS. 12A-12B). The NT50 value of the circRNA^{RBD-Delta} vaccine-elicited neutralizing antibodies reached 2×10⁴ at 2 weeks post boost or 4×10³ at 7 weeks post boost, while the NT50 value of the 1mΨ-mRNA^{RBD-Delta} vaccine-elicited neutralizing antibodies reached ˜4×10³ at 2 weeks post boost or 9×10² at 7 weeks post boost (FIGS. 5A-5B). Similar to the above results (FIGS. 12H-12K), it is found that circRNA^{RBD-Delta} vaccine elicited higher proportion of neutralizing antibodies against Omicron variant than 1mΨ-mRNA^{RBD-Delta} vaccine at both 2 weeks post boost (short-term) and 7 weeks post boost (long-term) (FIGS. 12C-12D, FIGS. 13A-13C), indicating the potential superiority of circRNA vaccine against variable variants due to high proportion of neutralizing antibody.

### Example 15: circRNA^{RBD-Omicron} Vaccine Elicited High-Level Neutralizing Antibodies Against Omicron Variant

Besides, a new circRNA vaccine, termed circRNA^{RBD-Omicron} vaccine, encoding the RBD antigens of SARS-CoV-2 Omicron variant was developed. Mice were immunized i.m. with 5 μg or 10 μg of LNP-circRNA^{RBD-Omicron} vaccines twice at a 2-week interval, while empty LNP was used as the placebo control. One week post the 2nd dose, the serum samples from immunized mice were collected for the detection of Omicron RBD/Spike-specific antibodies. The circRNA^{RBD-Omicron} vaccines could induced Omicron Spike-specific antibodies with the endpoint titer of ˜6×10⁴ for 5 μg dose and ˜2×10⁵ for 10 μg dose (FIG. 12E). The sera from circRNA^{RBD-Omicron}-vaccinated mice could effectively neutralize the SARS-CoV-2 Omicron pseudovirus in a dose-dependent manner, and the NT50 could reached ˜3×10³ for the dose of 5 μg and ˜1×10⁴ for the dose of 10 μg (FIG. 12F), while the sera from circRNA^{RBD-Omicron}-vaccinated mice exhibited litter neutralizing activity against the SARS-CoV-2 Delta variant and original SARS-CoV-2 strain (FIG. 12G).

### Example 16: 3^{rd} Booster of circRNA^{RBD-Delta} Vaccine Remarkably Elevated the Neutralizing Antibodies Against Omicron Variant

Besides, to verified whether the additional immunization of 3^{rd} booster of circRNA vaccine after the previous two-dose immunization, could elicited effective neutralizing antibodies against Omicron variant. Mice immunized with 2 doses of circRNA^{RBD-Delta} vaccines received 3^{rd} booster of circRNA^{RBD-Beta}, circRNA^{RBD-Delta} or circRNA^{RBD-Omicron} vaccines at 7 weeks post the 2^{nd} dose (FIG. 12H). The neutralizing antibodies against the Omicron variant pseudovirus were detected at 1 week post the 3^{rd} boost (FIG. 12H). The results showed that the 3^{rd} booster of the same circRNA^{RBD-Delta} vaccine after 2-dose circRNA^{RBD-Delta} vaccine immunization remarkably elevated the neutralizing capability against Delat and Omicron variant, nearly 5˜10-fold (FIGS. 12I-12J). However, the 3^{rd} booster of the circRNA^{RBD-Delta} or circRNA^{RBD-Omicron} vaccine failed to enhance the neutralizing capability against Omicron variant, and the 3^{rd} booster of the circRNA^{RBD-Omicron} vaccine even sharply reduced the capability against Omicron variant (FIGS. 12I-12J).

Besides, mice immunized with 2 doses of circRNA^{RBD} vaccines received 3^{rd} booster of circRNA^{RBD} or circRNA^{RBD-Delta}, vaccines at 8 weeks post the 2^{nd} dose (FIG. 12K). The neutralizing antibodies against the Omicron variant pseudovirus were also detected at 1 week post the 3^{rd} boost (FIG. 12K). The results showed that the 3^{rd} booster of both the same circRNA^{RBD} vaccine or circRNA^{RBD-Delta}, a vaccine dramatically boosted the neutralizing capability against Delta and Omicron variant, especially the 3^{rd} booster of circRNA^{RBD-Delta} vaccine, which elevated the NT50 value from 6×10² to 3.8×10⁴ against Omicron variant (FIGS. 12L-12M).

Altogether, the above results indicated that not only the two doses of de novo circRNA^{RBD-Omicron} vaccines were a coping strategy for the emerging SARS-CoV-2 Omicron variant, but also the 3^{rd} booster of circRNA^{RBD-Delta} vaccine after two-dose immunization might be an effective solution to the current epidemic of SARS-CoV-2 Delta and Omicron variants.

### Example 17: circRNA Vaccines

COVID-19 is still a fast-growing global health crisis with circulating SAS-CoV-2 variants evading current vaccines elicited antibodies^{53,65,66} Examples 1-13 demonstrate the effectiveness of using circRNA to produce SARS-CoV-2 vaccines.

Several studies have reported that the full-length Spike protein (mRNA-1273 and BNT162b2)^{21,22,27} or RBD-based mRNA vaccines elicit neutralizing antibodies and cellular immune responses^{23,26,67}. As reported, most effective neutralizing antibodies recognize the RBD region of S protein^{62,63,68-71}, and targeting RBD may induce less amount of non-neutralizing antibodies^{23-26,67}. Given that RBD trimers were similar to the trimeric ectodomain of full-length Spike proteins⁴⁸ and were superior in binding hACE2 compared to their monomeric counterparts⁴⁸, circRNA was chosen as the immunogen to express RBD trimers. The RBD trimmers were functional (FIG. 1I) and indeed induced potent and sustained neutralizing antibodies against authentic SARS-CoV-2 virus (FIGS. 2A-2O and FIGS. 5A-5J).

CircRNA^{RBD-501Y,V2} immunized; mice produced high titers of neutralizing antibodies. Given that K417N-E484K-N501Y mutant in RBD reduces its interactions with certain neutralizing antibodies^{66,72}, it is demonstrated that neutralizing antibodies produced by mice immunized with circRNA^{RBD} or circRNA^{RBD-Beta} had preferential neutralizing abilities to their corresponding virus strains. Recent studies suggested that Beta variant showed no higher infectivity but had immune escape capability⁵³⁻⁵⁸, and varieties of vaccines were reported to be less effective against SARS-CoV-2 variants^{65,73-77}. There have also been reported vaccine breakthrough infections with SARS-CoV-2 variants⁷⁸. Thus, it is imminent to develop and implement vaccines against emerging variants, and the cricRNA vaccine is such a platform that could be rapidly tailored for specific variants. For example, a vaccine contains E484K, N501Y, and L452R mutations in the RBD can be developed quickly via the circRNA platform to deal with a potential outbreak caused by the SARS-CoV-2 variants.

This generalizable strategy for designing immunogens is highlighted in the present disclosure. The coding sequence of circular RNA can be quickly adapted to deal with any emerging SARS-CoV-2 variants of concern, including Alpha, Beta, Gamma, Oelta and Omicron^{66,85,86}. Moreover, circular RNAs could be quickly generated in large quantities in vitro, and they do not require any nucleotide modification, strikingly different from the canonical mRNA vaccines. Because of the covalently closed ring structure, the circRNA holds inherent stability.⁶¹. Our data demonstrated that both the circRNA^{RBD} and encapsulated circRNA^{RBD}-LNP complex were highly thermostable at 4° C. for about 4 weeks (FIG. 4E).

### Example 18. Luciferase Expression by Naked circRNA^{Luc} in Mice

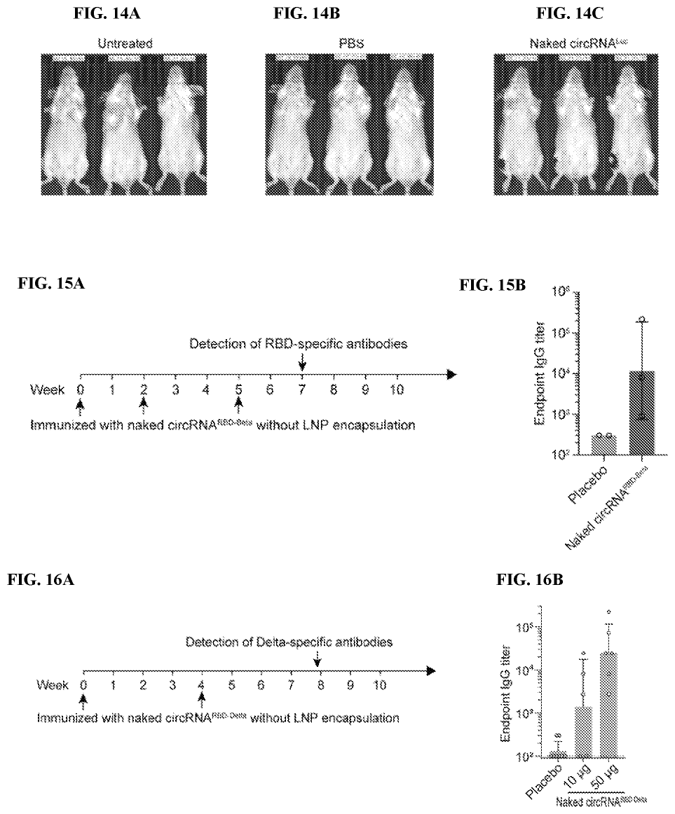

Group I ribozyme autocatalysis strategies were adopted to generate circRNA^{Luc} encoding firefly luciferase (Luc). The circRNA^{Luc} was then diluted in PBS. Mice were injected intramuscularly with PBS or 20 μg of naked circRNA^{Luc}, while untreated group served as mock control. 24 hr post injection, mice were injected intraperitoneally with luciferin and transferred to the IVIS imaging system. Naked circRNA^{Luc} showed local expression of firefly luciferase in the injection site (FIGS. 14A-14C).

### Example 19. CircRNA Vaccines Comprising Naked circRNAs Elicited SARS-CoV-2 RBD-Specific Antibodies in Mice

Firstly, Group I ribozyme autocatalysis strategies were adopted to generate circRNA^{RBD-Beta} vaccine comprising naked circRNA^{RBD-Beta} encoding RBD antigens of Beta variant without LNP encapsulation. The circRNA^{RBD-Beta} was then diluted in PBS. Mice were immunized i.m. with 2 doses of 100 μg circRNA^{RBD-Beta} vaccine twice at a 2-week interval and a 3^{rd} dose of 250 μg circRNA^{RBD-Beta} vaccine at 3 weeks post boost, while PBS were used as the placebo control. 2 weeks post 3^{rd} boost, the serum samples from immunized mice were collected for the detection of RBD specific antibodies (FIG. 15A). CircRNA^{RBD-Beta} vaccines could induce RBD-specific IgG binding antibodies in mice (FIG. 15B).

Considering that RBD-Delta has more Immunogenicity and LNP-encapsulated circRNA^{RBD-Delta} vaccine could induce higher IgG titer in mice than LNP-encapsulated circRNA^{RBD-Beta} vaccine. Group I ribozyme autocatalysis strategies were adopted to generate circRNA^{RBD-Delta} vaccine comprising naked circRNA^{RBD-Delta} encoding RBD antigens of Delta variant without LNP encapsulation. The circRNA^{RBD-Delta} was then diluted in PBS. Mice were immunized i.m. with 10 or 50 μg of circRNA^{RBD-Delta} vaccine twice at a 4-week interval, while PBS were used as the placebo control. 25 days post boost, the serum samples from immunized mice were collected for the detection of RBD-Delta specific antibodies (FIG. 16A). CircRNA^{RBD-Delta} vaccines could induce RBD-Delta specific IgG binding antibodies in mice, while 50 μg group generated higher IgG level than 10 μg group (FIG. 16B).

### Example 20. Naked circRNA Vaccines Elicited Higher Antibodies than mRNA Vaccines in Mice

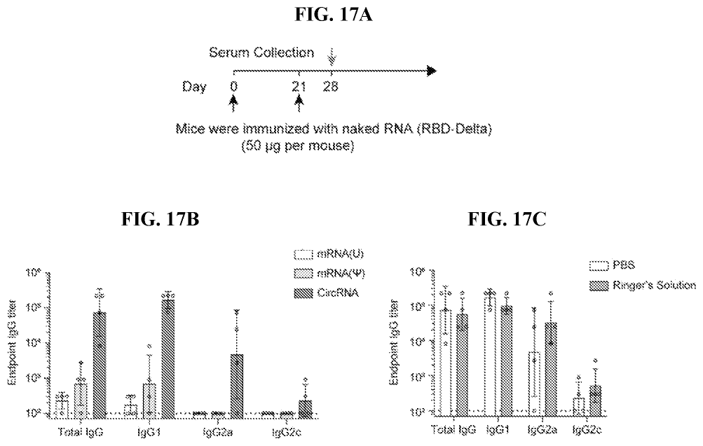

Group I ribozyme autocatalysis strategies were adopted to generate naked circRNA^{RBD-Delta} vaccine encoding RBD antigens of Delta variant without LNP encapsulation. For naked mRNA^{RBD-Delta} vaccines, modified or unmodified mRNA was generated with or without 1mΨ-modified UTP. RNA vaccines were then diluted in PBS. Mice were immunized 50 μg of naked RNA vaccines twice at a 3-week interval. 7 days post boost, the serum samples from immunized mice were collected for the detection of RBD-Delta specific antibodies, including total IgG, IgG1, IgG2a and IgG2c (FIG. 17A). Naked circRNA^{RBD-Delta} vaccines could induce potent antibodies in mice, while naked modified or unmodified mRNA^{RBD-Delta} vaccines could not (FIG. 17B). Besides, given that Ringer's solution was used in naked mRNA vaccines before, the difference between PBS and Ringer's solution as the buffer of naked circRNA was compared. Using Ringer's solution, circRNA vaccine could induce higher IgG2a and IgG2c, which suggests higher quality of antibodies (FIG. 17C).

### Example 21. Optimization of circRNA Vaccine Encoding Omicron BA.1 RBD Antigen

Group I ribozyme autocatalysis strategies were adopted to generate circRNA^{RBD-Omicron} vaccine encoding RBD antigens of Omicron variant before. It showed low efficiency against naïve or Delta variant but remained some possibility against new emerging omicron and its sublineage variants. Here, some mutations were rationally introduced and the length of RBD antigen was modified, trying to improve the performance of the omicron BA.1 vaccine. Besides, the benefit of trimerization domain folden was also tested. All antigens were expressed by circRNA generated by group I intron autocatalysis strategy.

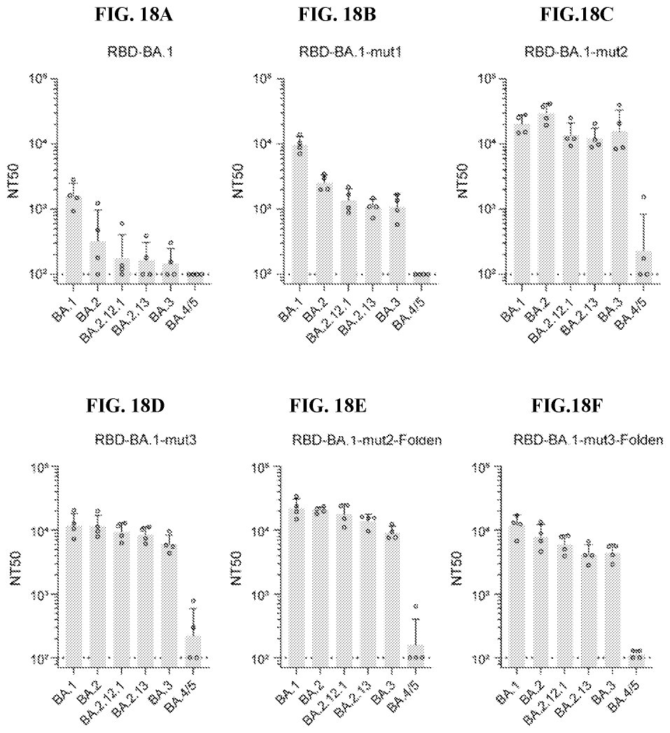

Mice were immunized 5 μg of LNP-circRNA vaccines twice at a 2-week interval. 7 days post boost, the serum samples from immunized mice were collected for the detection of neutralizing antibodies against multiple SARS-CoV-2 variants by pseudovirus neutralization assay. For the original Omicron BA.1 vaccaine, it shows low efficiency against omicron and some omicron sublineage. (FIG. 18A). After optimization, all new vaccines showed better performance, but could not effectively protect against BA.4/5 variant (FIGS. 18B-F).

The detailed antigen information of each vaccine is shown below:
RBD-BA.1: RBD (319-541)-Omicron BA.1
RBD-BA.1-mut1: RBD (319-541)-Omicron BA.1+S375A.
RBD-BA.1-mut2: RBD (319-537)-Omicron BA.1+S375A.
RBD-BA.1-mut3: RBD (319-537)-Omicron BA.1+S375A+L452R

### Example 22. CircRNA^{RBD-BA.4/5} vaccine induces potent neutralizing antibodies against some variants of Omicron and its sublineage

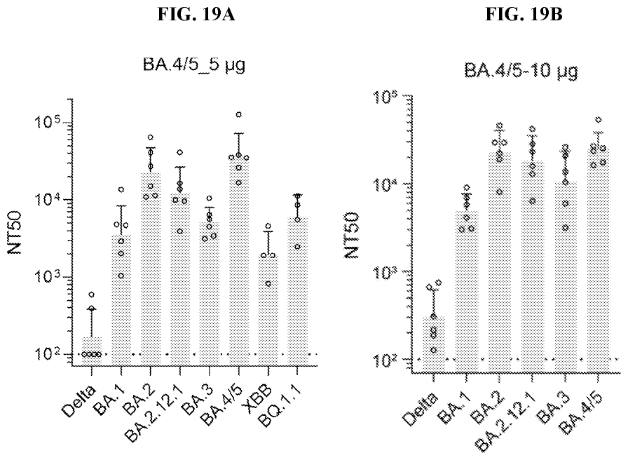

Group I ribozyme autocatalysis strategies were adopted to generate circRNA^{RBD-BA.4/5} vaccine encoding RBD antigens of Omicron BA.4/5. Mice were immunized 5 μg or 10 μg of LNP-circRNA vaccines twice at a 3-week interval. 7 days post boost, the serum samples from immunized mice were collected for the detection of neutralizing antibodies against multiple SARS-CoV-2 variants by pseudovirus neutralization assay. The sera from vaccinated mice effectively neutralized the pseudovirus in a dose-dependent manner and showed preferential neutralizing activity against BA.4/5 variant (FIG. 19A-B).

### Example 23. CircRNA^{RBD-Trimer} Vaccine Induces Potent Neutralizing Antibodies Against Some Variants of Omicron and its Sublineage

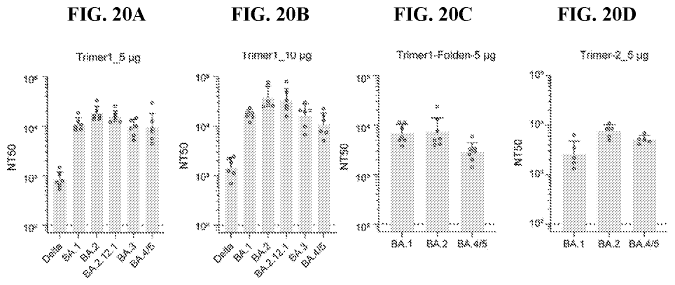

To generate broad-spectrum SARS-CoV-2 vaccine, the strategy of tandem expressing multiple domains from distinct variants was applied. For proof of concept, two trimer vaccine encoding three domains of three variants was generated. Group I ribozyme autocatalysis strategies were adopted to generate circRNA^{RBD-Trimer} vaccine. Mice were immunized 5 μg or 10 μg of LNP-circRNA vaccines twice at a 2-week interval. 7 days post boost, the serum samples from immunized mice were collected for the detection of neutralizing antibodies against multiple SARS-CoV-2 variants by pseudovirus neutralization assay. The sera from vaccinated mice effectively neutralized the pseudovirus and showed some broad-spectrum neutralizing activity (FIG. 20A-D).

The detailed antigen information of each vaccine is shown below:
Trimer1: RBD^{Delta}-RBD^{BA.1}-RBD^{BA.4/5}
Trimer2: NTD^{BA.4/5}-RBD^{BA.4/5}-RBD^{Delta}

The examples set forth above are provided to give those of ordinary skill in the art a complete disclosure and description of how to make and use the embodiments of the circRNA, vaccines, compositions, and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of the artisans to which the invention pertains.

Many modifications and variations of this application can be made without departing from its spirit and scope, as will be apparent to those skilled in the art. The specific embodiments and examples described herein are offered by way of example only.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

### REFERENCES

1 Wu, F. et al. A new coronavirus associated with human respiratory disease in China. Nature 579, 265-269, doi:10.1038/s41586-020-2008-3 (2020).
2 Zhou, P. et al. A pneumonia outbreak associated with a new coronavirus of probable bat origin. Nature 579, 270-273, doi:10.1038/s41586-020-2012-7 (2020).
3 V'Kovski, P., Kratzel, A., Steiner, S., Stalder, H. & Thiel, V. Coronavirus biology and replication: implications for SARS-CoV-2. Nat Rev Microbiol 19, 155-170, doi:10.1038/s41579-020-00468-6 (2021).
4 Kim, D. et al. The Architecture of SARS-CoV-2 Transcriptome. Cell 181, 914-921 e910, doi:10.1016/j.cell.2020.04.011 (2020).
5 Hoffmann, M. et al. SARS-CoV-2 Cell Entry Depends on ACE2 and TMPRSS2 and Is Blocked by a Clinically Proven Protease Inhibitor. Cell 181, 271-280 e278, doi:10.1016/j.cell.2020.02.052 (2020).
6 Yan, R. et al. Structural basis for the recognition of SARS-CoV-2 by full-length human ACE2. Science 367, 1444-1448, doi:10.1126/science.abb2762 (2020).
7 Wrapp, D. et al. Cryo-EM structure of the 2019-nCOV spike in the prefusion conformation. Science 367, 1260-1263, doi:10.1126/science.abb2507 (2020).
8 Shang, J. et al. Cell entry mechanisms of SARS-CoV-2. Proc Natl Acad Sci USA 117, 11727-11734, doi:10.1073/pnas.2003138117 (2020).
9 Walls, A. C. et al. Structure, Function, and Antigenicity of the SARS-CoV-2 Spike Glycoprotein. Cell 181, 281-292 e286, doi:10.1016/j.cell.2020.02.058 (2020).
10 Hsieh, C. L. et al. Structure-based design of prefusion-stabilized SARS-CoV-2 spikes. Science 369, 1501-1505, doi:10.1126/science.abd0826 (2020).
11 Bangaru, S. et al. Structural analysis of full-length SARS-CoV-2 spike protein from an advanced vaccine candidate. Science 370, 1089-1094, doi:10.1126/science.abe1502 (2020).
12 Krammer, F. SARS-CoV-2 vaccines in development. Nature 586, 516-527, doi:10.1038/s41586-020-2798-3 (2020).
13 Mullard, A. COVID-19 vaccine development pipeline gears up. Lancet 395, 1751-1752, doi:10.1016/S0140-6736(20)31252-6 (2020).
14 Gao, Q. et al. Development of an inactivated vaccine candidate for SARS-CoV-2. Science 369, 77-81, doi:10.1126/science.abc1932 (2020).
15 van Doremalen, N. et al. ChAdOx1 nCOV-19 vaccine prevents SARS-CoV-2 pneumonia in rhesus macaques. Nature 586, 578-582, doi:10.1038/s41586-020-2608-y (2020).
16 Sanchez-Felipe, L. et al. A single-dose live-attenuated YF17D-vectored SARS-CoV-2 vaccine candidate. Nature 590, 320-325, doi:10.1038/s41586-020-3035-9 (2021).
17 Zhu, F. C. et al. Safety, tolerability, and immunogenicity of a recombinant adenovirus type-5 vectored COVID-19 vaccine: a dose-escalation, open-label, non-randomised, first-in-human trial. Lancet 395, 1845-1854, doi:10.1016/S0140-6736(20)31208-3 (2020).
18 Yang, J. et al. A vaccine targeting the RBD of the S protein of SARS-CoV-2 induces protective immunity. Nature 586, 572-577, doi:10.1038/s41586-020-2599-8 (2020).
19 Dai, L. et al. A Universal Design of Betacoronavirus Vaccines against COVID-19, MERS, and SARS. Cell 182, 722-733 e711, doi:10.1016/j.cell.2020.06.035 (2020).
20 Yu, J. et al. DNA vaccine protection against SARS-CoV-2 in rhesus macaques. Science 369, 806-811, doi:10.1126/science.abc6284 (2020).
21 Corbett, K. S. et al. SARS-CoV-2 mRNA vaccine design enabled by prototype pathogen preparedness. Nature 586, 567-571, doi:10.1038/s41586-020-2622-0 (2020).
22 Corbett, K. S. et al. Evaluation of the mRNA-1273 Vaccine against SARS-CoV-2 in Nonhuman Primates. N Engl J Med 383, 1544-1555, doi:10.1056/NEJMoa2024671 (2020).
23 Zhang, N. N. et al. A Thermostable mRNA Vaccine against COVID-19. Cell 182, 1271-1283 e1216, doi:10.1016/j.cell.2020.07.024 (2020).
24 Laczko, D. et al. A Single Immunization with Nucleoside-Modified mRNA Vaccines Elicits Strong Cellular and Humoral Immune Responses against SARS-CoV-2 in Mice. Immunity 53, 724-732 e727, doi:10.1016/j.immuni.2020.07.019 (2020).
25 Huang, Q. et al. A single-dose mRNA vaccine provides a long-term protection for hACE2 transgenic mice from SARS-CoV-2. Nat Commun 12, 776, doi:10.1038/s41467-021-21037-2 (2021).
26 Sahin, U. et al. COVID-19 vaccine BNT162b1 elicits human antibody and THI T cell responses. Nature 586, 594-599, doi:10.1038/s41586-020-2814-7 (2020).
27 Vogel, A. B. et al. BNT162b vaccines protect rhesus macaques from SARS-CoV-2. Nature, doi:10.1038/s41586-021-03275-y (2021).
28 Pardi, N., Hogan, M. J., Porter, F. W. & Weissman, D. mRNA vaccines—a new era in vaccinology. Nat Rev Drug Discov 17, 261-279, doi:10.1038/nrd.2017.243 (2018).
29 Jackson, N. A. C., Kester, K. E., Casimiro, D., Gurunathan, S. & DeRosa, F. The promise of mRNA vaccines: a biotech and industrial perspective. NPJ Vaccines 5, 11, doi:10.1038/s41541-020-0159-8 (2020).
30 Durymanov, M. & Reineke, J. Non-viral Delivery of Nucleic Acids: Insight Into Mechanisms of Overcoming Intracellular Barriers. Front Pharmacol 9, 971, doi:10.3389/fphar.2018.00971 (2018).
31 Fenton, O. S. et al. Bioinspired Alkenyl Amino Alcohol Ionizable Lipid Materials for Highly Potent In Vivo mRNA Delivery. Adv Mater 28, 2939-2943, doi:10.1002/adma.201505822 (2016).
32 Wesselhoeft, R. A. et al. RNA Circularization Diminishes Immunogenicity and Can Extend Translation Duration In Vivo. Mol Cell 74, 508-520 e504, doi:10.1016/j.molcel.2019.02.015 (2019).
33 Chen, Y. G. et al. N6-Methyladenosine Modification Controls Circular RNA Immunity. Mol Cell 76, 96-109 e109, doi:10.1016/j.molcel.2019.07.016 (2019).
34 Zhang, X. O. et al. Complementary sequence-mediated exon circularization. Cell 159, 134-147, doi:10.1016/j.cell.2014.09.001 (2014).
35 Chen, L. L. The biogenesis and emerging roles of circular RNAs. Nat Rev Mol Cell Biol 17, 205-211, doi:10.1038/nrm.2015.32 (2016).
36 Kristensen, L. S. et al. The biogenesis, biology and characterization of circular RNAs. Nat Rev Genet 20, 675-691, doi:10.1038/s41576-019-0158-7 (2019).
37 Memczak, S. et al. Circular RNAs are a large class of animal RNAs with regulatory potency. Nature 495, 333-338, doi:10.1038/nature11928 (2013).
38 Enuka, Y. et al. Circular RNAs are long-lived and display only minimal early alterations in response to a growth factor. Nucleic Acids Res 44, 1370-1383, doi:10.1093/nar/gkv1367 (2016).
39 Legnini, I. et al. Circ-ZNF609 Is a Circular RNA that Can Be Translated and Functions in Myogenesis. Mol Cell 66, 22-37 e29, doi:10.1016/j.molcel.2017.02.017 (2017).
40 Zhang, M. et al. A novel protein encoded by the circular form of the SHPRH gene suppresses glioma tumorigenesis. Oncogene 37, 1805-1814, doi:10.1038/s41388-017-0019-9 (2018).
41 Zhang, M. et al. A peptide encoded by circular form of LINC-PINT suppresses oncogenic transcriptional elongation in glioblastoma. Nat Commun 9, 4475, doi:10.1038/s41467-018-06862-2 (2018).
42 Gao, X. et al. Circular RNA-encoded oncogenic E-cadherin variant promotes glioblastoma tumorigenicity through activation of EGFR-STAT3 signalling. Nat Cell Biol, doi:10.1038/s41556-021-00639-4 (2021).
43 Wesselhoeft, R. A., Kowalski, P. S. & Anderson, D. G. Engineering circular RNA for potent and stable translation in eukaryotic cells. Nat Commun 9, 2629, doi:10.1038/s41467-018-05096-6 (2018).
44 Yang, Y. et al. Extensive translation of circular RNAs driven by N(6)-methyladenosine. Cell Res 27, 626-641, doi:10.1038/cr.2017.31 (2017).
45 Kou, Y. et al. Tissue plasminogen activator (tPA) signal sequence enhances immunogenicity of MVA-based vaccine against tuberculosis. Immunol Lett 190, 51-57, doi:10.1016/j.imlet.2017.07.007 (2017).
46 Richner, J. M. et al. Modified mRNA Vaccines Protect against Zika Virus Infection. Cell 169, 176, doi:10.1016/j.cell.2017.03.016 (2017).
47 Pardi, N. et al. Zika virus protection by a single low-dose nucleoside-modified mRNA vaccination. Nature 543, 248-251, doi:10.1038/nature21428 (2017).
48 Bouwman, K. M. et al. Multimerization- and glycosylation-dependent receptor binding of SARS-CoV-2 spike proteins. PLOS Pathog 17, e1009282, doi:10.1371/journal.ppat. 1009282 (2021).
49 Papanikolopoulou, K., van Raaij, M. J. & Mitraki, A. Creation of hybrid nanorods from sequences of natural trimeric fibrous proteins using the fibritin trimerization motif. Methods Mol Biol 474, 15-33, doi:10.1007/978-1-59745-480-3_2 (2008).
50 Ou, X. et al. Characterization of spike glycoprotein of SARS-CoV-2 on virus entry and its immune cross-reactivity with SARS-CoV. Nat Commun 11, 1620, doi:10.1038/s41467-020-15562-9 (2020).
51 Tan, C. W. et al. A SARS-CoV-2 surrogate virus neutralization test based on antibody-mediated blockage of ACE2-spike protein-protein interaction. Nat Biotechnol 38, 1073-1078, doi:10.1038/s41587-020-0631-z (2020).
52 Sette, A. & Crotty, S. Adaptive immunity to SARS-CoV-2 and COVID-19. Cell 184, 861-880, doi:10.1016/j.cell.2021.01.007 (2021).
53 Wang, P. et al. Antibody Resistance of SARS-CoV-2 Variants B.1.351 and B.1.1.7. Nature, doi:10.1038/s41586-021-03398-2 (2021).
54 Li, Q. et al. SARS-CoV-2 501Y.V2 variants lack higher infectivity but do have immune escape. Cell 184, 2362-2371 e2369, doi:10.1016/j.cell.2021.02.042 (2021).
55 Chen, R. E. et al. Resistance of SARS-CoV-2 variants to neutralization by monoclonal and serum-derived polyclonal antibodies. Nat Med 27, 717-726, doi:10.1038/s41591-021-01294-w (2021).
56 Planas, D. et al. Sensitivity of infectious SARS-CoV-2 B.1.1.7 and B.1.351 variants to neutralizing antibodies. Nat Med, doi:10.1038/s41591-021-01318-5 (2021).
57 Hoffmann, M. et al. SARS-CoV-2 variants B.1.351 and P.1 escape from neutralizing antibodies. Cell 184, 2384-2393 e2312, doi:10.1016/j.cell.2021.03.036 (2021).
58 Cele, S. et al. Escape of SARS-CoV-2 501Y.V2 from neutralization by convalescent plasma. Nature, doi:10.1038/s41586-021-03471-w (2021).
59 Montagutelli, X. et al., doi:10.1101/2021.03.18.436013 (2021).
60 Gu, H. et al. Adaptation of SARS-CoV-2 in BALB/c mice for testing vaccine efficacy. Science 369, 1603-1607, doi:10.1126/science.abc4730 (2020).
61 Fischer, J. W. & Leung, A. K. CircRNAs: a regulator of cellular stress. Crit Rev Biochem Mol Biol 52, 220-233, doi:10.1080/10409238.2016.1276882 (2017).
62 Xiang, Y. et al. Versatile and multivalent nanobodies efficiently neutralize SARS-CoV-2. Science 370, 1479-1484, doi:10.1126/science.abe4747 (2020).
63 Schoof, M. et al. An ultrapotent synthetic nanobody neutralizes SARS-CoV-2 by stabilizing inactive Spike. Science 370, 1473-1479, doi:10.1126/science.abe3255 (2020).
64 Linsky, T. W. et al. De novo design of potent and resilient hACE2 decoys to neutralize SARS-CoV-2. Science 370, 1208-1214, doi:10.1126/science.abe0075 (2020).
65 Muik, A. et al. Neutralization of SARS-CoV-2 lineage B.1.1.7 pseudovirus by BNT162b2 vaccine-elicited human sera. Science, doi:10.1126/science.abg6105 (2021).
66 Wang, Z. et al. mRNA vaccine-elicited antibodies to SARS-CoV-2 and circulating variants. Nature, doi:10.1038/s41586-021-03324-6 (2021).
67 Tai, W. et al. A novel receptor-binding domain (RBD)-based mRNA vaccine against SARS-CoV-2. Cell Res 30, 932-935, doi:10.1038/s41422-020-0387-5 (2020).
68 Cao, Y. et al. Potent Neutralizing Antibodies against SARS-CoV-2 Identified by High-Throughput Single-Cell Sequencing of Convalescent Patients' B Cells. Cell 182, 73-84 e16, doi:10.1016/j.cell.2020.05.025 (2020).
69 Du, S. et al. Structurally Resolved SARS-CoV-2 Antibody Shows High Efficacy in Severely Infected Hamsters and Provides a Potent Cocktail Pairing Strategy. Cell 183, 1013-1023 e1013, doi:10.1016/j.cell.2020.09.035 (2020).
70 Barnes, C. O. et al. SARS-CoV-2 neutralizing antibody structures inform therapeutic strategies. Nature 588, 682-687, doi:10.1038/s41586-020-2852-1 (2020).
71 Koenig, P. A. et al. Structure-guided multivalent nanobodies block SARS-CoV-2 infection and suppress mutational escape. Science 371, doi:10.1126/science.abe6230 (2021).
72 Wibmer, C. K. et al. SARS-CoV-2 501Y.V2 escapes neutralization by South African COVID-19 donor plasma. Nat Med, doi:10.1038/s41591-021-01285-x (2021).
73 Garcia-Beltran, W. F. et al. Multiple SARS-CoV-2 variants escape neutralization by vaccine-induced humoral immunity. Cell 184, 2523, doi:10.1016/j.cell.2021.04.006 (2021).
74 Focosi, D. & Maggi, F. Neutralising antibody escape of SARS-CoV-2 spike protein: Risk assessment for antibody-based Covid-19 therapeutics and vaccines. Rev Med Virol, doi:10.1002/rmv.2231 (2021).
75 Gupta, R. K. Will SARS-CoV-2 variants of concern affect the promise of vaccines? Nat Rev Immunol, doi:10.1038/s41577-021-00556-5 (2021).
76 Madhi, S. A. et al. Efficacy of the ChAdOx1 nCOV-19 Covid-19 Vaccine against the B.1.351 Variant. N Engl J Med, doi:10.1056/NEJMoa2102214 (2021).
77 Wang, G. L. et al. Susceptibility of Circulating SARS-CoV-2 Variants to Neutralization. N Engl J Med, doi:10.1056/NEJMc2103022 (2021).
78 Hacisuleyman, E. et al. Vaccine Breakthrough Infections with SARS-CoV-2 Variants. N Engl J Med, doi:10.1056/NEJMoa2105000 (2021).
79 Chan, K. K. et al. Engineering human ACE2 to optimize binding to the spike protein of SARS coronavirus 2. Science 369, 1261-1265, doi:10.1126/science.abc0870 (2020).
80 Glasgow, A. et al. Engineered ACE2 receptor traps potently neutralize SARS-CoV-2. Proc Natl Acad Sci USA 117, 28046-28055, doi:10.1073/pnas.2016093117 (2020).
81 Boutros, C. et al. Safety profiles of anti-CTLA-4 and anti-PD-1 antibodies alone and in combination. Nat Rev Clin Oncol 13, 473-486, doi:10.1038/nrclinonc.2016.58 (2016).
82 He, X. & Xu, C. Immune checkpoint signaling and cancer immunotherapy. Cell Res 30, 660-669, doi:10.1038/s41422-020-0343-4 (2020).
83 Sabapathy, K. & Lane, D. P. Therapeutic targeting of p53: all mutants are equal, but some mutants are more equal than others. Nat Rev Clin Oncol 15, 13-30, doi:10.1038/nrclinonc.2017.151 (2018).
84 Mukhopadhyay, S., Vander Heiden, M. G. & McCormick, F. The Metabolic Landscape of RAS-Driven Cancers from biology to therapy. Nat Cancer 2, 271-283, doi:10.1038/s43018-021-00184-x (2021).
85 Davies, N. G. et al. Increased hazard of death in community-tested cases of SARS-CoV-2 Variant of Concern 202012/01. medRxiv, doi:10.1101/2021.02.01.21250959 (2021).
86 Lauring, A. S. & Hodcroft, E. B. Genetic Variants of SARS-CoV-2-What Do They Mean? JAMA 325, 529-531, doi:10.1001/jama.2020.27124 (2021).
87 Ickenstein, L. M. & Garidel, P. Lipid-based nanoparticle formulations for small molecules and RNA drugs. Expert Opin Drug Deliv 16, 1205-1226, doi:10.1080/17425247.2019.1669558 (2019).
88 Pinto, D. et al. Cross-neutralization of SARS-CoV-2 by a human monoclonal SARS-CoV antibody. Nature 583, 290-295, doi:10.1038/s41586-020-2349-y (2020).

EXEMPLARY SEQUENCES
Full-length S protein sequence of SARS-CoV-2
SEQ ID NO: 1
MFVFLVLLPLVSSQCVNLTTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLMDLE
GKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSETK
CTLKSFTVEKGIYQTSNFRVQPTESIVRFPNITNLCPFGEVENATRFASVYAWNRKRISN
CVADYSVLYNSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTEIYQAGSTPC
NGVEGFNCYFPLQSYGFQPTNGVGYQPYRVVVLSFELLHAPATVCGPKKSTNLVKNKCVN
FNFNGLTGTGVLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCLIGAEHVNNSY
ECDIPIGAGICASYQTQTNSPRRARSVASQSIIAYTMSLGAENSVAYSNNSIAIPTNFTI
SVTTEILPVSMTKTSVDCTMYICGDSTECSNLLLQYGSFCTQLNRALTGIAVEQDKNTQE
VFAQVKQIYKTPPIKDFGGFNFSQILPDPSKPSKRSFIEDLLFNKVTLADAGFIKQYGDC
LGDIAARDLICAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQDVVNQNAQALN
TLVKQLSSNFGAISSVLNDILSRLDKVEAEVQIDRLITGRLQSLQTYVTQQLIRAAEIRA
SANLAATKMSECVLGQSKRVDFCGKGYHLMSFPQSAPHGVVFLHVTYVPAQEKNFTTAPA
ICHDGKAHFPREGVFVSNGTHWFVTQRNFYEPQIITTDNTFVSGNCDVVIGIVNNTVYDP
LQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKFDEDD
SEPVLKGVKLHYT
RBD amino acid residues 319-542 of S protein
SEQ ID NO: 2
RVQPTESIVRFPNITNLCPFGEVFNATRFASVYAWNRKRISNCVADYSVLYNSASFSTFK
CYGVSPTKLNDLCFTNVYADSFVIRGDEVRQIAPGQTGKIADYNYKLPDDFTGCVIAWNS
NNLDSKVGGNYNYLYRLFRKSNLKPFERDISTEIYQAGSTPCNGVEGFNCYFPLQSYGFQ
PTNGVGYQPYRVVVLSFELLHAPATVCGPKKSTNLVKNKCVNF
C-terminal Foldon domain of a T4 fibritin domain
SEQ ID NO: 3
GSGYIPEAPRDGQAYVRKDGEWVLLSTFLGRS
GCN4-based leucine zipper domain
SEQ ID NO: 4
RMKQIEDKIEEILSKIYHIENEIARIKKLIGER
Exemplary peptide linker
SEQ ID NO: 5
GGGGSGGGGS
wildtype S2 region of SARS-CoV-2 S protein
SEQ ID NO: 6
SVASQSIIAYTMSLGAENSVAYSNNSIAIPTNFTISVTTEILPVSMTKTSVDCTMYICGD
STECSNLLLQYGSFCTQLNRALTGIAVEQDKNTQEVFAQVKQIYKTPPIKDFGGFNFSQI
LPDPSKPSKRSFIEDLLFNKVTLADAGFIKQYGDCLGDIAARDLICAQKFNGLTVLPPLL
TDEMIAQYTSALLAGTITSGWTFGAGAALQIPFAMQMAYRFNGIGVTQNVLYENQKLIAN
QFNSAIGKIQDSLSSTASALGKLQDVVNQNAQALNTLVKQLSSNFGAISSVLNDILSRLD
KVEAEVQIDRLITGRLQSLQTYVTQQLIRAAEIRASANLAATKMSECVLGQSKRVDFCGK
GYHLMSFPQSAPHGVVFLHVTYVPAQEKNFTTAPAICHDGKAHFPREGVFVSNGTHWFVT
QRNFYEPQIITTDNTFVSGNCDVVIGIVNNTVYDPLQPELDSFKEELDKYFKNHTSPDVD
LGDISGINASVVNIQKEIDRLNEVAKNLNESLIDLQELGKYEQYIKWPWYIWLGFIAGLI
AIVMVTIMLCCMTSCCSCLKGCCSCGSCCKFDEDDSEPVLKGVKLHYT
K986P/V987P S2 region sequence of SARS-CoV-2 S protein
SEQ ID NO: 7
SVASQSIIAYTMSLGAENSVAYSNNSIAIPTNFTISVTTEILPVSMTKTSVDCTMYICGD
STECSNLLLQYGSFCTQLNRALTGIAVEQDKNTQEVFAQVKQIYKTPPIKDFGGFNFSQI
LPDPSKPSKRSFIEDLLFNKVTLADAGFIKQYGDCLGDIAARDLICAQKFNGLTVLPPLL
TDEMIAQYTSALLAGTITSGWTFGAGAALQIPFAMQMAYRFNGIGVTQNVLYENQKLIAN
QFNSAIGKIQDSLSSTASALGKLQDVVNQNAQALNTLVKQLSSNFGAISSVLNDILSRLD
PPEAEVQIDRLITGRLQSLQTYVTQQLIRAAEIRASANLAATKMSECVLGQSKRVDFCGK
GYHLMSFPQSAPHGVVFLHVTYVPAQEKNFTTAPAICHDGKAHFPREGVFVSNGTHWFVT
QRNFYEPQIITTDNTFVSGNCDVVIGIVNNTVYDPLQPELDSFKEELDKYFKNHTSPDVD
LGDISGINASVVNIQKEIDRLNEVAKNLNESLIDLQELGKYEQYIKWPWYIWLGFIAGLI
AIVMVTIMLCCMTSCCSCLKGCCSCGSCCKFDEDDSEPVLKGVKLHYT
wildtype amino acid residues 2-1273 sequence of S protein
of SARS-CoV-2
SEQ ID NO: 8
FVFLVLLPLVSSQCVNLTTRTQLPPAYTNSFTRGVYYPDKVFRSSVLHSTQDLFLPFFSN
VTWFHAIHVSGTNGTKRFDNPVLPFNDGVYFASTEKSNIIRGWIFGTTLDSKTQSLLIVN
NATNVVIKVCEFQFCNDPFLGVYYHKNNKSWMESEFRVYSSANNCTFEYVSQPFLMDLEG
KQGNFKNLREFVFKNIDGYFKIYSKHTPINLVRDLPQGFSALEPLVDLPIGINITRFQTL
LALHRSYLTPGDSSSGWTAGAAAYYVGYLQPRTFLLKYNENGTITDAVDCALDPLSETKC
TLKSFTVEKGIYQTSNFRVQPTESIVRFPNITNLCPFGEVENATRFASVYAWNRKRISNC
VADYSVLYNSASFSTFKCYGVSPTKLNDLCFTNVYADSFVIRGDEVRQIAPGQTGKIADY
NYKLPDDFTGCVIAWNSNNLDSKVGGNYNYLYRLFRKSNLKPFERDISTEIYQAGSTPCN
GVEGFNCYFPLQSYGFQPTNGVGYQPYRVVVLSFELLHAPATVCGPKKSTNLVKNKCVNF
NFNGLTGTGVLTESNKKFLPFQQFGRDIADTTDAVRDPQTLEILDITPCSFGGVSVITPG
TNTSNQVAVLYQDVNCTEVPVAIHADQLTPTWRVYSTGSNVFQTRAGCLIGAEHVNNSYE
CDIPIGAGICASYQTQTNSPRRARSVASQSIIAYTMSLGAENSVAYSNNSIAIPTNFTIS
VTTEILPVSMTKTSVDCTMYICGDSTECSNLLLQYGSFCTQLNRALTGIAVEQDKNTQEV
FAQVKQIYKTPPIKDFGGFNFSQILPDPSKPSKRSFIEDLLFNKVTLADAGFIKQYGDCL
GDIAARDLICAQKFNGLTVLPPLLTDEMIAQYTSALLAGTITSGWTFGAGAALQIPFAMQ
MAYRFNGIGVTQNVLYENQKLIANQFNSAIGKIQDSLSSTASALGKLQDVVNQNAQALNT
LVKQLSSNFGAISSVLNDILSRLDKVEAEVQIDRLITGRLQSLQTYVTQQLIRAAEIRAS
ANLAATKMSECVLGQSKRVDFCGKGYHLMSFPQSAPHGVVFLHVTYVPAQEKNFTTAPAI
CHDGKAHFPREGVFVSNGTHWFVTQRNFYEPQIITTDNTFVSGNCDVVIGIVNNTVYDPL
QPELDSFKEELDKYFKNHTSPDVDLGDISGINASVVNIQKEIDRLNEVAKNLNESLIDLQ
ELGKYEQYIKWPWYIWLGFIAGLIAIVMVTIMLCCMTSCCSCLKGCCSCGSCCKFDEDDS
EPVLKGVKLHYT
amino acid residues 2-1273 sequence of S protein of
SARS-CoV-2, Δ681-684
SEQ ID NO: 9
FVFLVLLPLVSSQCVNLTTRTQLPPAYTNSFTRGVYYPDKVFRSSVLHSTQDLFLPFFSN
VTWFHAIHVSGTNGTKRFDNPVLPFNDGVYFASTEKSNIIRGWIFGTTLDSKTQSLLIVN
NATNVVIKVCEFQFCNDPFLGVYYHKNNKSWMESEFRVYSSANNCTFEYVSQPFLMDLEG
KQGNFKNLREFVFKNIDGYFKIYSKHTPINLVRDLPQGFSALEPLVDLPIGINITRFQTL
LALHRSYLTPGDSSSGWTAGAAAYYVGYLQPRTFLLKYNENGTITDAVDCALDPLSETKC
TLKSFTVEKGIYQTSNFRVQPTESIVRFPNITNLCPFGEVENATRFASVYAWNRKRISNC
VADYSVLYNSASFSTFKCYGVSPTKLNDLCFTNVYADSFVIRGDEVRQIAPGQTGKIADY
NYKLPDDFTGCVIAWNSNNLDSKVGGNYNYLYRLFRKSNLKPFERDISTEIYQAGSTPCN
GVEGFNCYFPLQSYGFQPTNGVGYQPYRVVVLSFELLHAPATVCGPKKSTNLVKNKCVNF
NFNGLTGTGVLTESNKKFLPFQQFGRDIADTTDAVRDPQTLEILDITPCSFGGVSVITPG
TNTSNQVAVLYQDVNCTEVPVAIHADQLTPTWRVYSTGSNVFQTRAGCLIGAEHVNNSYE
CDIPIGAGICASYQTQTNSRSVASQSIIAYTMSLGAENSVAYSNNSIAIPTNFTISVTTE
ILPVSMTKTSVDCTMYICGDSTECSNLLLQYGSFCTQLNRALTGIAVEQDKNTQEVFAQV
KQIYKTPPIKDFGGFNFSQILPDPSKPSKRSFIEDLLFNKVTLADAGFIKQYGDCLGDIA
ARDLICAQKFNGLTVLPPLLTDEMIAQYTSALLAGTITSGWTFGAGAALQIPFAMQMAYR
FNGIGVTQNVLYENQKLIANQFNSAIGKIQDSLSSTASALGKLQDVVNQNAQALNTLVKQ
LSSNFGAISSVLNDILSRLDKVEAEVQIDRLITGRLQSLQTYVTQQLIRAAEIRASANLA
ATKMSECVLGQSKRVDFCGKGYHLMSFPQSAPHGVVFLHVTYVPAQEKNFTTAPAICHDG
KAHFPREGVFVSNGTHWFVTQRNFYEPQIITTDNTFVSGNCDVVIGIVNNTVYDPLQPEL
DSFKEELDKYFKNHTSPDVDLGDISGINASVVNIQKEIDRLNEVAKNLNESLIDLQELGK
YEQYIKWPWYIWLGFIAGLIAIVMVTIMLCCMTSCCSCLKGCCSCGSCCKFDEDDSEPVL
KGVKLHYT
amino acid residues 2-1273 sequence of S protein of
SARS-CoV-2, K986P V987P Δ681-684 sequence
SEQ ID NO: 10
FVFLVLLPLVSSQCVNLTTRTQLPPAYTNSFTRGVYYPDKVFRSSVLHSTQDLFLPFFSN
VTWFHAIHVSGTNGTKRFDNPVLPFNDGVYFASTEKSNIIRGWIFGTTLDSKTQSLLIVN
NATNVVIKVCEFQFCNDPFLGVYYHKNNKSWMESEFRVYSSANNCTFEYVSQPFLMDLEG
KQGNFKNLREFVFKNIDGYFKIYSKHTPINLVRDLPQGFSALEPLVDLPIGINITRFQTL
LALHRSYLTPGDSSSGWTAGAAAYYVGYLQPRTFLLKYNENGTITDAVDCALDPLSETKC
TLKSFTVEKGIYQTSNFRVQPTESIVRFPNITNLCPFGEVENATRFASVYAWNRKRISNC
VADYSVLYNSASFSTFKCYGVSPTKLNDLCFTNVYADSFVIRGDEVRQIAPGQTGKIADY
NYKLPDDFTGCVIAWNSNNLDSKVGGNYNYLYRLFRKSNLKPFERDISTEIYQAGSTPCN
GVEGENCYFPLQSYGFQPTNGVGYQPYRVVVLSFELLHAPATVCGPKKSTNLVKNKCVNF
NFNGLTGTGVLTESNKKFLPFQQFGRDIADTTDAVRDPQTLEILDITPCSFGGVSVITPG
TNTSNQVAVLYQDVNCTEVPVAIHADQLTPTWRVYSTGSNVFQTRAGCLIGAEHVNNSYE
CDIPIGAGICASYQTQTNSRSVASQSIIAYTMSLGAENSVAYSNNSIAIPTNFTISVTTE
ILPVSMTKTSVDCTMYICGDSTECSNLLLQYGSFCTQLNRALTGIAVEQDKNTQEVFAQV
KQIYKTPPIKDFGGFNFSQILPDPSKPSKRSFIEDLLFNKVTLADAGFIKQYGDCLGDIA
ARDLICAQKFNGLTVLPPLLTDEMIAQYTSALLAGTITSGWTFGAGAALQIPFAMQMAYR
FNGIGVTQNVLYENQKLIANQFNSAIGKIQDSLSSTASALGKLQDVVNQNAQALNTLVKQ
LSSNFGAISSVLNDILSRLDPPEAEVQIDRLITGRLQSLQTYVTQQLIRAAEIRASANLA
ATKMSECVLGQSKRVDFCGKGYHLMSFPQSAPHGVVFLHVTYVPAQEKNFTTAPAICHDG
KAHFPREGVFVSNGTHWFVTQRNFYEPQIITTDNTFVSGNCDVVIGIVNNTVYDPLQPEL
DSFKEELDKYFKNHTSPDVDLGDISGINASVVNIQKEIDRLNEVAKNLNESLIDLQELGK
YEQYIKWPWYIWLGFIAGLIAIVMVTIMLCCMTSCCSCLKGCCSCGSCCKFDEDDSEPVL
KGVKLHYT
Nucleic acid sequence of the wildtype S2 region sequence
SEQ ID NO: 11
AGTGTGGCTTCTCAAAGCATTATAGCATACACTATGTCTCTTGGTGCCGAAAATTCCGTG
GCCTATTCTAACAATTCAATCGCCATCCCAACCAACTTCACAATTAGCGTGACTACCGAA
ATACTGCCTGTGAGCATGACGAAAACCAGCGTAGACTGCACTATGTATATCTGTGGAGAC
TCCACTGAGTGCTCCAACCTTCTCCTGCAGTACGGTAGCTTCTGTACCCAATTGAACCGC
GCCCTTACAGGCATCGCTGTTGAGCAAGATAAGAATACCCAGGAAGTTTTTGCCCAGGTT
AAGCAGATATACAAAACACCGCCCATTAAGGACTTCGGAGGCTTCAACTTCTCTCAGATA
CTGCCTGACCCCTCCAAGCCATCAAAACGCAGCTTCATTGAGGACCTCTTGTTCAACAAA
GTGACTCTGGCTGATGCTGGCTTCATTAAGCAGTACGGAGATTGCCTGGGAGATATTGCT
GCCAGGGACCTCATCTGCGCCCAGAAGTTTAATGGCCTGACAGTCTTGCCCCCACTTCTG
ACAGACGAGATGATTGCTCAGTACACATCTGCCCTCCTCGCTGGCACCATAACATCCGGA
TGGACATTTGGTGCTGGTGCTGCCCTCCAGATTCCCTTCGCAATGCAGATGGCGTATCGC
TTTAACGGCATCGGTGTCACACAAAACGTGTTGTATGAGAACCAAAAGCTCATCGCTAAC
CAGTTTAATTCTGCTATTGGTAAGATTCAGGACAGCCTGTCATCAACCGCGTCTGCCCTT
GGTAAGTTGCAGGACGTGGTGAACCAGAATGCTCAGGCTTTGAATACTCTGGTGAAGCAA
CTCTCTTCAAATTTCGGCGCTATCTCTTCTGTGTTGAACGACATCCTGAGTCGCCTTGAT
AAGGTGGAAGCTGAAGTTCAAATTGATAGATTGATTACTGGCAGGCTCCAGTCTTTGCAG
ACCTACGTTACACAGCAGCTGATTAGGGCGGCTGAAATTAGAGCTTCCGCCAATCTGGCT
GCAACCAAGATGTCCGAATGCGTCCTGGGTCAGTCAAAGCGCGTTGACTTTTGTGGTAAA
GGCTACCACCTCATGTCATTTCCCCAGTCAGCACCTCACGGAGTAGTGTTCCTCCACGTC
ACCTACGTTCCAGCACAGGAAAAGAATTTTACCACTGCGCCGGCAATCTGTCACGACGGT
AAGGCACACTTCCCCCGCGAGGGCGTATTCGTGTCTAACGGAACTCATTGGTTCGTCACA
CAGAGAAACTTCTATGAGCCTCAGATCATTACCACCGACAATACATTTGTGTCCGGTAAC
TGCGACGTTGTGATTGGAATCGTCAACAACACTGTGTACGATCCACTTCAGCCAGAACTG
GATAGCTTCAAGGAAGAATTGGACAAATATTTCAAAAATCACACTTCACCCGATGTGGAC
CTGGGTGACATTAGTGGTATCAATGCGTCCGTGGTCAATATTCAAAAAGAGATTGACAGG
CTCAACGAAGTGGCCAAGAACCTGAACGAAAGTCTTATCGATCTGCAAGAATTGGGAAAG
TATGAGCAGTACATCAAGTGGCCGTGGTACATTTGGTTGGGTTTTATCGCCGGTCTGATC
GCCATCGTTATGGTTACCATTATGCTTTGCTGCATGACGAGCTGTTGCTCCTGTCTGAAG
GGATGCTGCTCTTGCGGATCATGTTGCAAGTTCGATGAAGACGATAGCGAACCAGTTCTG
AAGGGCGTCAAGCTGCATTACACA
Nucleic acid sequence of the K986P/V987P S2 region sequence
SEQ ID NO: 12
AGTGTGGCTTCTCAAAGCATTATAGCATACACTATGTCTCTTGGTGCCGAAAATTCCGTG
GCCTATTCTAACAATTCAATCGCCATCCCAACCAACTTCACAATTAGCGTGACTACCGAA
ATACTGCCTGTGAGCATGACGAAAACCAGCGTAGACTGCACTATGTATATCTGTGGAGAC
TCCACTGAGTGCTCCAACCTTCTCCTGCAGTACGGTAGCTTCTGTACCCAATTGAACCGC
GCCCTTACAGGCATCGCTGTTGAGCAAGATAAGAATACCCAGGAAGTTTTTGCCCAGGTT
AAGCAGATATACAAAACACCGCCCATTAAGGACTTCGGAGGCTTCAACTTCTCTCAGATA
CTGCCTGACCCCTCCAAGCCATCAAAACGCAGCTTCATTGAGGACCTCTTGTTCAACAAA
GTGACTCTGGCTGATGCTGGCTTCATTAAGCAGTACGGAGATTGCCTGGGAGATATTGCT
GCCAGGGACCTCATCTGCGCCCAGAAGTTTAATGGCCTGACAGTCTTGCCCCCACTTCTG
ACAGACGAGATGATTGCTCAGTACACATCTGCCCTCCTCGCTGGCACCATAACATCCGGA
TGGACATTTGGTGCTGGTGCTGCCCTCCAGATTCCCTTCGCAATGCAGATGGCGTATCGC
TTTAACGGCATCGGTGTCACACAAAACGTGTTGTATGAGAACCAAAAGCTCATCGCTAAC
CAGTTTAATTCTGCTATTGGTAAGATTCAGGACAGCCTGTCATCAACCGCGTCTGCCCTT
GGTAAGTTGCAGGACGTGGTGAACCAGAATGCTCAGGCTTTGAATACTCTGGTGAAGCAA
CTCTCTTCAAATTTCGGCGCTATCTCTTCTGTGTTGAACGACATCCTGAGTCGCCTTGAT
cctccaGAAGCTGAAGTTCAAATTGATAGATTGATTACTGGCAGGCTCCAGTCTTTGCAG
ACCTACGTTACACAGCAGCTGATTAGGGCGGCTGAAATTAGAGCTTCCGCCAATCTGGCT
GCAACCAAGATGTCCGAATGCGTCCTGGGTCAGTCAAAGCGCGTTGACTTTTGTGGTAAA
GGCTACCACCTCATGTCATTTCCCCAGTCAGCACCTCACGGAGTAGTGTTCCTCCACGTC
ACCTACGTTCCAGCACAGGAAAAGAATTTTACCACTGCGCCGGCAATCTGTCACGACGGT
AAGGCACACTTCCCCCGCGAGGGCGTATTCGTGTCTAACGGAACTCATTGGTTCGTCACA
CAGAGAAACTTCTATGAGCCTCAGATCATTACCACCGACAATACATTTGTGTCCGGTAAC
TGCGACGTTGTGATTGGAATCGTCAACAACACTGTGTACGATCCACTTCAGCCAGAACTG
GATAGCTTCAAGGAAGAATTGGACAAATATTTCAAAAATCACACTTCACCCGATGTGGAC
CTGGGTGACATTAGTGGTATCAATGCGTCCGTGGTCAATATTCAAAAAGAGATTGACAGG
CTCAACGAAGTGGCCAAGAACCTGAACGAAAGTCTTATCGATCTGCAAGAATTGGGAAAG
TATGAGCAGTACATCAAGTGGCCGTGGTACATTTGGTTGGGTTTTATCGCCGGTCTGATC
GCCATCGTTATGGTTACCATTATGCTTTGCTGCATGACGAGCTGTTGCTCCTGTCTGAAG
GGATGCTGCTCTTGCGGATCATGTTGCAAGTTCGATGAAGACGATAGCGAACCAGTTCTG
AAGGGCGTCAAGCTGCATTACACA
Nucleic acid sequence of the wildtype 2-1273 sequence of Spike
SEQ ID NO: 13
TTCGTTTTCCTTGTTCTGTTGCCTCTCGTTAGTAGCCAATGCGTCAACCTTACTACTAGA
ACCCAGCTCCCTCCAGCATATACCAACTCTTTCACCAGGGGCGTATATTACCCGGACAAA
GTGTTCCGCTCAAGTGTGCTGCATTCTACGCAGGACCTTTTCTTGCCCTTTTTCAGTAAT
GTTACTTGGTTTCATGCTATCCATGTGTCTGGAACTAACGGAACCAAGCGCTTTGACAAC
CCCGTCCTCCCTTTCAACGATGGCGTGTACTTCGCTTCCACGGAAAAGTCAAACATAATT
CGCGGCTGGATCTTTGGTACAACACTCGACTCAAAGACGCAGAGCCTGCTGATCGTTAAT
AACGCTACAAATGTTGTGATAAAGGTGTGTGAATTTCAGTTCTGCAATGATCCCTTCCTG
GGTGTGTACTACCATAAGAATAACAAGAGCTGGATGGAATCCGAATTTAGGGTTTACAGT
TCCGCTAACAACTGCACATTCGAATACGTAAGCCAGCCATTTCTTATGGATCTTGAGGGC
AAGCAAGGAAACTTCAAGAACTTGAGGGAGTTCGTGTTCAAAAATATCGACGGCTATTTT
AAGATATATAGCAAGCACACTCCAATAAACTTGGTGCGCGACCTGCCCCAGGGATTCTCT
GCTCTGGAGCCCCTGGTGGATCTGCCCATTGGAATAAACATAACTCGCTTTCAAACACTG
CTCGCCCTGCATCGCAGTTACCTCACCCCTGGTGATAGTAGTTCAGGATGGACAGCAGGA
GCCGCCGCATACTACGTCGGCTACCTGCAGCCTAGGACCTTCTTGCTGAAGTACAACGAG
AACGGTACAATAACTGACGCTGTGGACTGCGCTCTGGACCCTCTGTCCGAGACGAAGTGC
ACCCTGAAGAGCTTTACTGTTGAAAAAGGCATTTACCAAACCAGCAACTTCCGCGTCCAG
CCAACCGAGAGCATCGTCAGATTTCCCAACATTACAAATCTGTGTCCCTTCGGCGAGGTG
TTCAACGCCACACGCTTCGCTTCAGTGTACGCATGGAACCGCAAGCGCATATCTAACTGC
GTCGCGGATTATTCTGTCCTCTACAACTCCGCCTCTTTCTCCACCTTCAAGTGCTACGGA
GTGTCACCGACTAAGCTGAACGATCTCTGCTTTACCAACGTCTACGCGGACTCCTTCGTG
ATAAGAGGTGATGAAGTGAGACAAATAGCCCCAGGTCAGACTGGTAAGATCGCAGATTAC
AACTACAAATTGCCTGATGATTTCACTGGTTGCGTTATCGCGTGGAACTCTAATAACCTC
GATTCTAAGGTCGGTGGTAACTACAATTACCTGTACCGCTTGTTTAGGAAGTCAAACCTG
AAGCCTTTCGAGAGGGATATTTCAACCGAAATCTATCAAGCGGGTTCAACACCGTGTAAC
GGTGTGGAAGGATTTAACTGCTACTTCCCCCTGCAGTCTTACGGATTCCAGCCAACCAAT
GGCGTGGGTTACCAACCTTATCGCGTGGTGGTTCTGAGTTTCGAACTGTTGCACGCTCCC
GCCACGGTATGCGGTCCCAAGAAGAGCACTAACTTGGTGAAGAATAAGTGCGTGAATTTC
AATTTCAATGGCCTCACTGGAACTGGAGTGCTGACCGAATCCAATAAGAAGTTCTTGCCC
TTCCAGCAGTTCGGAAGAGACATTGCTGACACAACCGACGCGGTGCGCGATCCTCAGACT
CTGGAGATATTGGACATTACACCATGTTCTTTCGGCGGTGTGTCTGTCATTACTCCGGGC
ACGAATACTAGCAACCAGGTAGCCGTGCTGTACCAAGACGTGAATTGCACAGAGGTTCCC
GTCGCAATTCACGCTGACCAGCTGACCCCCACGTGGAGGGTTTACAGCACTGGTAGTAAC
GTCTTCCAGACGAGAGCCGGTTGCTTGATCGGAGCGGAACATGTGAATAACTCCTACGAG
TGCGACATCCCCATCGGAGCCGGTATATGCGCCTCTTATCAGACACAAACTAACTCACCC
AGGAGAGCCCGCAGTGTGGCTTCTCAAAGCATTATAGCATACACTATGTCTCTTGGTGCC
GAAAATTCCGTGGCCTATTCTAACAATTCAATCGCCATCCCAACCAACTTCACAATTAGC
GTGACTACCGAAATACTGCCTGTGAGCATGACGAAAACCAGCGTAGACTGCACTATGTAT
ATCTGTGGAGACTCCACTGAGTGCTCCAACCTTCTCCTGCAGTACGGTAGCTTCTGTACC
CAATTGAACCGCGCCCTTACAGGCATCGCTGTTGAGCAAGATAAGAATACCCAGGAAGTT
TTTGCCCAGGTTAAGCAGATATACAAAACACCGCCCATTAAGGACTTCGGAGGCTTCAAC
TTCTCTCAGATACTGCCTGACCCCTCCAAGCCATCAAAACGCAGCTTCATTGAGGACCTC
TTGTTCAACAAAGTGACTCTGGCTGATGCTGGCTTCATTAAGCAGTACGGAGATTGCCTG
GGAGATATTGCTGCCAGGGACCTCATCTGCGCCCAGAAGTTTAATGGCCTGACAGTCTTG
CCCCCACTTCTGACAGACGAGATGATTGCTCAGTACACATCTGCCCTCCTCGCTGGCACC
ATAACATCCGGATGGACATTTGGTGCTGGTGCTGCCCTCCAGATTCCCTTCGCAATGCAG
ATGGCGTATCGCTTTAACGGCATCGGTGTCACACAAAACGTGTTGTATGAGAACCAAAAG
CTCATCGCTAACCAGTTTAATTCTGCTATTGGTAAGATTCAGGACAGCCTGTCATCAACC
GCGTCTGCCCTTGGTAAGTTGCAGGACGTGGTGAACCAGAATGCTCAGGCTTTGAATACT
CTGGTGAAGCAACTCTCTTCAAATTTCGGCGCTATCTCTTCTGTGTTGAACGACATCCTG
AGTCGCCTTGATAAGGTGGAAGCTGAAGTTCAAATTGATAGATTGATTACTGGCAGGCTC
CAGTCTTTGCAGACCTACGTTACACAGCAGCTGATTAGGGCGGCTGAAATTAGAGCTTCC
GCCAATCTGGCTGCAACCAAGATGTCCGAATGCGTCCTGGGTCAGTCAAAGCGCGTTGAC
TTTTGTGGTAAAGGCTACCACCTCATGTCATTTCCCCAGTCAGCACCTCACGGAGTAGTG
TTCCTCCACGTCACCTACGTTCCAGCACAGGAAAAGAATTTTACCACTGCGCCGGCAATC
TGTCACGACGGTAAGGCACACTTCCCCCGCGAGGGCGTATTCGTGTCTAACGGAACTCAT
TGGTTCGTCACACAGAGAAACTTCTATGAGCCTCAGATCATTACCACCGACAATACATTT
GTGTCCGGTAACTGCGACGTTGTGATTGGAATCGTCAACAACACTGTGTACGATCCACTT
CAGCCAGAACTGGATAGCTTCAAGGAAGAATTGGACAAATATTTCAAAAATCACACTTCA
CCCGATGTGGACCTGGGTGACATTAGTGGTATCAATGCGTCCGTGGTCAATATTCAAAAA
GAGATTGACAGGCTCAACGAAGTGGCCAAGAACCTGAACGAAAGTCTTATCGATCTGCAA
GAATTGGGAAAGTATGAGCAGTACATCAAGTGGCCGTGGTACATTTGGTTGGGTTTTATC
GCCGGTCTGATCGCCATCGTTATGGTTACCATTATGCTTTGCTGCATGACGAGCTGTTGC
TCCTGTCTGAAGGGATGCTGCTCTTGCGGATCATGTTGCAAGTTCGATGAAGACGATAGC
GAACCAGTTCTGAAGGGCGTCAAGCTGCATTACACA
Nucleic acid sequence of the Δ681-684 sequence of Spike
SEQ ID NO: 14
TTCGTTTTCCTTGTTCTGTTGCCTCTCGTTAGTAGCCAATGCGTCAACCTTACTACTAGA
ACCCAGCTCCCTCCAGCATATACCAACTCTTTCACCAGGGGCGTATATTACCCGGACAAA
GTGTTCCGCTCAAGTGTGCTGCATTCTACGCAGGACCTTTTCTTGCCCTTTTTCAGTAAT
GTTACTTGGTTTCATGCTATCCATGTGTCTGGAACTAACGGAACCAAGCGCTTTGACAAC
CCCGTCCTCCCTTTCAACGATGGCGTGTACTTCGCTTCCACGGAAAAGTCAAACATAATT
CGCGGCTGGATCTTTGGTACAACACTCGACTCAAAGACGCAGAGCCTGCTGATCGTTAAT
AACGCTACAAATGTTGTGATAAAGGTGTGTGAATTTCAGTTCTGCAATGATCCCTTCCTG
GGTGTGTACTACCATAAGAATAACAAGAGCTGGATGGAATCCGAATTTAGGGTTTACAGT
TCCGCTAACAACTGCACATTCGAATACGTAAGCCAGCCATTTCTTATGGATCTTGAGGGC
AAGCAAGGAAACTTCAAGAACTTGAGGGAGTTCGTGTTCAAAAATATCGACGGCTATTTT
AAGATATATAGCAAGCACACTCCAATAAACTTGGTGCGCGACCTGCCCCAGGGATTCTCT
GCTCTGGAGCCCCTGGTGGATCTGCCCATTGGAATAAACATAACTCGCTTTCAAACACTG
CTCGCCCTGCATCGCAGTTACCTCACCCCTGGTGATAGTAGTTCAGGATGGACAGCAGGA
GCCGCCGCATACTACGTCGGCTACCTGCAGCCTAGGACCTTCTTGCTGAAGTACAACGAG
AACGGTACAATAACTGACGCTGTGGACTGCGCTCTGGACCCTCTGTCCGAGACGAAGTGC
ACCCTGAAGAGCTTTACTGTTGAAAAAGGCATTTACCAAACCAGCAACTTCCGCGTCCAG
CCAACCGAGAGCATCGTCAGATTTCCCAACATTACAAATCTGTGTCCCTTCGGCGAGGTG
TTCAACGCCACACGCTTCGCTTCAGTGTACGCATGGAACCGCAAGCGCATATCTAACTGC
GTCGCGGATTATTCTGTCCTCTACAACTCCGCCTCTTTCTCCACCTTCAAGTGCTACGGA
GTGTCACCGACTAAGCTGAACGATCTCTGCTTTACCAACGTCTACGCGGACTCCTTCGTG
ATAAGAGGTGATGAAGTGAGACAAATAGCCCCAGGTCAGACTGGTAAGATCGCAGATTAC
AACTACAAATTGCCTGATGATTTCACTGGTTGCGTTATCGCGTGGAACTCTAATAACCTC
GATTCTAAGGTCGGTGGTAACTACAATTACCTGTACCGCTTGTTTAGGAAGTCAAACCTG
AAGCCTTTCGAGAGGGATATTTCAACCGAAATCTATCAAGCGGGTTCAACACCGTGTAAC
GGTGTGGAAGGATTTAACTGCTACTTCCCCCTGCAGTCTTACGGATTCCAGCCAACCAAT
GGCGTGGGTTACCAACCTTATCGCGTGGTGGTTCTGAGTTTCGAACTGTTGCACGCTCCC
GCCACGGTATGCGGTCCCAAGAAGAGCACTAACTTGGTGAAGAATAAGTGCGTGAATTTC
AATTTCAATGGCCTCACTGGAACTGGAGTGCTGACCGAATCCAATAAGAAGTTCTTGCCC
TTCCAGCAGTTCGGAAGAGACATTGCTGACACAACCGACGCGGTGCGCGATCCTCAGACT
CTGGAGATATTGGACATTACACCATGTTCTTTCGGCGGTGTGTCTGTCATTACTCCGGGC
ACGAATACTAGCAACCAGGTAGCCGTGCTGTACCAAGACGTGAATTGCACAGAGGTTCCC
GTCGCAATTCACGCTGACCAGCTGACCCCCACGTGGAGGGTTTACAGCACTGGTAGTAAC
GTCTTCCAGACGAGAGCCGGTTGCTTGATCGGAGCGGAACATGTGAATAACTCCTACGAG
TGCGACATCCCCATCGGAGCCGGTATATGCGCCTCTTATCAGACACAAACTAACTCACGC
AGTGTGGCTTCTCAAAGCATTATAGCATACACTATGTCTCTTGGTGCCGAAAATTCCGTG
GCCTATTCTAACAATTCAATCGCCATCCCAACCAACTTCACAATTAGCGTGACTACCGAA
ATACTGCCTGTGAGCATGACGAAAACCAGCGTAGACTGCACTATGTATATCTGTGGAGAC
TCCACTGAGTGCTCCAACCTTCTCCTGCAGTACGGTAGCTTCTGTACCCAATTGAACCGC
GCCCTTACAGGCATCGCTGTTGAGCAAGATAAGAATACCCAGGAAGTTTTTGCCCAGGTT
AAGCAGATATACAAAACACCGCCCATTAAGGACTTCGGAGGCTTCAACTTCTCTCAGATA
CTGCCTGACCCCTCCAAGCCATCAAAACGCAGCTTCATTGAGGACCTCTTGTTCAACAAA
GTGACTCTGGCTGATGCTGGCTTCATTAAGCAGTACGGAGATTGCCTGGGAGATATTGCT
GCCAGGGACCTCATCTGCGCCCAGAAGTTTAATGGCCTGACAGTCTTGCCCCCACTTCTG
ACAGACGAGATGATTGCTCAGTACACATCTGCCCTCCTCGCTGGCACCATAACATCCGGA
TGGACATTTGGTGCTGGTGCTGCCCTCCAGATTCCCTTCGCAATGCAGATGGCGTATCGC
TTTAACGGCATCGGTGTCACACAAAACGTGTTGTATGAGAACCAAAAGCTCATCGCTAAC
CAGTTTAATTCTGCTATTGGTAAGATTCAGGACAGCCTGTCATCAACCGCGTCTGCCCTT
GGTAAGTTGCAGGACGTGGTGAACCAGAATGCTCAGGCTTTGAATACTCTGGTGAAGCAA
CTCTCTTCAAATTTCGGCGCTATCTCTTCTGTGTTGAACGACATCCTGAGTCGCCTTGAT
AAGGTGGAAGCTGAAGTTCAAATTGATAGATTGATTACTGGCAGGCTCCAGTCTTTGCAG
ACCTACGTTACACAGCAGCTGATTAGGGCGGCTGAAATTAGAGCTTCCGCCAATCTGGCT
GCAACCAAGATGTCCGAATGCGTCCTGGGTCAGTCAAAGCGCGTTGACTTTTGTGGTAAA
GGCTACCACCTCATGTCATTTCCCCAGTCAGCACCTCACGGAGTAGTGTTCCTCCACGTC
ACCTACGTTCCAGCACAGGAAAAGAATTTTACCACTGCGCCGGCAATCTGTCACGACGGT
AAGGCACACTTCCCCCGCGAGGGCGTATTCGTGTCTAACGGAACTCATTGGTTCGTCACA
CAGAGAAACTTCTATGAGCCTCAGATCATTACCACCGACAATACATTTGTGTCCGGTAAC
TGCGACGTTGTGATTGGAATCGTCAACAACACTGTGTACGATCCACTTCAGCCAGAACTG
GATAGCTTCAAGGAAGAATTGGACAAATATTTCAAAAATCACACTTCACCCGATGTGGAC
CTGGGTGACATTAGTGGTATCAATGCGTCCGTGGTCAATATTCAAAAAGAGATTGACAGG
CTCAACGAAGTGGCCAAGAACCTGAACGAAAGTCTTATCGATCTGCAAGAATTGGGAAAG
TATGAGCAGTACATCAAGTGGCCGTGGTACATTTGGTTGGGTTTTATCGCCGGTCTGATC
GCCATCGTTATGGTTACCATTATGCTTTGCTGCATGACGAGCTGTTGCTCCTGTCTGAAG
GGATGCTGCTCTTGCGGATCATGTTGCAAGTTCGATGAAGACGATAGCGAACCAGTTCTG
AAGGGCGTCAAGCTGCATTACACA
Nucleic acid sequence of the K986P/V987P Δ681-684 sequence
of Spike
SEQ ID NO: 15
TTCGTTTTCCTTGTTCTGTTGCCTCTCGTTAGTAGCCAATGCGTCAACCTTACTACTAGA
ACCCAGCTCCCTCCAGCATATACCAACTCTTTCACCAGGGGCGTATATTACCCGGACAAA
GTGTTCCGCTCAAGTGTGCTGCATTCTACGCAGGACCTTTTCTTGCCCTTTTTCAGTAAT
GTTACTTGGTTTCATGCTATCCATGTGTCTGGAACTAACGGAACCAAGCGCTTTGACAAC
CCCGTCCTCCCTTTCAACGATGGCGTGTACTTCGCTTCCACGGAAAAGTCAAACATAATT
CGCGGCTGGATCTTTGGTACAACACTCGACTCAAAGACGCAGAGCCTGCTGATCGTTAAT
AACGCTACAAATGTTGTGATAAAGGTGTGTGAATTTCAGTTCTGCAATGATCCCTTCCTG
GGTGTGTACTACCATAAGAATAACAAGAGCTGGATGGAATCCGAATTTAGGGTTTACAGT
TCCGCTAACAACTGCACATTCGAATACGTAAGCCAGCCATTTCTTATGGATCTTGAGGGC
AAGCAAGGAAACTTCAAGAACTTGAGGGAGTTCGTGTTCAAAAATATCGACGGCTATTTT
AAGATATATAGCAAGCACACTCCAATAAACTTGGTGCGCGACCTGCCCCAGGGATTCTCT
GCTCTGGAGCCCCTGGTGGATCTGCCCATTGGAATAAACATAACTCGCTTTCAAACACTG
CTCGCCCTGCATCGCAGTTACCTCACCCCTGGTGATAGTAGTTCAGGATGGACAGCAGGA
GCCGCCGCATACTACGTCGGCTACCTGCAGCCTAGGACCTTCTTGCTGAAGTACAACGAG
AACGGTACAATAACTGACGCTGTGGACTGCGCTCTGGACCCTCTGTCCGAGACGAAGTGC
ACCCTGAAGAGCTTTACTGTTGAAAAAGGCATTTACCAAACCAGCAACTTCCGCGTCCAG
CCAACCGAGAGCATCGTCAGATTTCCCAACATTACAAATCTGTGTCCCTTCGGCGAGGTG
TTCAACGCCACACGCTTCGCTTCAGTGTACGCATGGAACCGCAAGCGCATATCTAACTGC
GTCGCGGATTATTCTGTCCTCTACAACTCCGCCTCTTTCTCCACCTTCAAGTGCTACGGA
GTGTCACCGACTAAGCTGAACGATCTCTGCTTTACCAACGTCTACGCGGACTCCTTCGTG
ATAAGAGGTGATGAAGTGAGACAAATAGCCCCAGGTCAGACTGGTAAGATCGCAGATTAC
AACTACAAATTGCCTGATGATTTCACTGGTTGCGTTATCGCGTGGAACTCTAATAACCTC
GATTCTAAGGTCGGTGGTAACTACAATTACCTGTACCGCTTGTTTAGGAAGTCAAACCTG
AAGCCTTTCGAGAGGGATATTTCAACCGAAATCTATCAAGCGGGTTCAACACCGTGTAAC
GGTGTGGAAGGATTTAACTGCTACTTCCCCCTGCAGTCTTACGGATTCCAGCCAACCAAT
GGCGTGGGTTACCAACCTTATCGCGTGGTGGTTCTGAGTTTCGAACTGTTGCACGCTCCC
GCCACGGTATGCGGTCCCAAGAAGAGCACTAACTTGGTGAAGAATAAGTGCGTGAATTTC
AATTTCAATGGCCTCACTGGAACTGGAGTGCTGACCGAATCCAATAAGAAGTTCTTGCCC
TTCCAGCAGTTCGGAAGAGACATTGCTGACACAACCGACGCGGTGCGCGATCCTCAGACT
CTGGAGATATTGGACATTACACCATGTTCTTTCGGCGGTGTGTCTGTCATTACTCCGGGC
ACGAATACTAGCAACCAGGTAGCCGTGCTGTACCAAGACGTGAATTGCACAGAGGTTCCC
GTCGCAATTCACGCTGACCAGCTGACCCCCACGTGGAGGGTTTACAGCACTGGTAGTAAC
GTCTTCCAGACGAGAGCCGGTTGCTTGATCGGAGCGGAACATGTGAATAACTCCTACGAG
TGCGACATCCCCATCGGAGCCGGTATATGCGCCTCTTATCAGACACAAACTAACTCACGC
AGTGTGGCTTCTCAAAGCATTATAGCATACACTATGTCTCTTGGTGCCGAAAATTCCGTG
GCCTATTCTAACAATTCAATCGCCATCCCAACCAACTTCACAATTAGCGTGACTACCGAA
ATACTGCCTGTGAGCATGACGAAAACCAGCGTAGACTGCACTATGTATATCTGTGGAGAC
TCCACTGAGTGCTCCAACCTTCTCCTGCAGTACGGTAGCTTCTGTACCCAATTGAACCGC
GCCCTTACAGGCATCGCTGTTGAGCAAGATAAGAATACCCAGGAAGTTTTTGCCCAGGTT
AAGCAGATATACAAAACACCGCCCATTAAGGACTTCGGAGGCTTCAACTTCTCTCAGATA
CTGCCTGACCCCTCCAAGCCATCAAAACGCAGCTTCATTGAGGACCTCTTGTTCAACAAA
GTGACTCTGGCTGATGCTGGCTTCATTAAGCAGTACGGAGATTGCCTGGGAGATATTGCT
GCCAGGGACCTCATCTGCGCCCAGAAGTTTAATGGCCTGACAGTCTTGCCCCCACTTCTG
ACAGACGAGATGATTGCTCAGTACACATCTGCCCTCCTCGCTGGCACCATAACATCCGGA
TGGACATTTGGTGCTGGTGCTGCCCTCCAGATTCCCTTCGCAATGCAGATGGCGTATCGC
TTTAACGGCATCGGTGTCACACAAAACGTGTTGTATGAGAACCAAAAGCTCATCGCTAAC
CAGTTTAATTCTGCTATTGGTAAGATTCAGGACAGCCTGTCATCAACCGCGTCTGCCCTT
GGTAAGTTGCAGGACGTGGTGAACCAGAATGCTCAGGCTTTGAATACTCTGGTGAAGCAA
CTCTCTTCAAATTTCGGCGCTATCTCTTCTGTGTTGAACGACATCCTGAGTCGCCTTGAT
cctccaGAAGCTGAAGTTCAAATTGATAGATTGATTACTGGCAGGCTCCAGTCTTTGCAG
ACCTACGTTACACAGCAGCTGATTAGGGCGGCTGAAATTAGAGCTTCCGCCAATCTGGCT
GCAACCAAGATGTCCGAATGCGTCCTGGGTCAGTCAAAGCGCGTTGACTTTTGTGGTAAA
GGCTACCACCTCATGTCATTTCCCCAGTCAGCACCTCACGGAGTAGTGTTCCTCCACGTC
ACCTACGTTCCAGCACAGGAAAAGAATTTTACCACTGCGCCGGCAATCTGTCACGACGGT
AAGGCACACTTCCCCCGCGAGGGCGTATTCGTGTCTAACGGAACTCATTGGTTCGTCACA
CAGAGAAACTTCTATGAGCCTCAGATCATTACCACCGACAATACATTTGTGTCCGGTAAC
TGCGACGTTGTGATTGGAATCGTCAACAACACTGTGTACGATCCACTTCAGCCAGAACTG
GATAGCTTCAAGGAAGAATTGGACAAATATTTCAAAAATCACACTTCACCCGATGTGGAC
CTGGGTGACATTAGTGGTATCAATGCGTCCGTGGTCAATATTCAAAAAGAGATTGACAGG
CTCAACGAAGTGGCCAAGAACCTGAACGAAAGTCTTATCGATCTGCAAGAATTGGGAAAG
TATGAGCAGTACATCAAGTGGCCGTGGTACATTTGGTTGGGTTTTATCGCCGGTCTGATC
GCCATCGTTATGGTTACCATTATGCTTTGCTGCATGACGAGCTGTTGCTCCTGTCTGAAG
GGATGCTGCTCTTGCGGATCATGTTGCAAGTTCGATGAAGACGATAGCGAACCAGTTCTG
AAGGGCGTCAAGCTGCATTACACA
tissue plasminogen activator SP
SEQ ID NO: 16
DAMKRGLCCVLLLCGAVFVSPSQEIHARFRR
human IgE immunoglobulin SP
SEQ ID NO: 17
DWTWILFLVAAATRVHS
Amino acid sequence of SARS-CoV-2 Delta variant RBD
SEQ ID NO: 18
RVQPTESIVRFPNITNLCPFGEVFNATRFASVYAWNRKRISNCVADYSVLYNSASFSTFK
CYGVSPTKLNDLCFTNVYADSFVIRGDEVRQIAPGQTGKIADYNYKLPDDFTGCVIAWNS
NNLDSKVGGNYNYRYRLFRKSNLKPFERDISTEIYQAGSKPCNGVEGFNCYFPLQSYGFQ
PTNGVGYQPYRVVVLSFELLHAPATVCGPKKSTNLVKNKCVNF
Amino acid sequence of SARS-CoV-2 Omicron variant RBD
SEQ ID NO: 19
RVQPTESIVRFPNITNLCPFDEVFNATRFASVYAWNRKRISNCVADYSVLYNLAPFFTFK
CYGVSPTKLNDLCFTNVYADSFVIRGDEVRQIAPGQTGNIADYNYKLPDDFTGCVIAWNS
NKLDSKVSGNYNYLYRLFRKSNLKPFERDISTEIYQAGNKPCNGVAGFNCYFPLRSYSFR
PTYGVGHQPYRVVVLSFELLHAPATVCGPKKSTNLVKNKCVNF
Nucleic acid sequence of SARS-CoV-2 Delta variant RBD
SEQ ID NO: 20
CGCGTCCAGCCAACCGAGAGCATCGTCAGATTTCCCAACATTACAAATCTGTGTCCCTTC
GGCGAGGTGTTCAACGCCACACGCTTCGCTTCAGTGTACGCATGGAACCGCAAGCGCATA
TCTAACTGCGTCGCGGATTATTCTGTCCTCTACAACTCCGCCTCTTTCTCCACCTTCAAG
TGCTACGGAGTGTCACCGACTAAGCTGAACGATCTCTGCTTTACCAACGTCTACGCGGAC
TCCTTCGTGATAAGAGGTGATGAAGTGAGACAAATAGCCCCAGGTCAGACTGGTAAGATC
GCAGATTACAACTACAAATTGCCTGATGATTTCACTGGTTGCGTTATCGCGTGGAACTCT
AATAACCTCGATTCTAAGGTCGGTGGTAACTACAATTACaGGTACCGCTTGTTTAGGAAG
TCAAACCTGAAGCCTTTCGAGAGGGATATTTCAACCGAAATCTATCAAGCGGGTTCAAag
CCGTGTAACGGTGTGgAAGGATTTAACTGCTACTTCCCCCTGCAGTCTTACGGATTCCAG
CCAACCAATGGCGTGGGTTACCAACCTTATCGCGTGGTGGTTCTGAGTTTCGAACTGTTG
CACGCTCCCGCCACGGTATGCGGTCCCAAGAAGAGCACTAACTTGGTGAAGAATAAGTGC
GTGAATTTC
Nucleic acid sequence of SARS-CoV-2 Omicron variant RBD
SEQ ID NO: 21
CGCGTCCAGCCAACCGAGAGCATCGTCAGATTTCCCAACATTACAAATCTGTGTCCCTTC
GaCGAGGTGTTCAACGCCACACGCTTCGCTTCAGTGTACGCATGGAACCGCAAGCGCATA
TCTAACTGCGTCGCGGATTATTCTGTCCTCTACAACctCGCCcCTTTCTtCACCTTCAAG
TGCTACGGAGTGTCACCGACTAAGCTGAACGATCTCTGCTTTACCAACGTCTACGCGGAC
TCCTTCGTGATAAGAGGTGATGAAGTGAGACAAATAGCCCCAGGTCAGACTGGTAAcATC
GCAGATTACAACTACAAATTGCCTGATGATTTCACTGGTTGCGTTATCGCGTGGAACTCT
AATAAgCTCGATTCTAAGGTCaGTGGTAACTACAATTACCTGTACCGCTTGTTTAGGAAG
TCAAACCTGAAGCCTTTCGAGAGGGATATTTCAACCGAAATCTATCAAGCGGGTaacAag
CCGTGTAACGGTGTGgcAGGATTTAACTGCTACTTCCCCCTGCgGTCTTACaGcTTCCgG
CCAACCtATGGCGTGGGTcACCAACCTTATCGCGTGGTGGTTCTGAGTTTCGAACTGTTG
CACGCTCCCGCCACGGTATGCGGTCCCAAGAAGAGCACTAACTTGGTGAAGAATAAGTGC
GTGAATTTC
Kozak nucleic acid sequence
SEQ ID NO: 22
GCCACCAUG
polyAC sequence
SEQ ID NO: 23
GAAAAACAAAAAACAAAAAAAACAAAAAAAAAACCAAAAAAACAAAACACA
m6A modification sequence
SEQ ID NO: 24
ACGAGTCCTGGACTGAAACGGACTTGT
3′ exon sequence recognizable by a 3′ catalytic Group I
intron fragment
SEQ ID NO: 25
AAAAUCCGUUGACCUUAAACGGUCGUGUGGGUUCAAGUCCCUCCACCCCCAC
5′ exon sequence recognizable by a 5′ catalytic Group I
intron fragment
SEQ ID NO: 26
GAGACGCUACGGACUU
Exemplary 5′ homology sequence
SEQ ID NO: 27
GGGAGACCCUCGACCGUCGAUUGUCCACUGGUC
Exemplary 3′ homology sequence
SEQ ID NO: 28
ACCAGUGGACAAUCGACGGAUAACAGCAUAUCUAG
T7 Promoter
SEQ ID NO: 29
UAAUACGACUCACUAUAGG
T2A peptide coding sequence
SEQ ID NO: 30
GAGGGCAGAGGAAGUCUUCUAACAUGCGGUGACGUGGAGGAGAAUCCCGGCCCU
P2A peptide coding sequence
SEQ ID NO: 31
GCUACUAACUUCAGCCUGCUGAAGCAGGCUGGAGACGUGGAGGAGAACCCUGGACCU
catalytic Group I intron fragment
SEQ ID NO: 32
AACAAUAGAUGACUUACAACUAAUCGGAAGGUGCAGAGACUCGACGGGAGCUACCCUAAC
GUCAAGACGAGGGUAAAGAGAGAGUCCAAUUCUCAAAGCCAAUAGGCAGUAGCGAAAGCU
GCAAGAGAAUG
5′ catalytic Group I intron fragment
SEQ ID NO: 33
AAAUAAUUGAGCCUUAAAGAAGAAAUUCUUUAAGUGGAUGCUCUCAAACUCAGGGAAACC
UAAAUCUAGUUAUAGACAAGGCAAUCCUGAGCCAAGCCGAAGUAGUAAUUAGUAAG
Nucleic acid sequence of full-length S protein sequence
of SARS-CoV-2
SEQ ID NO: 34
ATGTTCGTTTTCCTTGTTCTGTTGCCTCTCGTTAGTAGCCAATGCGTCAACCTTACTACT
AGAACCCAGCTCCCTCCAGCATATACCAACTCTTTCACCAGGGGCGTATATTACCCGGAC
AAAGTGTTCCGCTCAAGTGTGCTGCATTCTACGCAGGACCTTTTCTTGCCCTTTTTCAGT
AATGTTACTTGGTTTCATGCTATCCATGTGTCTGGAACTAACGGAACCAAGCGCTTTGAC
AACCCCGTCCTCCCTTTCAACGATGGCGTGTACTTCGCTTCCACGGAAAAGTCAAACATA
ATTCGCGGCTGGATCTTTGGTACAACACTCGACTCAAAGACGCAGAGCCTGCTGATCGTT
AATAACGCTACAAATGTTGTGATAAAGGTGTGTGAATTTCAGTTCTGCAATGATCCCTTC
CTGGGTGTGTACTACCATAAGAATAACAAGAGCTGGATGGAATCCGAATTTAGGGTTTAC
AGTTCCGCTAACAACTGCACATTCGAATACGTAAGCCAGCCATTTCTTATGGATCTTGAG
GGCAAGCAAGGAAACTTCAAGAACTTGAGGGAGTTCGTGTTCAAAAATATCGACGGCTAT
TTTAAGATATATAGCAAGCACACTCCAATAAACTTGGTGCGCGACCTGCCCCAGGGATTC
TCTGCTCTGGAGCCCCTGGTGGATCTGCCCATTGGAATAAACATAACTCGCTTTCAAACA
CTGCTCGCCCTGCATCGCAGTTACCTCACCCCTGGTGATAGTAGTTCAGGATGGACAGCA
GGAGCCGCCGCATACTACGTCGGCTACCTGCAGCCTAGGACCTTCTTGCTGAAGTACAAC
GAGAACGGTACAATAACTGACGCTGTGGACTGCGCTCTGGACCCTCTGTCCGAGACGAAG
TGCACCCTGAAGAGCTTTACTGTTGAAAAAGGCATTTACCAAACCAGCAACTTCCGCGTC
CAGCCAACCGAGAGCATCGTCAGATTTCCCAACATTACAAATCTGTGTCCCTTCGGCGAG
GTGTTCAACGCCACACGCTTCGCTTCAGTGTACGCATGGAACCGCAAGCGCATATCTAAC
TGCGTCGCGGATTATTCTGTCCTCTACAACTCCGCCTCTTTCTCCACCTTCAAGTGCTAC
GGAGTGTCACCGACTAAGCTGAACGATCTCTGCTTTACCAACGTCTACGCGGACTCCTTC
GTGATAAGAGGTGATGAAGTGAGACAAATAGCCCCAGGTCAGACTGGTAAGATCGCAGAT
TACAACTACAAATTGCCTGATGATTTCACTGGTTGCGTTATCGCGTGGAACTCTAATAAC
CTCGATTCTAAGGTCGGTGGTAACTACAATTACCTGTACCGCTTGTTTAGGAAGTCAAAC
CTGAAGCCTTTCGAGAGGGATATTTCAACCGAAATCTATCAAGCGGGTTCAACACCGTGT
AACGGTGTGGAAGGATTTAACTGCTACTTCCCCCTGCAGTCTTACGGATTCCAGCCAACC
AATGGCGTGGGTTACCAACCTTATCGCGTGGTGGTTCTGAGTTTCGAACTGTTGCACGCT
CCCGCCACGGTATGCGGTCCCAAGAAGAGCACTAACTTGGTGAAGAATAAGTGCGTGAAT
TTCAATTTCAATGGCCTCACTGGAACTGGAGTGCTGACCGAATCCAATAAGAAGTTCTTG
CCCTTCCAGCAGTTCGGAAGAGACATTGCTGACACAACCGACGCGGTGCGCGATCCTCAG
ACTCTGGAGATATTGGACATTACACCATGTTCTTTCGGCGGTGTGTCTGTCATTACTCCG
GGCACGAATACTAGCAACCAGGTAGCCGTGCTGTACCAAGACGTGAATTGCACAGAGGTT
CCCGTCGCAATTCACGCTGACCAGCTGACCCCCACGTGGAGGGTTTACAGCACTGGTAGT
AACGTCTTCCAGACGAGAGCCGGTTGCTTGATCGGAGCGGAACATGTGAATAACTCCTAC
GAGTGCGACATCCCCATCGGAGCCGGTATATGCGCCTCTTATCAGACACAAACTAACTCA
CCCAGGAGAGCCCGCAGTGTGGCTTCTCAAAGCATTATAGCATACACTATGTCTCTTGGT
GCCGAAAATTCCGTGGCCTATTCTAACAATTCAATCGCCATCCCAACCAACTTCACAATT
AGCGTGACTACCGAAATACTGCCTGTGAGCATGACGAAAACCAGCGTAGACTGCACTATG
TATATCTGTGGAGACTCCACTGAGTGCTCCAACCTTCTCCTGCAGTACGGTAGCTTCTGT
ACCCAATTGAACCGCGCCCTTACAGGCATCGCTGTTGAGCAAGATAAGAATACCCAGGAA
GTTTTTGCCCAGGTTAAGCAGATATACAAAACACCGCCCATTAAGGACTTCGGAGGCTTC
AACTTCTCTCAGATACTGCCTGACCCCTCCAAGCCATCAAAACGCAGCTTCATTGAGGAC
CTCTTGTTCAACAAAGTGACTCTGGCTGATGCTGGCTTCATTAAGCAGTACGGAGATTGC
CTGGGAGATATTGCTGCCAGGGACCTCATCTGCGCCCAGAAGTTTAATGGCCTGACAGTC
TTGCCCCCACTTCTGACAGACGAGATGATTGCTCAGTACACATCTGCCCTCCTCGCTGGC
ACCATAACATCCGGATGGACATTTGGTGCTGGTGCTGCCCTCCAGATTCCCTTCGCAATG
CAGATGGCGTATCGCTTTAACGGCATCGGTGTCACACAAAACGTGTTGTATGAGAACCAA
AAGCTCATCGCTAACCAGTTTAATTCTGCTATTGGTAAGATTCAGGACAGCCTGTCATCA
ACCGCGTCTGCCCTTGGTAAGTTGCAGGACGTGGTGAACCAGAATGCTCAGGCTTTGAAT
ACTCTGGTGAAGCAACTCTCTTCAAATTTCGGCGCTATCTCTTCTGTGTTGAACGACATC
CTGAGTCGCCTTGATAAGGTGGAAGCTGAAGTTCAAATTGATAGATTGATTACTGGCAGG
CTCCAGTCTTTGCAGACCTACGTTACACAGCAGCTGATTAGGGCGGCTGAAATTAGAGCT
TCCGCCAATCTGGCTGCAACCAAGATGTCCGAATGCGTCCTGGGTCAGTCAAAGCGCGTT
GACTTTTGTGGTAAAGGCTACCACCTCATGTCATTTCCCCAGTCAGCACCTCACGGAGTA
GTGTTCCTCCACGTCACCTACGTTCCAGCACAGGAAAAGAATTTTACCACTGCGCCGGCA
ATCTGTCACGACGGTAAGGCACACTTCCCCCGCGAGGGCGTATTCGTGTCTAACGGAACT
CATTGGTTCGTCACACAGAGAAACTTCTATGAGCCTCAGATCATTACCACCGACAATACA
TTTGTGTCCGGTAACTGCGACGTTGTGATTGGAATCGTCAACAACACTGTGTACGATCCA
CTTCAGCCAGAACTGGATAGCTTCAAGGAAGAATTGGACAAATATTTCAAAAATCACACT
TCACCCGATGTGGACCTGGGTGACATTAGTGGTATCAATGCGTCCGTGGTCAATATTCAA
AAAGAGATTGACAGGCTCAACGAAGTGGCCAAGAACCTGAACGAAAGTCTTATCGATCTG
CAAGAATTGGGAAAGTATGAGCAGTACATCAAGTGGCCGTGGTACATTTGGTTGGGTTTT
ATCGCCGGTCTGATCGCCATCGTTATGGTTACCATTATGCTTTGCTGCATGACGAGCTGT
TGCTCCTGTCTGAAGGGATGCTGCTCTTGCGGATCATGTTGCAAGTTCGATGAAGACGAT
AGCGAACCAGTTCTGAAGGGCGTCAAGCTGCATTACACA
Nucleic acid sequence of the RBD amino acid residues 319-542
of S protein
SEQ ID NO: 35
CGCGTCCAGCCAACCGAGAGCATCGTCAGATTTCCCAACATTACAAATCTGTGTCCCTTC
GGCGAGGTGTTCAACGCCACACGCTTCGCTTCAGTGTACGCATGGAACCGCAAGCGCATA
TCTAACTGCGTCGCGGATTATTCTGTCCTCTACAACTCCGCCTCTTTCTCCACCTTCAAG
TGCTACGGAGTGTCACCGACTAAGCTGAACGATCTCTGCTTTACCAACGTCTACGCGGAC
TCCTTCGTGATAAGAGGTGATGAAGTGAGACAAATAGCCCCAGGTCAGACTGGTAAGATC
GCAGATTACAACTACAAATTGCCTGATGATTTCACTGGTTGCGTTATCGCGTGGAACTCT
AATAACCTCGATTCTAAGGTCGGTGGTAACTACAATTACCTGTACCGCTTGTTTAGGAAG
TCAAACCTGAAGCCTTTCGAGAGGGATATTTCAACCGAAATCTATCAAGCGGGTTCAACA
CCGTGTAACGGTGTGGAAGGATTTAACTGCTACTTCCCCCTGCAGTCTTACGGATTCCAG
CCAACCAATGGCGTGGGTTACCAACCTTATCGCGTGGTGGTTCTGAGTTTCGAACTGTTG
CACGCTCCCGCCACGGTATGCGGTCCCAAGAAGAGCACTAACTTGGTGAAGAATAAGTGC
GTGAATTTC
Nucleic acid sequence of the C-terminal Foldon domain of a
T4 fibritin protein
SEQ ID NO: 36
GGAAGCGGCTACATCCCAGAAGCCCCTAGAGACGGACAGGCTTACGTGCGAAAAGACGGC
GAGTGGGTGCTGCTGAGCACATTCCTGGGAAGGAGC
Nucleic acid sequence of the GCN4-based isoleucine zipper
domain
SEQ ID NO: 37
CGAATGAAGCAGATTGAGGATAAAATTGAGGAGATTCTCAGCAAAATTTACCACATA
GAA
AATGAGATCGCTCGGATTAAAAAACTGATCGGAGAAAGA
Nucleic acid sequence of the GS peptide linker
SEQ ID NO: 38
GGCGGAGGAGGCAGCGGCGGAGGAGGCAGC
CVB3 virus IRES
SEQ ID NO: 39
TTAAAACAGCCTGTGGGTTGATCCCACCCACAGGCCCATTGGGCGCTAGCACTCTGGTAT
CACGGTACCTTTGTGCGCCTGTTTTATACCCCCTCCCCCAACTGTAACTTAGAAGTAACA
CACACCGATCAACAGTCAGCGTGGCACACCAGCCACGTTTTGATCAAGCACTTCTGTTAC
CCCGGACTGAGTATCAATAGACTGCTCACGCGGTTGAAGGAGAAAGCGTTCGTTATCCGG
CCAACTACTTCGAAAAACCTAGTAACACCGTGGAAGTTGCAGAGTGTTTCGCTCAGCACT
ACCCCAGTGTAGATCAGGTCGATGAGTCACCGCATTCCCCACGGGCGACCGTGGCGGTGG
CTGCGTTGGCGGCCTGCCCATGGGGAAACCCATGGGACGCTCTAATACAGACATGGTGCG
AAGAGTCTATTGAGCTAGTTGGTAGTCCTCCGGCCCCTGAATGCGGCTAATCCTAACTGC
GGAGCACACACCCTCAAGCCAGAGGGCAGTGTGTCGTAACGGGCAACTCTGCAGCGGAAC
CGACTACTTTGGGTGTCCGTGTTTCATTTTATTCCTATACTGGCTGCTTATGGTGACAAT
TGAGAGATCGTTACCATATAGCTATTGGATTGGCCATCCGGTGACTAATAGAGCTATTAT
ATATCCCTTTGTTGGGTTTATACCACTTAGCTTGAAAGAGGTTAAAACATTACAATTCAT
TGTTAAGTTGAATACAGCAAA
amino acid residues 2-1273 sequence of S protein of
SARS-CoV-2, K986P V987P
SEQ ID NO: 40
FVFLVLLPLVSSQCVNLTTRTQLPPAYTNSFTRGVYYPDKVFRSSVLHSTQDLFLPFFSN
VTWFHAIHVSGTNGTKRFDNPVLPFNDGVYFASTEKSNIIRGWIFGTTLDSKTQSLLIVN
NATNVVIKVCEFQFCNDPFLGVYYHKNNKSWMESEFRVYSSANNCTFEYVSQPFLMDLEG
KQGNFKNLREFVFKNIDGYFKIYSKHTPINLVRDLPQGFSALEPLVDLPIGINITRFQTL
LALHRSYLTPGDSSSGWTAGAAAYYVGYLQPRTFLLKYNENGTITDAVDCALDPLSETKC
TLKSFTVEKGIYQTSNFRVQPTESIVRFPNITNLCPFGEVENATRFASVYAWNRKRISNC
VADYSVLYNSASFSTFKCYGVSPTKLNDLCFTNVYADSFVIRGDEVRQIAPGQTGKIADY
NYKLPDDFTGCVIAWNSNNLDSKVGGNYNYLYRLFRKSNLKPFERDISTEIYQAGSTPCN
GVEGENCYFPLQSYGFQPTNGVGYQPYRVVVLSFELLHAPATVCGPKKSTNLVKNKCVNF
NFNGLTGTGVLTESNKKFLPFQQFGRDIADTTDAVRDPQTLEILDITPCSFGGVSVITPG
TNTSNQVAVLYQDVNCTEVPVAIHADQLTPTWRVYSTGSNVFQTRAGCLIGAEHVNNSYE
CDIPIGAGICASYQTQTNSPRRARSVASQSIIAYTMSLGAENSVAYSNNSIAIPTNFTIS
VTTEILPVSMTKTSVDCTMYICGDSTECSNLLLQYGSFCTQLNRALTGIAVEQDKNTQEV
FAQVKQIYKTPPIKDFGGFNFSQILPDPSKPSKRSFIEDLLFNKVTLADAGFIKQYGDCL
GDIAARDLICAQKFNGLTVLPPLLTDEMIAQYTSALLAGTITSGWTFGAGAALQIPFAMQ
MAYRFNGIGVTQNVLYENQKLIANQFNSAIGKIQDSLSSTASALGKLQDVVNQNAQALNT
LVKQLSSNFGAISSVLNDILSRLDPPEAEVQIDRLITGRLQSLQTYVTQQLIRAAEIRAS
ANLAATKMSECVLGQSKRVDFCGKGYHLMSFPQSAPHGVVFLHVTYVPAQEKNFTTAPAI
CHDGKAHFPREGVFVSNGTHWFVTQRNFYEPQIITTDNTFVSGNCDVVIGIVNNTVYDPL
QPELDSFKEELDKYFKNHTSPDVDLGDISGINASVVNIQKEIDRLNEVAKNLNESLIDLQ
ELGKYEQYIKWPWYIWLGFIAGLIAIVMVTIMLCCMTSCCSCLKGCCSCGSCCKFDEDDS
EPVLKGVKLHYT
amino acid sequence of SARS-CoV-2 strain B.1.351 RBD
SEQ ID NO: 41
RVQPTESIVRFPNITNLCPFGEVFNATRFASVYAWNRKRISNCVADYSVLYNSASFSTFK
CYGVSPTKLNDLCFTNVYADSFVIRGDEVRQIAPGQTGNIADYNYKLPDDFTGCVIAWNS
NNLDSKVGGNYNYLYRLFRKSNLKPFERDISTEIYQAGSTPCNGVKGFNCYFPLQSYGFQ
PTYGVGYQPYRVVVLSFELLHAPATVCGPKKSTNLVKNKCVNF
Nucleic acid sequence encoding SARS-CoV-2 strain B.1.351 RBD
SEQ ID NO: 42
CGCGTCCAGCCAACCGAGAGCATCGTCAGATTTCCCAACATTACAAATCTGTGTCCCTTC
GGCGAGGTGTTCAACGCCACACGCTTCGCTTCAGTGTACGCATGGAACCGCAAGCGCATA
TCTAACTGCGTCGCGGATTATTCTGTCCTCTACAACTCCGCCTCTTTCTCCACCTTCAAG
TGCTACGGAGTGTCACCGACTAAGCTGAACGATCTCTGCTTTACCAACGTCTACGCGGAC
TCCTTCGTGATAAGAGGTGATGAAGTGAGACAAATAGCCCCAGGTCAGACTGGTAACATC
GCAGATTACAACTACAAATTGCCTGATGATTTCACTGGTTGCGTTATCGCGTGGAACTCT
AATAACCTCGATTCTAAGGTCGGTGGTAACTACAATTACCTGTACCGCTTGTTTAGGAAG
TCAAACCTGAAGCCTTTCGAGAGGGATATTTCAACCGAAATCTATCAAGCGGGTTCAACA
CCGTGTAACGGTGTGAAAGGATTTAACTGCTACTTCCCCCTGCAGTCTTACGGATTCCAG
CCAACCTATGGCGTGGGTTACCAACCTTATCGCGTGGTGGTTCTGAGTTTCGAACTGTTG
CACGCTCCCGCCACGGTATGCGGTCCCAAGAAGAGCACTAACTTGGTGAAGAATAAGTGC
GTGAATTTC

1. A circular RNA (circRNA) comprising a nucleic acid sequence encoding an antigenic polypeptide of a SARS-CoV-2 variant, wherein the SARS-CoV-2 variant is a Delta variant or an Omicron variant.

2. The circRNA of claim 1, wherein the antigenic polypeptide comprises a Spike(S) protein or a fragment thereof of the SARS-CoV-2 variant.

3. The circRNA of claim 2, wherein the antigenic polypeptide comprises a receptor-binding domain (RBD) of the S protein.

4. The circRNA of claim 3, wherein the RBD comprises amino acid residues 319 to 542 of a full-length S protein of SARS-CoV-2, wherein the numbering is based on SEQ ID NO: 1.

5. The circRNA of claim 3, wherein the SARS-CoV-2 variant is a Delta variant, and wherein the antigenic polypeptide comprises the amino acid sequence of SEQ ID NO: 18.

6. The circRNA of claim 3, wherein the SARS-CoV-2 variant is an Omicron variant, and wherein the antigenic polypeptide comprises the amino acid sequence of SEQ ID NO: 19.

7. The circRNA of claim 1, wherein the antigenic polypeptide further comprises a multimerization domain.

8. The circRNA of claim 7, wherein the multimerization domain is a C-terminal Foldon (Fd) domain of a T4 fibritin protein that mediates trimerization of the T4 fibritin protein.

9. The circRNA of claim 8, wherein the Fd domain comprises the amino acid sequence of SEQ ID NO: 3.

10. The circRNA of claim 1, further comprising a Kozak sequence operably linked to the nucleic acid sequence encoding the antigenic polypeptide.

11. The circRNA of claim 1, further comprising an in-frame 2A peptide coding sequence operably linked to the 3′ end of the nucleic acid sequence encoding the antigenic polypeptide.

12. The circRNA of claim 1, further comprising an internal ribosomal entry site (IRES) sequence operably linked to the nucleic acid sequence encoding the antigenic polypeptide.

13.-14. (canceled)

15. The circRNA of claim 12, further comprising a polyAC or polyA sequence disposed at the 5′ end of the IRES sequence.

16. The circRNA of claim 1, further comprising an m6A modification motif sequence operably linked to the nucleic acid sequence encoding the antigenic polypeptide.

17. (canceled)

18. The circRNA of claim 1, further comprising a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide.

19. A composition comprising a plurality of circRNAs of claim 1, wherein the antigenic polypeptides corresponding to the plurality of circRNAs are different with respect to each other.

20. (canceled)

21. A circRNA vaccine comprising the circRNA of claim 1.

22.-25. (canceled)

26. A method of treating or preventing a SARS-CoV-2 infection in an individual, comprising administering to the individual an effective amount of the circRNA of claim 1.

27. (canceled)

28. The method of claim 26, wherein the SARS-CoV-2 infection is caused by a Delta or Omicron variant of SARS-CoV-2.

29.-31. (canceled)

32. A linear RNA capable of forming the circRNA of claim 1.

33. A nucleic acid construct comprising a nucleic acid sequence encoding the linear RNA of claim 32, optionally comprising a T7 promoter operably linked to the nucleic acid sequence.
